(19) United States
(12) Patent Application Publication
(10) Pub. No.: US 2025/0281633 A1
(43) Pub. Date: Sep. 11, 2025

(54) HER2 TARGETING FC ANTIGEN BINDING FRAGMENT-DRUG CONJUGATES

(71) Applicant: MERCK PATENT GMBH, DARMSTADT (DE)

(72) Inventors: Sebastian JAEGER, Darmstadt (DE); Christian SCHROETER, Darmstadt (DE)

(73) Assignee: MERCK PATENT GMBH, DARMSTADT (DE)

(21) Appl. No.: 18/558,617

(22) PCT Filed: Apr. 29, 2022
(86) PCT No.: PCT/EP2022/061430

§ 371 Date: Nov. 2, 2023

(30) Foreign Application Priority Data
May 3, 2021 (EP) 21171859.8

(51) Int. Cl. A61K 47/68; A61K 49/00; A61P 35/00; C07K 16/32

(52) U.S. Cl. CPC A61K 47/6855; A61K 47/68031; A61K 49/0058; A61P 35/00; C07K 16/32; A61K 2123/00

(57) ABSTRACT

The invention relates to HER2 targeting Fc antigen binding fragment-drug conjugates (HER2 Fcab-drug conjugates) and the use of the HER2 Fcab-drug conjugates of the present invention for the treatment and/or prevention of hyperproliferative diseases and disorders in mammals, especially humans, and pharmaceutical compositions containing such HER2 Fcab-drug conjugates. Further, the invention relates to HER2 Fcab-label conjugates and diagnostic compositions containing such HER2 Fcab-label conjugates.

The invention relates to HER2 targeting Fc antigen binding fragment-drug conjugates (HER2 Fcab-drug conjugates) and the use of the HER2 Fcab-drug conjugates of the present invention for the treatment and/or prevention of hyperproliferative diseases and disorders in mammals, especially humans, and pharmaceutical compositions containing such HER2 Fcab-drug conjugates. Further, the invention relates to HER2 Fcab-label conjugates and diagnostic compositions containing such HER2 Fcab-label conjugates.

### BACKGROUND OF THE INVENTION

Human Epidermal Growth Factor Receptor 2 (also referred to as HER2, HER2/neu or ErbB-2) is an 85 kDa cytoplasmic transmembrane tyrosine kinase receptor. It is encoded by the c-erbB-2 gene located on the long arm of chromosome 17q and is a member of the HER family (Ross et al., 2003). The HER family normally regulates cell growth and survival, as well as adhesion, migration, differentiation, and other cellular responses (Hudis, C., 2007). Overexpression and amplification of HER2 is observed in the development of a variety of solid cancers including breast (Yarden Y., 2001), gastric (Gravalos et al., 2008), stomach (Ruschoff et al., 2010), colorectal (Ochs et al., 2004), ovarian (Lanitis et al., 2012), pancreatic (Lei et al., 1995), endometrial (Berchuk et al., 1991) and non-small cell lung cancers (Brabender et al., 2001). Breast, colorectal, and gastric cancers accounted for 30% of all diagnosed cancer cases and 24% of all cancer deaths in 2008 (CRUK and WHO World Cancer Report). Breast cancer, in particular, is a leading cause of cancer death among women. HER2 is overexpressed or amplified in 15 to 30% of breast cancers and is associated with poor prognosis, shorter periods of disease-free and overall survival, as well as a more aggressive cancer phenotype (Vinatzer et al., 2005). In breast cancer, about 20% of patients will develop tumors that harbor a genomic alteration involving the amplification of an amplicon on chromosome 17 that contains the HER2 proto-oncogene (Ross J. 2009; and Hicks et al., 2005). Such tumors represent a more aggressive subtype of breast cancer that over-contributes to the mortality of the disease (Hudis C., 2007). A number of HER2 targeting therapies have been approved for treatment of HER2 positive tumors.

Herceptin™ (trastuzumab) is approved for the treatment of metastatic breast cancer in combination with Taxol™ (paclitaxel) and alone for the treatment of HER2 positive breast cancer in patients who have received one or more chemotherapy courses for metastatic disease. As trastuzumab also enhances the efficacy of adjuvant chemotherapy (paclitaxel, docetaxel and vinorelbine) in operable or locally advanced HER2 positive tumors, it is considered standard of care for patients with early or advanced stages of HER2-overexpressing breast cancer. Trastuzumab has also been approved for treatment of HER2 positive metastatic cancer of the stomach or gastroesophageal junction cancer, in combination with chemotherapy (cisplatin and either capecitabine or 5-fluorouracil) in patients who have not received prior treatment for their metastatic disease.

Perjeta™ (pertuzumab) has also been approved for the treatment of HER2 positive metastatic breast cancer in combination with trastuzumab and docetaxel. Pertuzumab targets a different domain of HER2 and has a different mechanism of action than trastuzumab. Specifically, pertuzumab is a HER2 dimerization inhibitor, which prevents HER2 from pairing with other HER receptors (EGFR/HER1, HER3 and HER4).

Kadcyla™ (adotrastuzumab emtansine, T-DM1) is an antibody-drug conjugate, which comprises trastuzumab linked to the cytotoxic agent mertansine (DM1), which disrupts the assembly of microtubules in dividing cells resulting in cell death, and is approved for the treatment of metastatic breast cancer in patients who have received prior treatment with trastuzumab and a taxane chemotherapy.

Tykerb™ (lapatinib) is a small molecule kinase inhibitor that blocks the catalytic action of both HER2 and EGFR. It has been approved in combination with Femara™ (letrozole) for treatment of HER2 positive, hormone receptor positive, metastatic breast cancer in postmenopausal women, and in combination with Xeloda™ (capecitabine) for the treatment of advanced or metastatic HER2-positive breast cancer in patients who have received prior therapy including an anthracycline, a taxane, and Herceptin™. Further drugs are in clinical development.

Although the approved HER2-specific therapies have improved the standard of care of HER2-positive breast and gastric cancers, a significant unmet medical need exists due to intrinsic or acquired resistance to these drugs. Despite trastuzumab's standard of care status for HER2-positive breast cancer, 20-50% of patients from adjuvant settings and around 70% of patients from monotherapy settings go on to develop resistance to trastuzumab (Wolff et al., 2007; and Harris et al, 2007).

There is an emerging trend in cancer therapy towards the selection of patients for treatment based on the assessment of underlying genetic or molecular mechanisms of cancer, as biomarkers. Diagnostic tests based on biomarkers, found to be relevant in particular cancers, have been approved by the FDA to identify patients susceptible to treatment with specific cancer therapies. By May 2013, 15 such diagnostic tests, also known as companion diagnostics, had been approved by the FDA and various others are in development. For example, the Therascreen™ KRAS test is an EGFR immunohistochemistry test which identifies patients having EGFR positive metastatic colorectal cancer with wild-type KRAS genes to be treated with Erbitux™ (cetuximab). The DAKO C-kit PharmDx immunohistochemistry test identifies patients with c-kit positive gastrointestinal stromal tumors susceptible to treatment with Gleevec (imatinib). A number of diagnostic tests have also been approved for identification of HER2 positive tumors for treatment with Herceptin™ (trastuzumab) (Hamburg and Collins, 2010), such as the immunohistochemistry test Herceptest™ and Her2 FISH PharmDx Kit™, which are commonly used together. Further commercially available kits for immunohistochemistry of HER2 positive tumors include Oracle (Leica Biosystems) and Pathway (Ventana). Preclinical and clinical research efforts to identify biomarkers predictive of the clinical response to treatment also have the potential to identify additional patients with “non-traditional” HER2-positive cancers, including colorectal cancer, ovarian cancer and others, which are likely to benefit from HER2 targeting therapies (Gun et al., 2013).

Antibody-drug conjugates (ADCs) advanced rapidly over the last years and were established as a permanent player in the field of oncology providing therapeutic benefit to patients suffering from various cancers. Consequently, five new ADCs were approved by the FDA between 2019 and August 2020 demonstrating the clinical success of this therapeutic class.¹⁻³ ADCs link the great selectivity of monoclonal antibodies with cell killing abilities of highly cytotoxic drugs and expand the therapeutic window by guiding these toxins to tumor cells. To date, approved ADCs and the vast majority of clinical and pre-clinical stage ADCs possess a monoclonal IgG scaffold.⁴ As a result of great success of conventional full-sized ADCs, alternative smaller antibody fragment-based drug conjugates are evolving.^{5,6} Such conjugates consist of Fab-fragments^{7,8}, single chain variable fragments (scFv)^{9,10}, diabodies¹¹ or single-domain antibody-based structures like abdurins¹², nano-¹³⁻¹⁵ or humabodies¹⁶. Their small size allows better solid tumor penetration, due to elevated extravasation from blood vessels into the interstitial tissue space and interstitial diffusion through tissues.^{17,18} However, antibody fragments often do not show better efficacy^{8,16} which may relate to the absence of the Fc domain and its half-life extending function. The interaction of the Fc domain with its natural ligand, the neonatal Fc receptor (FcRn), mediates prolonged circulation of full-length IgG antibodies in the blood stream (e.g. mouse terminal t_{1/2} Trastuzumab vs. FcRn-nonbinding Trastuzumab 212 h vs. 6.9 h¹⁷). Therefore, fragments lacking the Fc portion are often hampered by fast systemic clearance rates and limited exposure (e.g. Trastuzumab Fab, mouse terminal t_{1/2} 4.4 h¹⁷). These findings led to a variety of novel conjugate formats in which small binder fragments were PASylated¹⁹, fused to PEG¹¹, albumin binding domains^{11,13,14,16} or Fc portions²⁰ to improve their in vivo half-life, however, at the cost of increasing the hydrodynamic radius which limits the tumor penetration.

Therefore, ADCs show reduced solid tumor penetration due to their elevated size (150 kDa). This results in inhomogeneous exposition of cancer cells to cytotoxic doses of payload and a lower therapeutic efficacy of ADCs.

In contrast, the known smaller antibody fragment-based drug conjugates (≤50 kDa) show increased solid tumor penetration theoretically resulting in a more homogeneous exposition of cancer cells to the therapeutic. However, their smaller size and the lack of an FcRn binding site causes a shorter half-life of these fragment drug conjugates that counteracts a durable tumor penetration.

Thus, there remains the need to develop novel therapeutic options for the treatment of cancers by ADCs or antibody-fragment based conjugates which show an increased tumor penetration but at the same time a long half-life both mediating an increased therapeutic efficacy.

### SUMMARY OF THE INVENTION

Surprisingly, it has been found, that drug conjugates of another antibody-fragment based format, the Fc antigen binding fragment (Fcab), due to a smaller size and a Fc-mediated half-life extension, in contrast to the known ADCs and the known smaller antibody fragment-based drug conjugates, show at the same time both, an increased tumor penetration and a long half-life both mediating an increased therapeutic efficacy of such Fcab-drug conjugates. Accordingly, an efficient lysosomal delivery was observed for the HER2 Fcab-drug conjugates of the present invention resulting in potent cytotoxic effects in tumor cells. Thus, the HER2 Fcab-drug conjugates of the present invention can be used for the treatment of hyperproliferative diseases and disorders such as cancer.

Fcabs were never described or explored as anti-cancer drug conjugates. Fcabs are IgG1-based homodimeric Fc regions that combine Fc effector functions with an engineered antigen binding site located at the C-terminal structural loops in the C_{H}3 domain.²¹⁻²³. Herein, FcRn binding mediates extraordinary long half-life (terminal t_{1/2} in mice: 60-85 h for Fcabs, 40 h for human Fc^{22,24}) while possessing a molecular size of 50 kDa. Several well characterized Fcabs are directed against the extracellular domains of human epidermal growth factor receptor 2 (HER2). For example, Fcab H10-03-6 was isolated from a yeast surface display (YSD) library of IgG1 Fc regions containing randomized C-terminal structural loop sequences.^{22,25} The reduced thermostability of H10-03-6 was improved by further YSD-based directed evolution protocol resulting in stabilized variants STAB5 and STAB19.²⁶ The most advanced HER2-binding Fcab was isolated from a YSD library and led to the clinically evaluated molecule FS102.²⁴

As shown herein, the favorable pharmacokinetic profile of Fcabs in combination with their small size surprisingly lead to a better and durable penetration of solid tumors by Fcab-based drug conjugates. This results in an elevated overall tumor exposure and better efficacy of the conjugates of the present invention in comparison to other fragment-based drug conjugates of similar size or conventional IgG-based ADCs (concept shown in FIG. 1).

Herein, we present for the first time the generation and functionality of Fcab-drug conjugates. For proof of concept, we selected a diverse set of Fcabs that target the solid tumor antigen HER2. As the intracellular release of the warhead is a prerequisite for an ADC, HER2-dependent uptake rates for selected Fcab molecules were determined on cancer cells. Subsequently, various site-specific conjugation techniques were employed to couple Fcabs with the well-established tubulin inhibitor monomethyl auristatin E (MMAE). Moreover, target-dependent cytotoxicity and stability in serum were evaluated for all Fcab-drug conjugates as well as FcRn and target binding properties compared to parental Fcab molecules. In addition, spheroid assays were applied to assess the impact of target affinity and size on the intracellular accumulation and spheroid penetration. Overall, the disclosed experiments and results emphasize the application of Fcabs for the generation of Fcab-drug-conjugates.

Based on an extensive in vitro characterization, our experiments and results provide the proof-of-concept that the Fcab format is suitable for the generation of stable and cytotoxic drug conjugates. Moreover, we could demonstrate that the 50 kDa Fcab format shows superior spheroid penetration compared to a 150 kDa reference construct. The beneficial spheroid penetration of Fcab-drug conjugates demonstrates a better tumor penetration and an increase in overall tumor exposure and ultimately improved efficacy compared to ADCs.

Thus, the present invention relates to a HER2 Fcab-drug conjugate or a pharmaceutically acceptable salt thereof, comprising the formula Fcab-(L)ₘ-(D)ₙ wherein:
a) Fcab comprises a HER2 Fcab,
b) L comprises a linker,
c) D comprises a drug,
d) m is an integer from 1-5 and n is an integer from 1-10.

In a preferred embodiment of the present invention m is 1 to 3 and n is 1 to 5.

The present invention relates to a HER2 Fcab-drug conjugate according to the present invention wherein the HER2 Fcab is selected from the group consisting of: S5 (native Q295), S5-C265, S5-N^{LLQGA}, S5-N^{G4S-LLQGA}, S5-C^{G4S-LLQGA}, S5-C^{(G4S)2-LLQGA}, S19 (native Q295), S19 (native Q295), FS (native Q295), αH-H10 (Q295), αH-H10^{C265} (D265C), H242-9, STAB1, STAB11, STAB14 and STAB15, having the amino acid sequences as set forth in SEQ ID Nos. 1-16.

A preferred embodiment of the present invention is a HER2 Fcab-drug conjugate according to the present invention wherein the HER2 Fcab is selected from the group consisting of: S5 (native Q295), S5-C265, S5-N^{LLQGA}, S5-N^{G4S-LLQGA}, S5-C^{G4S-LLQGA}, S5-C^{(G4S)2-LLQGA}, S19 (native Q295), S19 (native Q295), FS (native Q295), αH-H10 (Q295) and αH-H10^{C265} (D265C), having the amino acid sequences as set forth in SEQ ID Nos. 1-11.

Another preferred embodiment of the present invention is a HER2 Fcab-drug conjugate according to the present invention wherein the HER2 Fcab is selected from the group consisting of: S5 (native Q295), S5-C265, S5-N^{LLQGA}, S5-N^{G4S-LLQGA}, S5-C^{G4S-LLQGA}, S5-C^{(G4S)2-LLQGA}, S19 (native Q295), S19 (native Q295) and FS (native Q295), having the amino acid sequences as set forth in SEQ ID Nos. 1-9.

Also encompassed by the present invention are HER2 Fcab-drug conjugates according to the present invention wherein the amino acid sequence of the Fcabs is amended or modified by conservative amino acid substitutions. As used herein, the term “conservative substitution” refers to substitutions of amino acids which are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., MOLECULAR BIOLOGY OF THE GENE, The Benjamin/Cummings Pub. Co., p. 224 (4th Edition 1987)).

In general, any drug can be conjugated to the HER2 Fcab-drug conjugate obtained according to the inventive method, as long as it is preferably sufficiently stable to prevent its premature release before reaching the desired target cell, thereby preventing damage to non-target cells and increasing availability at the target site. As the drug is most commonly released in the lysosome following cleavage of the linker molecule, it is important to ensure that the drug remains stable in low pH environments and has the capacity to move into the cytosolic or nuclear compartments of the cell where it takes effect. Similarly, it is desirable that the molecular structure of the drug allows for its conjugation to the linker while avoiding immunogenicity, maintaining the internalization rate of the HER2 Fcab-drug conjugate and promoting or at least not compromising its biological effects, if any (i.e., ADCC, CDCC and CDC). Regardless of the stability of the drug, only a small portion of the administered HER2 Fcab-drug conjugate will typically reach the target cells. Thus, the conjugated drug is preferably potent at low concentrations.

Thus, one embodiment of the present invention is a HER2 Fcab-drug conjugate, wherein the HER2 Fcab is conjugated to a drug selected from a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate). The use of antibody-drug conjugates as ADCs and the HER2 Fcab-drug conjugates of the present invention for the local delivery of cytotoxic or cytostatic agents, i.e. drugs to kill or inhibit tumor cells in the treatment of cancer (Syrigos and Epenetos (1999) Anticancer Research 19:605-614; Niculescu-Duvaz and Springer (1997) Adv. Drg Del. Rev. 26:151-172; U.S. Pat. No. 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet pp. (Mar. 15, 1986): 603-05; Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506). Maximal efficacy with minimal toxicity is sought thereby. Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al. (2000) Jour. of the Nat. Cancer Inst. 92(19): 1573-1581; Mandler et al. (2000) Bioorganic & Med. Chem. Letters 10:1025-1028; Mandler et al (2002) Bioconjugate Chem. 13:786-791), maytansinoids (EP 1391213; Liu et al., (1996) Proc. Natl. Acad. Sci. USA 93:8618-8623),
and calicheamicin (Lode et al. (1998) Cancer Res. 58:2928; Hinman et al. (1993) Cancer Res. 53:3336-3342). The toxins may assert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Suitable drugs envisaged for preparing the HER2 Fcab-drug conjugates of the invention include all cytotoxins commonly utilized in ADCs to date. Most classes of cytotoxins act to inhibit cell division and are classified based on their mechanism of action. Exemplary cytotoxins that are conceivable as part of the inventive HER2 Fcab-drug conjugates include, without limitation, anthracycline, doxorubicin, methotrexate, auristatins including monomethyl auristatin E (MMAE) and monomethyl auristatin F (MMAF), maytansines and their maytansinoids derivatives (DMs), calicheamicins, duocarymycins and pyrrolobenzodiazepine (PBD) dimers.

In one embodiment, the drug moiety is selected from a group consisting of a V-ATPase inhibitor, a pro-apoptotic agent, a Bcl2 inhibitor, an MCL1 inhibitor, a HSP90 inhibitor, an IAP inhibitor, an m Tor inhibitor, a microtubule stabilizer, a microtubule destabilizer, an auristatin, an amanitin, a pyrrolobenzodiazepine, an RNA polymerase inhibitor, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder and a DHFR inhibitor. In some embodiments, the cytotoxic agent is a maytansinoid, wherein the maytansinoid is N(2′)-deacetyl-N(2′)-(3-mercapto-I-oxopropyl)-maytansine (DM1), N (2′)-deacetyl-N(2′)-(4-mercapto-I-oxopentyl)-maytansine (DM3) or N (2′)-deacetyl-N2-(4-mercapto-4-methyl-1-oxopentyl)-maytansine (DM4).

Thus, a preferred embodiment of the present invention is the HER2 Fcab-drug conjugate of the present invention wherein the drug is selected from the group consisting of: anthracycline, doxorubicin, methotrexate, an auristatin including monomethyl auristatin E (MMAE) and monomethyl auristatin F (MMAF), maytansines and their maytansinoids derivatives (DMs), calicheamicins, duocarymycins and pyrrolobenzodiazepine (PBD) dimers, a V-ATPase inhibitor, a pro-apoptotic agent, a Bcl2 inhibitor, an MCL1 inhibitor, a HSP90 inhibitor, an IAP inhibitor, an m Tor inhibitor, a microtubule stabilizer, a microtubule destabilizer, an amanitin, a pyrrolobenzodiazepine, an RNA polymerase inhibitor, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder and a DHFR inhibitor.

In a particular preferred embodiment, the drug is the tubulin inhibitor monomethyl auristatin E (MMAE).

Linkers are preferably designed to be stable in the blood stream (to conform to the increased circulation time of antibodies) and labile at the target site to allow rapid release of the drug. Parameters taken into consideration when designing a suitable linker typically include cleavability of the linker and the position and mechanism of linkage (i.e. conjugation chemistry). Existing linkers are traditionally classified as cleavable or non-cleavable linkers.

Cleavable linkers exploit the change in environment upon internalization of the HER2 Fcab-antigen complex into target cells, resulting in cleavage of the linker and release of the drug into the target cell. Exemplary cleavable linkers that are contemplated for use with the HER2 Fcab drug conjugates provided herein include hydrazone, disulfide and peptide linkers. In contrast to cleavable linkers that rely on distinctive intracellular conditions to release the drug, non-cleavable linkers such as thioether linkers depend solely on the process of proteolytic degradation following HER2 Fcab-antigen internalization and processing in the lysosomal pathway. Linkers for antibody-drug design are well-known in the art and have been reviewed, i.a., by Peters and Brown, Biosci. Rep. 2015 August; 35(4): e00225. One or several drugs can be linked to each HER2 Fcab in order to achieve adequate therapeutic efficacy.

Means and methods for preparing ADCs are described in the art and have been reviewed, i.a., by Peters and Brown (supra). Traditionally, drugs are chemically conjugated to antibodies using conventional techniques, whereby reactive portions of native amino acids are made to interact and bind a specific part of the linker molecule. Examples of reactive groups include the epsilon-amino end of lysine residues and the thiol side chains present in the partially reduced form of cysteine residues. Alternatives to conventional conjugation techniques include conjugation via (i) novel unpaired cysteine residues introduced at specific, controlled sites along the antibody using site-directed mutagenesis, (ii) microbial transglutaminases that recognize glutamine ‘tag’ sequences that can be incorporated into the antibody via plasmids, adding amine-containing drugs to the glutamine side chains, or (iii) non-natural amino acids, such as selenocysteine or acetylphenylalanine introduced into the antibody during transcription, that are available for conjugation with a suitable cytotoxin, for instance in the case of nucleophilic selenocysteine, a positively charged drug molecule.

The drug moiety D can be linked to the HER2 Fcab through linker L. L is any chemical moiety capable of linking the drug moiety to the antibody through covalent bonds. A cross-linking reagent is a bifunctional or multifunctional reagent that can be used to link a drug moiety and an Fcab to form a HER2 Fcab-drug conjugate. HER2 Fcab drug conjugates can be prepared using a cross-linking reagent having a reactive functionality capable of binding to both the drug moiety and the HER2 Fcab. For example, a cysteine, thiol or an amine, e.g. N-terminus or an amino acid side chain, such as lysine of the HER2 Fcab, can form a bond with a functional group of a cross-linking reagent.

In one embodiment, L is a cleavable linker. In another embodiment, L is a non-cleavable linker. In some embodiments, L is an acid-labile linker, photo-labile linker, peptidase cleavable linker, esterase cleavable linker, a disulfide bond cleavable linker, a hydrophilic linker, a procharged linker, or a dicarboxylic acid-based linker.

Suitable cross-linking reagents that form a non-cleavable linker between the drug moiety, for example may tansinoid, and the antibody are well known in the art, and can form non-cleavable linkers that comprise a sulfur atom (such as SMCC) or those that are without a sulfur atom. Preferred cross-linking reagents that form non-cleavable linkers between the drug moiety, for example maytansinoid, and the HER2 Fcab comprises a maleimido- or haloacetyl-based moiety. According to the present invention, such non-cleavable linkers are said to be derived from maleimido- or haloacetyl based moieties.

Cross-linking reagents comprising a maleimido based moiety include but not limited to, N-succinimidyl-4-(maleimidomethyl)cyclohexanecarboxylate (SMCC), sulfo-Succinimidyl 4-(N-maleimidomethyl)cyclohexane-1-carboxylate (sulfo-SMCC), N-succinimidyl-4-(maleimidomethyl)cyclohexane-1-carboxy-(6-amidocaproate), which is a “long chain” analog of SMCC (LC-SMCC), K-maleimidoundeconoic acid N-succinimidylester (KMUA), Y-maleimidobutyric acid N-succinimidylester (GMBS), e-maleimidocaproic acid N-succinimidylester (EMCS), m-maleimidobenzoyl-N-hydroxysuccinimideester (MBS), N—O-maleimidoacetoxy)-succinimide ester (AMSA), succinimidyl-6-(B-maleimidopropionamido) hexanoate (SMPH), N-succinimidyl-4-(p-maleimidophenyl)-butyrate (SMPB), N-(-p-maleomidophenyl)-isocyanate (PMIP) and maleimido-based cross-linking reagents containing a polyethylhene glycol spacer, such as MAL-PEG-NHS. These cross-linking reagents form non-cleavable linkers derived from maleimido-based moieties.

Thus, a preferred embodiment of the present invention is a HER2 Fcab-drug conjugate of the present invention wherein the linker is selected from the linkers described herein.

Another preferred embodiment of the present invention is a HER2 Fcab-drug conjugate of the present invention wherein the linker is selected from the group consisting of an acid-labile linker, a photo-labile linker, a peptidase cleavable linker, an esterase cleavable linker, a disulfide bond cleavable linker, a hydrophilic linker, a procharged linker and a dicarboxylic acid-based linker.

A further preferred embodiment of the present invention is a HER2 Fcab-drug conjugate of the present invention wherein the linker is a disulfide bond cleavable linker.

Each of the embodiments described herein can be combined with any other embodiment described herein not inconsistent with the embodiment with which it is combined. Furthermore, unless incompatible in a given context, wherever a compound is stipulated which is capable of ionization (e.g. protonation or deprotonation), the definition of said compound includes any pharmaceutically acceptable salts thereof. Accordingly, the phrase “or a pharmaceutically acceptable salt thereof” is implicit in the description of all compounds described herein. Embodiments within an aspect as described below can be combined with any other embodiments not inconsistent within the same aspect or a different aspect. For instance, embodiments of any of the treatment methods of the present invention can be combined with any embodiments of the combination products of the present invention or pharmaceutical composition of the present invention, and vice versa. Likewise, any detail or feature given for the treatment methods of the present invention apply—if not inconsistent—to those of the combination products of the present invention and pharmaceutical compositions of the present invention, and vice versa.

The present invention may be understood more readily by reference to the detailed description above and below of the particular and preferred embodiments of the invention and the examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art. So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“A”, “an”, and “the” include plural referents unless the context clearly dictates otherwise. Thus, for example, reference to an antibody refers to one or more antibodies or at least one antibody. As such, the terms “a” (or “an”), “one or more”, and “at least one” are used interchangeably herein.

The term “about” when used to modify a numerically defined parameter refers to any minimal alteration in such parameter that does not change the overall effect, e.g., the efficacy of the agent in treatment of a disease or disorder. In some embodiments, the term “about” means that the parameter may vary by as much as 10% below or above the stated numerical value for that parameter.

“Administering” or “administration of” a drug to a patient (and grammatical equivalents of this phrase) refers to direct administration, which may be administration to a patient by a medical professional or may be self-administration, and/or indirect administration, which may be the act of prescribing a drug, e.g., a physician who instructs a patient to self-administer a drug or provides a patient with a prescription for a drug is administering the drug to the patient.

An “amino acid difference” refers to a substitution, a deletion or an insertion of an amino acid.

“Antibody” is an immunoglobulin (Ig) molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, as well as any protein comprising such antigen-binding fragment or antibody fragment thereof, including fusion proteins (e.g., antibody-drug conjugates, an antibody fused to a cytokine or an antibody fused to a cytokine receptor), antibody compositions with poly-epitopic specificity, and multi-specific antibodies (e.g., bispecific antibodies). The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intra-chain disulfide bridges. Each H chain has, at the N-terminus, a variable domain (V_{H}) followed by three constant domains (C_{H}) for each of the α and γ chains and four C_{H} domains for u and & isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Sties et al. (eds.), Appleton & Lange, Norwalk, CT, 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses based on relatively minor differences in the C_{H} sequence and function, e.g., humans express the following subclasses: IgG1, IgG2A, IgG2B, IgG3, IgG4, IgA1, and IgK1.

“Antigen-binding fragment” of an antibody or “antibody fragment” comprises a portion of an intact antibody, which is still capable of antigen binding. Antigen-binding fragments include, for example, Fab, Fab′, F(ab′)₂, Fd, Fcab and Fv fragments, domain antibodies (dAbs, e.g., shark and camelid antibodies), fragments including CDRs, single chain variable fragment antibodies (scFv), single-chain antibody molecules, multi-specific antibodies formed from antibody fragments, maxibodies, nanobodies, minibodies, intrabodies, diabodies, triabodies, tetrabodies, v-NAR and bis-scFv, linear antibodies (see e.g., U.S. Pat. No. 5,641,870, Example 2; Zapata et al. (1995) Protein Eng. 8HO: 1057), and polypeptides that contain at least a portion of an immunoglobulin that is sufficient to confer specific antigen binding to the polypeptide. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment, which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments were originally produced as pairs of Fab′ fragments which have hinge cysteines between them. Other chemical couplings of antibody fragments are also known.

“Biomarker” generally refers to biological molecules, and quantitative and qualitative measurements of the same, that are indicative of a disease state. “Prognostic biomarkers” correlate with disease outcome, independent of therapy. For example, tumor hypoxia is a negative prognostic marker—the higher the tumor hypoxia, the higher the likelihood that the outcome of the disease will be negative. “Predictive biomarkers” indicate whether a patient is likely to respond positively to a particular therapy, e.g., HER2 profiling is commonly used in breast cancer patients to determine if those patients are likely to respond to Herceptin (trastuzumab, Genentech). “Response biomarkers” provide a measure of the response to a therapy and so provide an indication of whether a therapy is working. For example, decreasing levels of prostate-specific antigen generally indicate that anti-cancer therapy for a prostate cancer patient is working. When a marker is used as a basis for identifying or selecting a patient for a treatment described herein, the marker can be measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of a biomarker in a clinical setting is a clear indication that this parameter was used as a basis for initiating, continuing, adjusting and/or ceasing administration of the treatments described herein.

By “cancer” is meant a collection of cells multiplying in an abnormal manner. As used herein, the term “cancer” refers to all types of cancer, neoplasm, malignant or benign tumors found in mammals, including leukemia, carcinomas, and sarcomas. Exemplary cancers include acute and chronic lymphocytic leukemia, acute granulocytic leukemia, adrenal cortex cancer, bladder cancer, brain cancer, breast cancer, cervical cancer, cervical hyperplasia, chorion cancer, chronic granulocytic leukemia, chronic lymphocytic leukemia, colon cancer, endometrial cancer, kidney cancer, biliary tract cancer, hepatoma, liver cancer, esophageal cancer, essential thrombocytosis, genitourinary carcinoma, glioma, glioblastoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, chondrosarcoma, multiple myeloma, mycosis fungoides, myeloid and lymphocytic leukemia, neuroblastoma, non-Hodgkin's lymphoma, non-small cell lung cancer, osteogenic sarcoma, ovarian carcinoma, pancreatic carcinoma, polycythemia vera, primary brain carcinoma, primary macroglobulinemia, prostatic cancer, renal cell cancer, rhabdomyosarcoma, skin cancer, small-cell lung cancer, soft-tissue sarcoma, squamous cell cancer, stomach cancer, testicular cancer, thyroid cancer and Wilms' tumor.

“CDRs” are the complementarity determining region amino acid sequences of an antibody, antibody fragment or antigen-binding fragment. These are the hypervariable regions of immunoglobulin heavy and light chains. There are three heavy chain and three light chain CDRs (or CDR regions) in the variable portion of an immunoglobulin.

“Clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity, or side effect.

“Combination” as used herein refers to the provision of a first active modality in addition to one or more further active modalities (wherein one or more active modalities may be fused). Contemplated within the scope of the combinations described herein, are any regimen of combination modalities or partners (i.e., active compounds, components or agents), encompassed in single or multiple compounds and compositions. It is understood that any modalities within a single composition, formulation or unit dosage form (i.e., a fixed-dose combination) must have the identical dose regimen and route of delivery. It is not intended to imply that the modalities must be formulated for delivery together (e.g., in the same composition, formulation or unit dosage form). The combined modalities can be manufactured and/or formulated by the same or different manufacturers. The combination partners may thus be, e.g., entirely separate pharmaceutical dosage forms or pharmaceutical compositions that are also sold independently of each other.

“Combination therapy”, “in combination with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, four or more modalities may be used in a combination therapy. Additionally, the combination therapies provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject.

“Complete response” or “complete remission” refers to the disappearance of all signs of cancer in response to treatment. This does not always mean the cancer has been cured.

“Comprising”, as used herein, is intended to mean that the compositions and methods include the recited elements, but not excluding others. “Consisting essentially of”, when used to define compositions and methods, shall mean excluding other elements of any essential significance to the composition or method.

“Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“Drug conjugate” or “drug” according to the present invention is a conjugate of a HER2 Fcab according to the present invention and a drug selected from the group including but not limited to anthracycline, doxorubicin, methotrexate, an auristatin including monomethyl auristatin E (MMAE) and monomethyl auristatin F (MMAF), maytansines and their maytansinoids derivatives (DMs), calicheamicins, duocarymycins and pyrrolobenzodiazepine (PBD) dimers, a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an amanitin, a pyrrolobenzodiazepine, an RNA polymerase inhibitor, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder or a DHFR inhibitor.

“Fcab” according to the present invention is an IgG1-based homodimeric Fc region that combine Fc effector functions with an engineered antigen binding site located at the C-terminal structural loops in the C_{H}3 domain.²¹⁻²³. Antigen-binding Fc fragments (also referred to as Fcab™ [Fc fragment with antigen binding]) comprising e.g., a modified IgG1 Fc domain which binds to HER2 with high affinity, are described in WO 2009/132876 A 1 and WO 2009/000006 A 1 which are hereby incorporated by reference in their entirety. Specific binding members described herein include antigen binding Fc fragments described herein which each has one or more amino acid modifications in at least one structural loop region, wherein the modified structural loop region specifically binds to an epitope of an antigen, e.g. HER2, to which an unmodified Fc fragment does not significantly bind.

“Fc” is a fragment comprising the carboxy-terminal portions of both H chains held together by disulfides. The effector functions of antibodies are determined by sequences in the Fc region, the region which is also recognized by Fc receptors (FcR) found on certain types of cells. Antigen-binding Fc fragments may comprise an antigen-binding site engineered into one or more structural loop regions of a constant domain of the Fc fragment, e.g. the CH2 or CH3 domain. The preparation of antigen-binding Fc fragments is described in WO 2006/072620 and WO2009/132876. A specific binding member for use in the present invention preferably is, or comprises, an antigen binding Fc fragment, also referred to as Fcab™. More preferably, a specific binding member for use in the present invention is an antigen-binding Fc fragment. The specific binding member may be an IgA1, IgA2, IgD, IgE, IgG, IgG2, IgG3, IgG4 or IgM antigen-binding Fc fragment. Most preferably, a specific binding member as referred to herein is an IgG1 (e.g., human IgG1) antigen-binding Fc fragment. In certain embodiments, a specific binding member is an IgG1 antigen-binding Fc fragment comprising a hinge or portion thereof, a CH2 domain and a CH3 domain.

“Fv” is the minimum antibody fragment, which contains a complete antigen-recognition and antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen-binding specificity to the antibody. However, even a single variable domain (or half of an Fv comprising only three HVRs specific for an antigen) has the ability to recognize and bind antigen, although at a lower affinity than the entire binding site.

“Human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (see e.g., Hoogenboom and Winter (1991), JMB 227:381; Marks et al. (1991) JMB 222:581). Also available for the preparation of human monoclonal antibodies are methods described in Cole et al. (1985) Monoclonal Antibodies and Cancer Therapy, Alan R. Liss, page 77; Boerner et al. (1991), J. Immunol. 147 (I): 86; van Dijk and van de Winkel (2001) Curr. Opin. Pharmacol. 5:368). Human antibodies can be prepared by administering the antigen to a transgenic animal that has been modified to produce such antibodies in response to antigenic challenge but whose endogenous loci have been disabled, e.g., immunized xenomice (see e.g., U.S. Pat. Nos. 6,075,181; and 6,150,584 regarding XENOMOUSE technology). See also, for example, Li et al. (2006) PNAS USA, 103:3557, regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit, or non-human primate having the desired specificity, affinity and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and no more than 3 in the L chain. The humanized antibody optionally will also comprise at least a portion of an immunoglobulin constant region (Fc), typically that of a human immunoglobulin. For further details, see e.g., Jones et al. (1986) Nature 321:522; Riechmann et al. (1988), Nature 332:323; Presta (1992) Curr. Op. Struct. Biol. 2:593; Vaswani and Hamilton (1998), Ann. Allergy, Asthma & Immunol. 1:105; Harris (1995) Biochem. Soc. Transactions 23:1035; Hurle and Gross (1994) Curr. Op. Biotech. 5:428; and U.S. Pat. Nos. 6,982,321 and 7,087,409.

“Infusion” or “infusing” refers to the introduction of a drug-containing solution into the body through a vein for therapeutic purposes. Generally, this is achieved via an intravenous (IV) bag.

“Metastatic” cancer refers to cancer which has spread from one part of the body (e.g., the lung) to another part of the body.

“Monoclonal antibody”, as used herein, refers to an antibody obtained from a population of substantially homogeneous antibodies, i.e., the individual antibodies comprising the population are identical except for possible naturally occurring mutations and/or post-translation modifications (e.g., isomerizations and amidations) that may be present in minor amounts. Monoclonal antibodies are highly specific, being directed against a single antigenic site. In contrast to polyclonal antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture and uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including, for example, the hybridoma method (e.g., Kohler and Milstein (1975) Nature 256:495; Hongo et al. (1995) Hybridoma 14 (3): 253; Harlow et al. (1988) Antibodies: A Laboratory Manual (Cold Spring Harbor Laboratory Press, 2^{nd} ed.; Hammerling et al. (1981) In: Monoclonal Antibodies and T-Cell Hybridomas 563 (Elsevier, N.Y.), recombinant DNA methods (see e.g., U.S. Pat. No. 4,816,567), phage-display technologies (see e.g., Clackson et al. (1991) Nature 352:624; Marks et al. (1992) JMB 222:581; Sidhu et al. (2004) JMB 338(2): 299; Lee et al. (2004) JMB 340(5): 1073; Fellouse (2004) PNAS USA 101(34): 12467; and Lee et al. (2004) J. Immunol. Methods 284(1-2): 119), and technologies for producing human or human-like antibodies in animals that have parts or all of the human immunoglobulin loci or genes encoding human immunoglobulin sequences (see e.g., WO 1998/24893; WO 1996/34096; WO 1996/33735; WO 1991/10741; Jakobovits et al. (1993) PNAS USA 90:2551; Jakobovits et al. (1993) Nature 362:255; Bruggemann et al. (1993) Year in Immunol. 7:33; U.S. Pat. Nos. 5,545,807; 5,545,806; 5,569,825; 5,625,126; 5,633,425; and U.S. Pat. No. 5,661,016; Marks et al. (1992) Bio/Technology 10:779; Lonberg et al. (1994) Nature 368:856; Morrison (1994) Nature 368:812; Fishwild et al. (1996) Nature Biotechnol. 14:845; Neuberger (1996), Nature Biotechnol. 14:826; and Lonberg and Huszar (1995), Intern. Rev. Immunol. 13:65-93).

The monoclonal antibodies herein specifically include chimeric antibodies (immunoglobulins) in which a portion of the heavy and/or light chain is identical to or homologous to corresponding sequences in antibodies derived from a particular species or belonging to a particular antibody class or subclass, while the remainder of the chain(s) is (are) identical to or homologous to corresponding sequences in antibodies derived from another species or belonging to another antibody class or subclass, as well as fragments of such antibodies, so long as they exhibit the desired biological activity (see e.g., U.S. Pat. No. 4,816,567; Morrison et al. (1984) PNAS USA, 81:6851).

“Objective response” refers to a measurable response, including complete response (CR) or partial response (PR).

“Partial response” refers to a decrease in the size of one or more tumors or lesions, or in the extent of cancer in the body, in response to treatment.

“Patient” and “subject” are used interchangeably herein to refer to a mammal in need of treatment for a cancer. Generally, the patient is a human diagnosed or at risk for suffering from one or more symptoms of a cancer. In certain embodiments a “patient” or “subject” may refer to a non-human mammal, such as a non-human primate, a dog, cat, rabbit, pig, mouse, or rat, or animals used, e.g., in screening, characterizing, and evaluating drugs and therapies.

“Percent (%) sequence identity” with respect to a peptide or polypeptide sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2 or ALIGN software. Those skilled in the art can determine appropriate parameters for measuring alignment, including any algorithms needed to achieve maximal alignment over the full length of the sequences being compared.

“Pharmaceutically acceptable” indicates that the substance or composition must be compatible chemically and/or toxicologically, with the other ingredients comprising a formulation, and/or the mammal being treated therewith. “Pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof.

“Pharmaceutically acceptable salt” forms of HER2 Fcab-drug conjugate are for the most part prepared by conventional methods. If the HER2 Fcab-drug conjugate of the present invention contains a carboxyl group, one of its suitable salts can be formed by reacting the compound of the present invention with a suitable base to give the corresponding base-addition salt. Such bases are, for example, alkali metal hydroxides, including potassium hydroxide, sodium hydroxide and lithium hydroxide; alkaline-earth metal hydroxides, such as barium hydroxide and calcium hydroxide; alkali metal alkoxides, for example potassium ethoxide and sodium propoxide; and various organic bases, such as piperidine, diethanolamine and N-methylglutamine.

Furthermore, the base salts of the HER2 Fcab-drug conjugate of the present invention include aluminium, ammonium, calcium, copper, iron (III), iron (II), lithium, magnesium, manganese (III), manganese (II), potassium, sodium and zinc salts, but this is not intended to represent a restriction.

Of the above-mentioned salts, preference is given to ammonium; the alkali metal salts sodium and potassium, and the alkaline-earth metal salts calcium and magnesium. Salts of the HER2 Fcab-drug conjugate of the present invention which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzyl-ethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)methylamine (tromethamine), but this is not intended to represent a restriction.

As mentioned, the pharmaceutically acceptable base-addition salts of HER2 Fcab-drug conjugate are formed with metals or amines, such as alkali metals and alkaline-earth metals or organic amines. Preferred metals are sodium, potassium, magnesium and calcium. Preferred organic amines are N,N′-dibenzylethylene-diamine, chloroprocaine, choline, diethanolamine, ethylenediamine, N-methyl-D-glucamine and procaine.

The base-addition salts of the HER2 Fcab-drug conjugate of the present invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

“Prodrug” refers to derivatives of the HER2 Fcab-drug conjugates of the present invention which have been modified by means of, for example, alkyl or acyl groups (see also amino- and hydroxyl-protecting groups below), sugars or oligopeptides and which are rapidly cleaved or liberated in the organism to form the effective molecules. These also include biodegradable polymer derivatives of the HER2 Fcab-drug conjugate of the present invention, as described, for example, in Int. J. Pharm. 115 (1995), 61-67.

“Recurrent” cancer is one which has regrown, either at the initial site or at a distant site, after a response to initial therapy, such as surgery. A locally “recurrent” cancer is cancer that returns after treatment in the same place as a previously treated cancer.

“Reduction” of a symptom or symptoms (and grammatical equivalents of this phrase) refers to decreasing the severity or frequency of the symptom(s), or elimination of the symptom(s).

“Single-chain Fv”, also abbreviated as “sFv” or “scFv”, are antibody fragments that comprise the V_{H} and V_{L} antibody domains connected into a single polypeptide chain. In some embodiments, the sFv polypeptide further comprises a polypeptide linker between the V_{H} and V_{L} domains which enables the sFv to form the desired structure for antigen binding. For a review of the sFv, see e.g., Pluckthun (1994), In: The Pharmacology of Monoclonal Antibodies, vol. 113, Rosenburg and Moore (eds.), Springer-Verlag, New York, pp. 269.

“Solvates” refer to adductions of inert solvent molecules onto the HER2 Fcab-drug conjugates of the invention which form owing to their mutual attractive force. Solvates are, for example, hydrates, such as monohydrates or dihydrates, or alcoholates, i.e. addition compounds with alcohols, such as, for example, with methanol or ethanol.

By “substantially identical” is meant (1) a query amino acid sequence exhibiting at least 75%, 85%, 90%, 95%, 99% or 100% amino acid sequence identity to a subject amino acid sequence or (2) a query amino acid sequence that differs in not more than 20%, 30%, 20%, 10%, 5%, 1% or 0% of its amino acid positions from the amino acid sequence of a subject amino acid sequence and wherein a difference in an amino acid position is any of a substitution, deletion or insertion of an amino acid.

“Systemic” treatment is a treatment, in which the drug substance travels through the bloodstream, reaching and affecting cells all over the body.

“Therapeutically effective amount” of HER2 Fcab-drug conjugate, refers to an amount effective, at dosages and for periods of time necessary, that, when administered to a patient with a cancer, will have the intended therapeutic effect, e.g., alleviation, amelioration, palliation, or elimination of one or more manifestations of the cancer in the patient, or any other clinical result in the course of treating a cancer patient. A therapeutic effect does not necessarily occur by administration of one dose and may occur only after administration of a series of doses. Thus, a therapeutically effective amount may be administered in one or more administrations. Such therapeutically effective amount may vary according to factors such as the disease state, age, sex, and weight of the individual, and the ability of a HER2 Fcab-drug conjugate to elicit a desired response in the individual. A therapeutically effective amount is also one in which any toxic or detrimental effects of a HER2 Fcab-drug conjugate are outweighed by the therapeutically beneficial effects. The term “effective amount” denotes the amount of a medicament or of a pharmaceutical active compound which causes in a tissue, system, animal or human a biological or medical response which is sought or desired, for example, by a researcher or physician.

In addition, the term “therapeutically effective amount” denotes an amount which, compared with a corresponding subject who has not received this amount, has the following consequence: improved treatment, healing, prevention or elimination of a disease, syndrome, disease state, complaint, disorder or prevention of side effects or also a reduction in the progress of a disease, complaint or disorder. The term “therapeutically effective amount” also encompasses the amounts which are effective for increasing normal physiological function.

“Treating” or “treatment of” a condition or patient refers to taking steps to obtain beneficial or desired results, including clinical results. For purposes of this invention, beneficial or desired clinical results include, but are not limited to, alleviation, amelioration of one or more symptoms of a cancer; diminishment of extent of disease; delay or slowing of disease progression; amelioration, palliation, or stabilization of the disease state; or other beneficial results. It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

“Variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

As used herein, a plurality of items, structural elements, compositional elements, and/or materials may be presented in a common list for convenience. However, these lists should be construed as though each member of the list is individually identified as a separate and unique member.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

When discovering and developing therapeutic agents, the person skilled in the art attempts to optimise pharmacokinetic parameters while retaining desirable in-vitro properties. It is reasonable to assume that many compounds with poor pharmacokinetic profiles are susceptible to oxidative metabolism. In-vitro liver microsomal assays currently available provide valuable information on the course of oxidative metabolism of this type, which in turn permits the rational design of deuterated compounds of the present invention with improved stability through resistance to such oxidative metabolism. Significant improvements in the pharmacokinetic profiles of the HER2 Fcab-drug conjugates of the present invention are thereby obtained and can be expressed quantitatively in terms of increases in the in-vivo half-life (T/2), concentration at maximum therapeutic effect (Cₘₐₓ), area under the dose response curve (AUC), and F; and in terms of reduced clearance, dose and costs of materials.

The invention also relates, in particular, to a medicament comprising at least one HER2 Fcab-drug conjugate according to the invention for use in the treatment and/or prophylaxis of physiological and/or pathophysiological states.

Physiological and/or pathophysiological states are taken to mean physiological and/or pathophysiological states which are medically relevant, such as, for example, diseases or illnesses and medical disorders, complaints, symptoms or complications and the like, in particular diseases.

A preferred embodiment of the present invention is a medicament comprising at least one HER2 Fcab-drug conjugate according to the present invention for use in the treatment and/or prophylaxis of physiological and/or pathophysiological states, selected from the group consisting of hyperproliferative diseases and disorders.

A yet more preferred embodiment of the present invention is a medicament according to the present invention for use in the treatment and/or prophylaxis of physiological and/or pathophysiological states, selected from the group consisting of hyperproliferative diseases and disorders, wherein the hyperproliferative disease or disorder is cancer.

Another preferred embodiment of the present invention is a medicament according to the present invention for use in the treatment of cancer, wherein the cancer is selected from the group consisting of acute and chronic lymphocytic leukemia, acute granulocytic leukemia, adrenal cortex cancer, bladder cancer, brain cancer, breast cancer, cervical cancer, cervical hyperplasia, chorion cancer, chronic granulocytic leukemia, chronic lymphocytic leukemia, colon cancer, endometrial cancer, kidney cancer, biliary tract cancer, hepatoma, liver cancer, esophageal cancer, essential thrombocytosis, genitourinary carcinoma, glioma, glioblastoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, chondrosarcoma, multiple myeloma, mycosis fungoides, myeloid and lymphocytic leukemia, neuroblastoma, non-Hodgkin's lymphoma, non-small cell lung cancer, osteogenic sarcoma, ovarian carcinoma, pancreatic carcinoma, polycythemia vera, primary brain carcinoma, primary macroglobulinemia, prostatic cancer, renal cell cancer, rhabdomyosarcoma, skin cancer, small-cell lung cancer, soft-tissue sarcoma, squamous cell cancer, stomach cancer, testicular cancer, thyroid cancer and Wilms' tumor.

Particular preference is given, in particular, to physiological and/or pathophysiological states which are connected to HER2. Thus, the present invention relates to a medicament according to the present invention for use in the treatment of HER2-positive cancers.

A cancer as referred to herein may be a gastric cancer, breast cancer, colorectal cancer, ovarian cancer, pancreatic cancer, lung cancer (for example, non-small cell lung cancer), stomach cancer, or endometrial cancer. All of these cancers have been shown to overexpress HER2. Preferably, the cancer is gastric cancer, breast cancer, or colorectal cancer. More preferably, the cancer is gastric cancer or breast cancer. In one preferred embodiment, the cancer is gastric cancer. Gastric cancer, as referred to herein, includes esophageal cancer. In another preferred embodiment, the cancer is breast cancer. The HER2 gene copy number of the cancer is as set out above. Such a cancer may be referred to as HER2-positive (HER2+) or as overexpressing HER2. Thus, a cancer, as referred to herein, may be HER2-positive. In addition, or alternatively, a cancer as referred to herein may overexpress HER2. Whether a cancer is HER2-positive or overexpresses HER2 may, for example, be determined initially using immunohistochemistry (IHC), optionally followed by methods such as qPCR as outlined above.

A further preferred embodiment is a medicament according to the present invention for use in the treatment solid cancers including breast cancer, gastric cancer, stomach cancer, colorectal cancer, ovarian cancer, pancreatic cancer, endometrial cancer or non-small cell lung cancer.

It is intended that the medicaments disclosed above include a corresponding use of the HER2 Fcab-drug conjugate according to the invention for the preparation of a medicament for the treatment and/or prophylaxis of the above physiological and/or pathophysiological states.

It is additionally intended that the medicaments disclosed above include a corresponding method for the treatment and/or prophylaxis of the above physiological and/or pathophysiological states in which at least one HER2 Fcab-drug conjugate according to the invention is administered to a patient in need of such a treatment.

Accordingly, also an embodiment of the present invention is the use of a HER2 Fcab-drug conjugate according to the present invention for the treatment of cancer.

Accordingly, also an embodiment of the present invention is the use of a HER2 Fcab-drug conjugate for the manufacture of a medicament for the treatment of cancer.

Accordingly, also an embodiment of the present invention is a method for treating cancer in a subject wherein the method comprises administering the HER2 Fcab-drug conjugate or the pharmaceutical preparation according to the present invention to the subject.

Accordingly, also an embodiment of the present invention is the use of a method for the treatment of cancer comprising administering the HER2 Fcab-drug conjugate or the pharmaceutical preparation according to the present invention to a subject in need thereof.

In one embodiment, the HER2 Fcab-drug conjugate of the invention is used in the treatment of a human subject. The main expected benefit in the treatment with the therapeutic combination of the HER2 Fcab and the drug is a gain in risk/benefit ratio for these human patients. The administration of the HER2 Fcab-drug conjugates of the invention may be advantageous over the individual therapeutic agents in that the combinations of the HER2 Fcab and the drug may provide one or more of the following improved properties when compared to the individual administration of a single therapeutic agent alone: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, and/or vi) an increase in the bioavailability of one or both of the therapeutic agents.

In certain embodiments, the invention provides for the treatment of diseases, disorders, and conditions characterized by excessive or abnormal cell proliferation. Such diseases include a proliferative or hyperproliferative disease. Examples of proliferative and hyperproliferative diseases include cancer and myeloproliferative disorders.

In another embodiment, the cancer is selected from carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma, cervical cancer, brain cancer, stomach cancer, bladder cancer, hepatoma, breast cancer, colon carcinoma, biliary tract cancer, and head and neck cancer. The disease or medical disorder in question may be selected from any of those disclosed in WO2015118175, WO2018029367, WO2018208720, PCT/US18/12604, PCT/US19/47734, PCT/US19/40129, PCT/US19/36725, PCT/US19/732271, PCT/US19/38600, PCT/EP2019/061558.

In one embodiment, the cancer is selected from: appendiceal cancer, bladder cancer, breast cancer, cervical cancer, colorectal cancer, endometrial cancer, esophageal cancer (in particular esophageal squamous cell carcinoma), fallopian tube cancer, gastric cancer, glioma (such as diffuse intrinsic pontine glioma), head and neck cancer (in particular head and neck squamous cell carcinoma and oropharyngeal cancer), leukemia (in particular acute lymphoblastic leukemia, acute myeloid leukemia) lung cancer (in particular non-small cell lung cancer), lymphoma (in particular Hodgkin's lymphoma, non-Hodgkin's lymphoma), melanoma, mesothelioma (in particular malignant pleural mesothelioma), Merkel cell carcinoma, neuroblastoma, oral cancer, osteosarcoma, ovarian cancer, prostate cancer, renal cancer, salivary gland tumor, sarcoma (in particular Ewing's sarcoma or rhabdomyosarcoma) squamous cell carcinoma, soft tissue sarcoma, thymoma, thyroid cancer, urothelial cancer, uterine cancer, vaginal cancer, vulvar cancer or Wilms tumor. In a further embodiment, the cancer is selected from: appendiceal cancer, bladder cancer, cervical cancer, colorectal cancer, esophageal cancer, head and neck cancer, melanoma, mesothelioma, non-small-cell lung cancer, prostate cancer and urothelial cancer. In a further embodiment, the cancer is selected from cervical cancer, endometrial cancer, head and neck cancer (in particular head and neck squamous cell carcinoma and oropharyngeal cancer), lung cancer (in particular non-small cell lung cancer), lymphoma (in particular non-Hodgkin's lymphoma), melanoma, oral cancer, thyroid cancer, urothelial cancer or uterine cancer. In another embodiment, the cancer is selected from head and neck cancer (in particular head and neck squamous cell carcinoma and oropharyngeal cancer), lung cancer (in particular non-small cell lung cancer), urothelial cancer, melanoma or cervical cancer.

In one embodiment, the human has a solid tumor. In one embodiment, the solid tumor is advanced solid tumor. In one embodiment, the cancer is selected from head and neck cancer, squamous cell carcinoma of the head and neck (SCCHN or HNSCC), gastric cancer, melanoma, renal cell carcinoma (RCC), esophageal cancer, non-small cell lung carcinoma, prostate cancer, colorectal cancer, ovarian cancer and pancreatic cancer. In one embodiment, the cancer is selected from the group consisting of: colorectal cancer, cervical cancer, bladder cancer, urothelial cancer, head and neck cancer, melanoma, mesothelioma, non-small cell lung carcinoma, prostate cancer, esophageal cancer, and esophageal squamous cell carcinoma. In one aspect the human has one or more of the following: SCCHN, colorectal cancer, esophageal cancer, cervical cancer, bladder cancer, breast cancer, head and neck cancer, ovarian cancer, melanoma, renal cell carcinoma (RCC), esophageal squamous cell carcinoma, non-small cell lung carcinoma, mesothelioma (e.g. pleural malignant mesothelioma), and prostate cancer. In another aspect the human has a liquid tumor such as diffuse large B cell lymphoma (DLBCL), multiple myeloma, chronic lymphoblastic leukemia, follicular lymphoma, acute myeloid leukemia and chronic myelogenous leukemia.

In some embodiments, the cancer is an advanced cancer. In some embodiments, the cancer is a metastatic cancer. In some embodiments, the cancer is a recurrent cancer (e.g. a recurrent gynecological cancer such as recurrent epithelial ovarian cancer, recurrent fallopian tube cancer, recurrent primary peritoneal cancer, or recurrent endometrial cancer). In one embodiment, the cancer is recurrent or advanced.

In various embodiments, the method of the invention is employed as a first, second, third or later line of treatment. A line of treatment refers to a place in the order of treatment with different medications or other therapies received by a patient. First line therapy regimens are treatments given first, whereas second- or third-line therapy is given after the first line therapy or after the second line therapy, respectively. Therefore, first line therapy is the first treatment for a disease or condition. In patients with cancer, first line therapy, sometimes referred to as primary therapy or primary treatment, can be surgery, chemotherapy, radiation therapy, or a combination of these therapies. Typically, a patient is given a subsequent chemotherapy regimen (second or third line therapy), either because the patient did not show a positive clinical outcome or only showed a sub-clinical response to a first or second line therapy or showed a positive clinical response but later experienced a relapse, sometimes with disease now resistant to the earlier therapy that elicited the earlier positive response.

In some embodiments, the treatment of cancer is first line treatment of cancer. In one embodiment, the treatment of cancer is second line treatment of cancer. In some embodiments, the treatment is third line treatment of cancer. In some embodiments, the treatment is fourth line treatment of cancer. In some embodiments, the treatment is fifth line treatment of cancer. In some embodiments, prior treatment to said second line, third line, fourth line or fifth line treatment of cancer comprises one or more of radiotherapy, chemotherapy, surgery or radiochemotherapy.

In one embodiment, the prior treatment comprises treatment with diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; nivolumab; FOLFOX; capecitabine; FOLFIRI; bevacizumab; atezolizumab; selicrelumab; obinotuzumab or any combinations thereof. In one embodiment, prior treatment to said second line treatment, third line, fourth line or fifth line treatment of cancer comprises ipilimumab and nivolumab. In one embodiment, prior treatment to said second line treatment, third line, fourth line or fifth line treatment of cancer comprises FOLFOX, capecitabine, FOLFIRI/bevacizumab and atezolizumab/selicrelumab. In one embodiment, prior treatment to said second line treatment, third line, fourth line or fifth line treatment of cancer comprises carboplatin/Nab-paclitaxel. In one embodiment, prior treatment to said second line treatment, third line, fourth line or fifth line treatment of cancer comprises nivolumab and electrochemotherapy. In one embodiment, prior treatment to said second line treatment, third line, fourth line or fifth line treatment of cancer comprises radiotherapy, cisplatin and carboplatin/paclitaxel.

In one embodiment, the methods of the present invention further comprise administering at least one neo-plastic agent or cancer adjuvant to said human. The methods of the present invention may also be employed with other therapeutic methods of cancer treatment.

Typically, any anti-neoplastic agent or cancer adjuvant that has activity versus a tumor, such as a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10th edition (Dec. 5, 2014), Lippincott Williams & Wilkins Publishers.

In one embodiment, the human has previously been treated with one or more different cancer treatment modalities. In some embodiments, at least some of the patients in the cancer patient population have previously been treated with one or more therapies, such as surgery, radiotherapy, chemotherapy or immunotherapy. In some embodiments, at least some of the patients in the cancer patient population have previously been treated with chemotherapy (e.g. platinum-based chemotherapy). For example, a patient who has received two lines of cancer treatment can be identified as a 2 L cancer patient (e.g. a 2 L NSCLC patient). In some embodiments, a patient has received two lines or more lines of cancer treatment (e.g. a 2 L+ cancer patient such as a 2 L+ endometrial cancer patient). In some embodiments, a patient has not been previously treated with an antibody therapy, such as an anti-PD-1 therapy. In some embodiments, a patient previously received at least one line of cancer treatment (e.g. a patient previously received at least one line or at least two lines of cancer treatment). In some embodiments, a patient previously received at least one line of treatment for metastatic cancer (e.g. a patient previously received one or two lines of treatment for metastatic cancer).

The HER2 Fcab-drug conjugates according to the invention preferably exhibit an advantageous biological activity which can easily be demonstrated in enzyme assays and animal experiments, as described in the examples. In such enzyme-based assays, the HER2 Fcab-drug conjugates according to the invention preferably exhibit and cause an inhibiting effect, which is usually documented by IC₅₀ values in a suitable range, preferably in the micromolar range and more preferably in the nanomolar range.

The HER2 Fcab-drug conjugates of the present invention can be used for the preparation of pharmaceutical preparations, in particular by non-chemical methods. In this case, they are brought into a suitable dosage form together with at least one solid, liquid and/or semi-liquid excipient or adjuvant and optionally in combination with one or more further active compound(s).

Thus, the invention further relates to a pharmaceutical preparation comprising HER2 Fcab-drug conjugate according to the present invention.

In another embodiment of the present invention this pharmaceutical preparation comprises further excipients and/or adjuvants. Additionally, another embodiment according to the present invention is a pharmaceutical preparation which comprises at least one HER2 Fcab-drug conjugate according to the present invention and at least one further medicament active compound.

The invention further relates to a process for the preparation of a pharmaceutical preparation, characterised in that a HER2 Fcab-drug conjugate according to the present invention is brought into a suitable dosage form together with a solid, liquid or semi-liquid excipient or adjuvant.

The pharmaceutical preparations according to the invention can be used as medicaments in human or veterinary medicine and can be used in the therapeutic treatment of the human or animal body and in the combating of the above-mentioned diseases. The patient or host can belong to any mammal species, for example a primate species, particularly humans; rodents, including mice, rats and hamsters; rabbits; horses, cattle, dogs, cats, etc. Animal models are of interest for experimental investigations, where they provide a model for the treatment of a human disease. They can furthermore be used as diagnostic agents or as reagents.

Suitable carrier substances are organic or inorganic substances which are suitable for enteral (for example oral), parenteral or topical administration and do not react with the novel compounds, for example water, vegetable oils (such as sunflower oil or cod-liver oil), benzyl alcohols, polyethylene glycols, gelatine, carbohydrates, such as lactose or starch, magnesium stearate, talc, lanolin or Vaseline. Owing to his expert knowledge, the person skilled in the art is familiar with which adjuvants are suitable for the desired medicament formulation. Besides solvents, for example water, physiological saline solution or alcohols, such as, for example, ethanol, propanol or glycerol, sugar solutions, such as glucose or mannitol solutions, or a mixture of the said solvents, gel formers, tablet assistants and other active-ingredient carriers, it is also possible to use, for example, lubricants, stabilisers and/or wetting agents, emulsifiers, salts for influencing the osmotic pressure, antioxidants, dispersants, antifoams, buffer substances, flavours and/or aromas or flavour correctants, preservatives, solubilizers or dyes. If desired, preparations or medicaments according to the invention may comprise one or more further active compounds, for example one or more vitamins.

If desired, preparations or medicaments according to the invention may comprise one or more further active compounds and/or one or more action enhancers (adjuvants).

The terms “pharmaceutical formulation” and “pharmaceutical preparation” are used as synonyms for the purposes of the present invention.

As used here, “pharmaceutically tolerated” relates to medicaments, precipitation reagents, excipients, adjuvants, stabilisers, solvents and other agents which facilitate the administration of the pharmaceutical preparations obtained therefrom to a mammal without undesired physiological side effects, such as, for example, nausea, dizziness, digestion problems or the like.

In pharmaceutical preparations for parenteral administration, there is a requirement for isotonicity, euhydration and tolerability and safety of the formulation (low toxicity), of the adjuvants employed and of the primary packaging. Surprisingly, the HER2 Fcab-drug conjugates according to the present invention preferably have the advantage that direct use is possible and further purification steps for the removal of toxicologically unacceptable agents, such as, for example, high concentrations of organic solvents or other toxicologically unacceptable adjuvants, are thus unnecessary before use of the HER2 Fcab-drug conjugates according to the present invention in pharmaceutical formulations.

The invention particularly preferably also relates to pharmaceutical preparations comprising at least one HER2 Fcab-drug conjugate according to the present invention in precipitated non-crystalline, precipitated crystalline or in dissolved or suspended form, and optionally excipients and/or adjuvants and/or further pharmaceutical active compounds.

The HER2 Fcab-drug conjugates according to the present invention preferably enable the preparation of highly concentrated formulations without unfavourable, undesired aggregation of the HER2 Fcab-drug conjugates according to the invention occurring. Thus, ready-to-use solutions having a high active-ingredient content can be prepared with the aid of HER2 Fcab-drug conjugates according to the present invention with aqueous solvents or in aqueous media.

The HER2 Fcab-drug conjugates according to the present invention can also be lyophilised and the resultant lyophilizates used, for example, for the preparation of injection preparations.

Aqueous preparations can be prepared by dissolving or suspending HER2 Fcab-drug conjugates according to the present invention in an aqueous solution and optionally adding adjuvants. To this end, defined volumes of stock solutions comprising the said further adjuvants in defined concentration are advantageously added to a solution or suspension having a defined concentration of HER2 Fcab-drug conjugates according to the present invention, and the mixture is optionally diluted with water to the pre-calculated concentration. Alternatively, the adjuvants can be added in solid form. The amounts of stock solutions and/or water which are necessary in each case can subsequently be added to the aqueous solution or suspension obtained. HER2 Fcab-drug conjugates according to the present invention according to the invention can also advantageously be dissolved or suspended directly in a solution comprising all further adjuvants.

The solutions or suspensions comprising HER2 Fcab-drug conjugates according to the invention and having a pH of 4 to 10, preferably having a pH of 5 to 9, and an osmolality of 250 to 350 mOsmol/kg can advantageously be prepared. The pharmaceutical preparation can thus be administered directly substantially without pain intravenously, intra-arterially, intra-articularly, subcutaneously or percutaneously. In addition, the preparation may also be added to infusion solutions, such as, for example, glucose solution, isotonic saline solution or Ringer's solution, which may also contain further active compounds, thus also enabling relatively large amounts of active compound to be administered.

Pharmaceutical preparations according to the invention may also comprise mixtures of a plurality of HER2 Fcab-drug conjugates according to the present invention.

The preparations according to the invention are physiologically well tolerated, easy to prepare, can be dispensed precisely and are preferably stable with respect to assay, decomposition products and aggregates throughout storage and transport and during multiple freezing and thawing processes. They can preferably be stored in a stable manner over a period of at least three months to two years at refrigerator temperature (2-8° C.) and at room temperature (23-27° C.) and 60% relative atmospheric humidity (R.H.).

For example, the HER2 Fcab-drug conjugates according to the present invention can be stored in a stable manner by drying and when necessary converted into a ready-to-use pharmaceutical preparation by dissolution or suspension. Possible drying methods are, for example, without being restricted to these examples, nitro-gen-gas drying, vacuum-oven drying, lyophilisation, washing with organic solvents and subsequent air drying, liquid-bed drying, fluidised-bed drying, spray drying, roller drying, layer drying, air drying at room temperature and further methods.

On use of preparations or medicaments according to the invention, the HER2 Fcab-drug conjugates according to the present invention are generally used analogously to known, commercially available preparations or preparations, preferably in dosages of between 0.1 and 500 mg, in particular 5 and 300 mg, per use unit. The daily dose is preferably between 0.001 and 250 mg/kg, in particular 0.01 and 100 mg/kg, of body weight. The preparation can be administered one or more times per day, for example two, three or four times per day. However, the individual dose for a patient depends on a large number of individual factors, such as, for example, on the efficacy of the particular compound used, on the age, body weight, general state of health, sex, nutrition, on the time and method of administration, on the excretion rate, on the combination with other medicaments and on the severity and duration of the particular disease.

A measure of the uptake of a medicament active compound in an organism is its bioavailability. If the medicament active compound is delivered to the organism intravenously in the form of an injection solution, its absolute bioavailability, i.e. the proportion of the pharmaceutical which reaches the systemic blood, i.e. the major circulation, in unchanged form, is 100%. In the case of oral administration of a therapeutic active compound, the active compound is generally in the form of a solid in the formulation and must therefore first be dissolved in order that it is able to overcome the entry barriers, for example the gastrointestinal tract, the oral mucous membrane, nasal membranes or the skin, in particular the stratum corneum, or can be absorbed by the body. Data on the pharmacokinetics, i.e. on the bioavailability, can be obtained analogously to the method of J. Shaffer et al., J. Pharm. Sciences, 88 (1999), 313-318.

Furthermore, medicaments of this type can be prepared by means of one of the processes generally known in the pharmaceutical art.

Medicaments can be adapted for administration via any desired suitable route, for example by the oral (including buccal or sublingual), rectal, pulmonary, nasal, topical (including buccal, sublingual or transdermal), vaginal or parenteral (including subcutaneous, intramuscular, intravenous, intradermal and in particular intra-articular) routes. Medicaments of this type can be prepared by means of all processes known in the pharmaceutical art by, for example, combining the active HER2 Fcab-drug conjugate with the excipient(s) or adjuvant(s).

Parenteral administration is preferably suitable for administration of the medicaments according to the invention. In the case of parenteral administration, intra-articular administration is particularly preferred.

The HER2 Fcab-drug conjugates according to the invention are also suitable for the preparation of medicaments to be administered parenterally having slow, sustained and/or controlled release of active compound. They are thus also suitable for the preparation of delayed-release formulations, which are advantageous for the patient since administration is only necessary at relatively large time intervals.

The medicaments adapted to parenteral administration include aqueous and non-aqueous sterile injection solutions comprising antioxidants, buffers, bacteriostatics and solutes, by means of which the formulation is rendered isotonic with the blood or synovial fluid of the recipient to be treated; as well as aqueous and non-aqueous sterile suspensions, which can comprise suspension media and thickeners. The formulations can be delivered in single-dose or multi-dose containers, for example sealed ampoules and vials, and stored in the freeze-dried (lyophilised) state, so that only the addition of the sterile carrier liquid, for example water for injection purposes, immediately before use is necessary. Injection solutions and suspensions prepared in accordance with the formulation can be prepared from sterile powders, granules and tablets.

The HER2 Fcab-drug conjugates according to the invention can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The HER2 Fcab-drug conjugates according to the invention can also be coupled to soluble polymers as targeted medicament excipients. Such polymers can encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The HER2 Fcab-drug conjugates according to the invention can furthermore be coupled to a class of biodegradable polymers which are suitable for achieving slow release of a medicament, for example polylactic acid, poly-epsilon-caprolactone, polyhydroxybutyric acid, polyorthoesters, polyacetals, polydihydroxypyrans, polycyanoacrylates, polylactic-co-glycolic acid, polymers, such as conjugates between dextran and methacrylates, polyphosphoesters, various polysaccharides and polyamines and poly-e-caprolactone, albumin, chitosan, collagen or modified gelatine and crosslinked or amphipathic block copolymers of hydrogels.

Suitable for enteral administration (oral or rectal) are, in particular, tablets, dragees, capsules, syrups, juices, drops or suppositories, and suitable for topical use are ointments, creams, pastes, lotions, gels, sprays, foams, aerosols, solutions (for example solutions in alcohols, such as ethanol or isopropanol, acetonitrile, DMF, dimethylacetamide, 1,2-propanediol or mixtures thereof with one another and/or with water) or powders. Also particularly suitable for topical uses are liposomal preparations.

In the case of formulation to give an ointment, the active compound can be employed either with a paraffinic or a water-miscible cream base. Alternatively, the active HER2 Fcab-drug conjugate can be formulated to a cream with an oil-in-water cream base or a water-in-oil base.

Medicaments adapted to transdermal administration can be delivered as independent plasters for extended, close contact with the epidermis of the recipient. Thus, for example, the active HER2 Fcab-drug conjugate can be supplied from the plaster by means of iontophoresis, as described in general terms in Pharmaceutical Research, 3 (6), 318 (1986).

It goes without saying that, besides the constituents particularly mentioned above, the medicaments according to the invention may also comprise other agents usual in the art with respect to the particular type of pharmaceutical formulation.

The HER2 Fcab-drug conjugate described herein may also be in the form of pharmaceutical formulations, pharmaceutical preparations, sets or kits.

The present invention further relates to a set (kit) consisting of separate packs of
a) an effective amount of comprising at least one HER2 Fcab-drug conjugate according to the present invention, and
b) an effective amount of a further medicament active compound.

The set comprises suitable containers, such as boxes or cartons, individual bottles, bags or ampoules. The set may, for example, comprise separate ampoules each containing an effective amount of a HER2 Fcab-drug conjugate according to the present invention and an effective amount of a further medicament active compound in dissolved or lyophilised form.

In one embodiment, the HER2 Fcab-drug conjugate according to the present invention is administered once every 2-6 weeks (e.g. 2, 3 or 4 weeks, in particular 3 weeks). In one embodiment, the HER2 Fcab-drug conjugate is administered for once every two weeks (“Q2W”). In one embodiment, the HER2 Fcab-drug conjugate is administered for once every three weeks (“Q3W”). In one embodiment, the HER2 Fcab-drug conjugate is administered for once every 6 weeks (“Q6W”). In one embodiment, the HER2 Fcab-drug conjugate is administered for Q3W for 2-6 dosing cycles (e.g. the first 3, 4, or 5 dosing cycles, in particular, the first 4 dosing cycles).

In certain embodiments, the cancer to be treated is HER2 positive. For example, in certain embodiments, the cancer to be treated exhibits HER2+ expression (e.g., high HER2 expression). Methods of detecting a biomarker, such as HER2 for example, on a cancer or tumor, are routine in the art and are contemplated herein. Non-limiting examples include immunohistochemistry, immunofluorescence and fluorescence activated cell sorting (FACS). In some embodiments, subjects or patients with HER2 high cancer are treated by intravenously administering anti-HER2 Fcab-drug conjugate at a dose of about 1200 mg Q2W. In some embodiments, subjects or patients with HER2 high cancer are treated by intravenously administering HER2 Fcab-drug conjugate at a dose of about 1800 mg Q3W. In some embodiments, subjects or patients with HER2 high cancer are treated by intravenously administering HER2 Fcab-drug conjugate at a dose of about 2100 mg Q3W. In some embodiments, subjects or patients with HER2 high cancer are treated by intravenously administering HER2 Fcab-drug conjugate at a dose of about 2400 mg Q3W. In some embodiments, subjects or patients with HER2 high cancer are treated by intravenously administering HER2 Fcab-drug conjugate n at a dose of about 15 mg/kg Q3W.

In certain embodiments, the cancer to be treated has elevated levels of adenosine in the tumor microenvironment.

In certain embodiments, the dosing regimen comprises administering the anti-HER2 Fcab-drug conjugate, at a dose of about 0.01-3000 mg (e.g. a dose about 0.01 mg; a dose about 0.08 mg; a dose about 0.1 mg; a dose about 0.24 mg; a dose about 0.8 mg; a dose about 1 mg; a dose about 2.4 mg; a dose about 8 mg; a dose about 10 mg; a dose about 20 mg; a dose about 24 mg; a dose about 30 mg; a dose about 40 mg; a dose about 48 mg; a dose about 50 mg; a dose about 60 mg; a dose about 70 mg; a dose about 80 mg; a dose about 90 mg; a dose about 100 mg; a dose about 160 mg; a dose about 200 mg; a dose about 240 mg; a dose about 300 mg; a dose about 400 mg; a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; a dose about 2000 mg; a dose about 2100 mg; a dose about 2200 mg; a dose about 2300 mg; a dose about 2400 mg; a dose about 2500 mg; a dose about 2600 mg; a dose about 2700 mg; a dose about 2800 mg; a dose about 2900 mg; or a dose about 3000 mg). In some embodiments, the dose is a dose of about 500 mg. In some embodiments, the dose is about 1200 mg. In some embodiments, the dose is about 2400 mg. In some embodiments, the dose of the HER2 Fcab-drug conjugate is about 0.001-100 mg/kg (e.g., a dose about 0.001 mg/kg; a dose about 0.003 mg/kg; a dose about 0.01 mg/kg; a dose about 0.03 mg/kg; a dose about 0.1 mg/kg; a dose about 0.3 mg/kg; a dose about 1 mg/kg; a dose about 2 mg/kg; a dose about 3 mg/kg; a dose about 10 mg/kg; a dose about 15 mg/kg; or a dose about 30 mg/kg).

All fixed doses disclosed herein are considered comparable to the body-weight dosing based on a reference body weight of 80 kg. Accordingly, when reference is made to a fixed dose of 2400 mg, a body-weight dose of 30 mg/kg is likewise disclosed therewith.

Concurrent treatment in addition to the treatment with the HER2 Fcab-drug conjugate of the invention and considered necessary for the patient's well-being may be given at discretion of the treating physician. In some embodiments, the present invention provides methods of treating, stabilizing or decreasing the severity or progression of one or more diseases or disorders described herein comprising administering to a patient in need thereof a HER2 Fcab-drug conjugate with an additional therapy, such as chemotherapy, radiotherapy or chemoradiotherapy.

In one embodiment, diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, pemetrexed, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; tremelimumab; nivolumab; pembrolizumab; FOLFOX; capecitabine; FOLFIRI; bevacizumab; atezolizumab; selicrelumab; obinotuzumab or any combinations thereof is/are further administered.

In one embodiment, radiotherapy is further administered concurrently or sequentially with the HER2 Fcab-drug conjugate. In some embodiments, the radiotherapy is selected from the group consisting of systemic radiation therapy, external beam radiation therapy, image-guided radiation therapy, tomotherapy, stereotactic radio surgery, stereotactic body radiation therapy, and proton therapy. In some embodiments, the radiotherapy comprises external-beam radiation therapy, internal radiation therapy (brachytherapy), or systemic radiation therapy. See, e.g., Amini et al., Radiat Oncol. “Stereotactic body radiation therapy (SBRT) for lung cancer patients previously treated with conventional radiotherapy: a review” 9:210 (2014); Baker et al., Radiat Oncol. “A critical review of recent developments in radiotherapy for non-small cell lung cancer” 11(1): 115 (2016); Ko et al., Clin Cancer Res “The Integration of Radiotherapy with Immunotherapy for the Treatment of Non-Small Cell Lung Cancer” (24) (23) 5792-5806; and, Yamoah et al., Int J Radiat Oncol Biol Phys “Radiotherapy Intensification for Solid Tumors: A Systematic Review of Randomized Trials” 93(4): 737-745 (2015).

In some embodiments, the radiotherapy comprises external-beam radiation therapy, and the external bean radiation therapy comprises intensity-modulated radiation therapy (IMRT), image-guided radiation therapy (IGRT), tomotherapy, stereotactic radiosurgery, stereotactic body radiation therapy, proton therapy, or other charged particle beams.

In some embodiments, the radiotherapy comprises stereotactic body radiation therapy.

Besides the HER2 Fcab-drug conjugate according to the invention, the pharmaceutical preparations according to the invention may also comprise further medicament active compounds, for example for use in the treatment of cancer, other anti-tumor medicaments. For the treatment of the other diseases mentioned, the pharmaceutical preparations according to the invention may also, besides the HER2 Fcab-drug conjugate according to the invention, comprise further medicament active compounds which are known to the person skilled in the art in the treatment thereof.

In one embodiment, the method comprises administering a HER2 Fcab-drug conjugate of the present invention to a host in combination or alternation with an antibody. In particular subembodiments, the antibody is a therapeutic antibody. In one particular embodiment, a method of enhancing efficacy of passive antibody therapy is provided comprising administering a HER2 Fcab-drug conjugate of the present invention in combination or alternation with one or more passive antibodies. This method can enhance the efficacy of antibody therapy for treatment of abnormal cell proliferative disorders such as cancer or can enhance the efficacy of therapy in the treatment or prevention of infectious diseases. The HER2 Fcab-drug conjugate of the present invention can be administered in combination or alternation with antibodies such as rituximab, herceptin or erbitux, for example.

In another principal embodiment, a method of treating or preventing abnormal cell proliferation is provided comprising administering a HER2 Fcab-drug conjugate of the present invention to a host in need thereof substantially in the absence of another anti-cancer agent.

In another principal embodiment, a method of treating or preventing abnormal cell proliferation in a host in need thereof is provided, comprising administering a first a HER2 Fcab-drug conjugate of the present invention substantially in combination with a first anti-cancer agent to the host and subsequently administering a second HER2 Fcab-drug conjugate. In one subembodiment, the second HER2 Fcab-drug conjugate is administered substantially in the absence of another anti-cancer agent.

In another principal embodiment, a method of treating or preventing abnormal cell proliferation in a host in need thereof is provided, comprising administering a HER2 Fcab-drug conjugate of the present invention substantially in combination with a first anti-cancer agent to the host and subsequently administering a second anti-cancer agent in the absence of the HER2 Fcab-drug conjugate.

Thus, the cancer treatment disclosed here can be carried out as therapy with a HER2 Fcab-drug conjugate of the present invention or in combination with an operation, irradiation or chemotherapy. Chemotherapy of this type can include the use of one or more active compounds of the following categories of antitumour active compounds:
(i) antiproliferative/antineoplastic/DNA-damaging active compounds and combinations thereof, as used in medical oncology, such as alkylating active compounds (for example cis-platin, parboplatin, cyclophosphamide, nitrogen mustard, melphalan, chlorambucil, busulphan and nitrosoureas); antimetabolites (for example antifolates such as fluoropyrimidines such as 5-fluorouracil and tegafur, raltitrexed, methotrexate, cytosine arabinoside, hydroxyurea and gemcitabine); antitumor antibiotics (for example anthracyclines, such as adriamycin, bleomycin, doxorubicin, daunomycin, epirubicin, idarubicin, mitomycin-C, dactinomycin and mithramycin); antimitotic active compounds (for example vinca alkaloids, such as vincristine, vinblastine, vindesine and vinorelbine, and taxoids, such as taxol and taxotere); topoisomerase inhibitors (for example epipodophyllotoxins, such as etoposide and teniposide, amsacrine, topotecan, irinotecan and camptothecin) and cell-differentiating active compounds (for example all-trans-retinoic acid, 13-cis-retinoic acid and fenretinide);
(ii) cytostatic active compounds, such as anti-oestrogens (for example tamoxifen, toremifene, raloxifene, droloxifene and iodoxyfene), oestrogen receptor regulators (for example fulvestrant), anti-androgens (for example bicalutamide, flutamide, nilutamide and cyproterone acetate), LHRH antagonists or LHRH agonists (for example goserelin, leuprorelin and buserelin), progesterones (for example megestrol acetate), aromatase inhibitors (for example anastrozole, letrozole, vorazole and exemestane) and inhibitors of 5α-reductase, such as finasteride;
(iii) active compounds which inhibit cancer invasion including for example metallo-proteinase inhibitors, like marimastat, and inhibitors of urokinase plasminogen activator receptor function;
(iv) inhibitors of growth factor function, for example growth factor antibodies, growth factor receptor antibodies, for example the anti-erbb2 antibody trastuzumab [Herceptin™] and the anti-erbbl antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors, such as N-(3-chloro-4-fluorophenyl)-7-methoxy-6-(3-morpholinopropoxy) quinazolin-4-amine (gefitinib, AZD1839), N-(3-ethynylphenyl)-6,7-bis (2-methoxyethoxy) quinazolin-4-amine (erlotinib, OSI-774) and 6-acrylamido-N-(3-chloro-4-fluorophenyl)-7-(3-morpholinopropoxy) quinazolin-4-amine (CI 1033), for example inhibitors of the platelet-derived growth factor family and, for example, inhibitors of the hepatocyte growth factor family;
(v) anti-angiogenic active compounds, such as bevacizumab, angiostatin, endostatin, linomide, batimastat, captopril, cartilage derived inhibitor, genistein, interleukin 12, lavendustin, medroxypregesterone acetate, recombinant human platelet factor 4, tecogalan, thrombospondin, TNP-470, anti-VEGF monoclonal antibody, soluble VEGF-receptor chimaeric protein, anti-VEGF receptor antibodies, anti-PDGF receptors, inhibitors of integrins, tyrosine kinase inhibitors, serine/threonine kinase inhibitors, antisense oligonucleotides, antisense oligodexoynucleotides, siRNAs, anti-VEGF aptamers, pigment epithelium derived factor and compounds which have been published in the international patent applications WO 97/22596, WO 97/30035, WO 97/32856 and WO 98/13354);
(vi) vessel-destroying agents, such as combretastatin A4 and compounds which have been published in the international patent applications WO 99/02166, WO 00/40529, WO 00/41669, WO 01/92224, WO 02/04434 and WO 02/08213;
(vii) antisense therapies, for example those directed to the targets mentioned above, such as ISIS 2503, an anti-Ras antisense;
(viii) gene therapy approaches, including, for example, approaches for replacement of abnormal, modified genes, such as abnormal p53 or abnormal BRCA1 or BRCA2, GDEPT approaches (gene-directed enzyme pro-drug therapy), such as those which use cytosine deaminase, thymidine kinase or a bacterial nitroreductase enzyme, and approaches which increase the tolerance of a patient to chemotherapy or radiotherapy, such as multi-drug resistance therapy; and
(ix) immunotherapy approaches, including, for example, ex-vivo and in-vivo approaches for increasing the immunogenicity of tumor cells of a patient, such as transfection with cytokines, such as interleukin 2, interleukin 4 or granulocyte macrophage colony stimulating factor, approaches for decreasing T-cell anergy, approaches using transfected immune cells, such as cytokine-transfected dendritic cells, approaches for use of cytokine-transfected tumor cells and approaches for use of anti-idiotypic antibodies
(x) chemotherapeutic agents including for example abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, epirubicin, epoetin alfa, estramustine, etoposide, exemestane, filgrastim, floxuridine, fludarabine, fluorouracil, fulvestrant and gemcitabine.

The medicaments from table 1 can preferably, but not exclusively, be combined with the HER2 Fcab-drug conjugates of the present invention.

TABLE 1

| | | |
|---|---|---|
| Alkylating active | Cyclophosphamide | Lomustine |
| compounds | Busulfan | Procarbazine |
| | Ifosfamide | Altretamine |
| | Melphalan | Estramustine phosphate |
| | Hexamethylmelamine | Mechloroethamine |
| | Thiotepa | Streptozocin |
| | chloroambucil | Temozolomide |
| | Dacarbazine | Semustine |
| | Carmustine | |
| Platinum active | Cisplatin | Carboplatin |
| compounds | Oxaliplatin | ZD-0473 (AnorMED) |
| | Spiroplatin | Lobaplatin (Aetema) |
| | Carboxyphthalatoplatinum | Satraplatin (Johnson |
| | Tetraplatin | Matthey) |
| | Ormiplatin | BBR-3464 |
| | Iproplatin | (Hoffrnann-La Roche) |
| | | SM-11355 (Sumitomo) |
| | | AP-5280 (Access) |
| Antimetabolites | Azacytidine | Tomudex |
| | Gemcitabine | Trimetrexate |
| | Capecitabine | Deoxycoformycin |
| | 5-Fluorouracil | Fludarabine |
| | Floxuridine | Pentostatin |
| | 2-Chlorodesoxyadenosine | Raltitrexed |
| | 6-Mercaptopurine | Hydroxyurea |
| | 6-Thioguanine | Decitabine (SuperGen) |
| | Cytarabine | Clofarabine (Bioenvision) |
| | 2-Fluorodesoxycytidine | Irofulven (MGI Pharrna) |
| | Methotrexate | DMDC (Hoffmann-La Roche) |
| | Idatrexate | Ethynylcytidine (Taiho) |
| Topoisomerase | Amsacrine | Rubitecan (SuperGen) |
| inhibitors | Epirubicin | Exatecan mesylate (Daiichi) |
| | Etoposide | Quinamed (ChemGenex) |
| | Teniposide or mitoxantrone | Gimatecan (Sigma- Tau) |
| | Irinotecan (CPT-11) | Diflomotecan (Beaufour- |
| | 7-ethyl-10- | Ipsen) |
| | hydroxycamptothecin | TAS-103 (Taiho) |
| | Topotecan | Elsamitrucin (Spectrum) |
| | Dexrazoxanet (TopoTarget) | J-107088 (Merck & Co) |
| | Pixantrone (Novuspharrna) | BNP-1350 (BioNumerik) |
| | Rebeccamycin analogue | CKD-602 (Chong Kun Dang) |
| | (Exelixis) | KW-2170 (Kyowa Hakko) |
| | BBR-3576 (Novuspharrna) | |
| Antitumour | Dactinomycin (Actinomycin | Amonafide |
| antibiotics | D) | Azonafide |
| | Doxorubicin (Adriamycin) | Anthrapyrazole |
| | Deoxyrubicin | Oxantrazole |
| | Valrubicin | Losoxantrone |
| | Daunorubicin (Daunomycin) | Bleomycin sulfate (Blenoxan) |
| | Epirubicin | Bleomycinic acid |
| | Therarubicin | Bleomycin A |
| | Idarubicin | Bleomycin B |
| | Rubidazon | Mitomycin C |
| | Plicamycinp | MEN-10755 (Menarini) |
| | Porfiromycin | GPX-100 (Gem |
| | Cyanomorpholinodoxorubicin | Pharmaceuticals) |
| | Mitoxantron (Novantron) | |
| Antimitotic active | Paclitaxel | SB 408075 |
| compounds | Docetaxel | (GlaxoSmithKline) |
| | Colchicine | E7010 (Abbott) |
| | Vinblastine | PG-TXL (Cell Therapeutics) |
| | Vincristine | IDN 5109 (Bayer) |
| | Vinorelbine | A 105972 (Abbott) |
| | Vindesine | A 204197 (Abbott) |
| | Dolastatin 10 (NCI) | LU 223651 (BASF) |
| | Rhizoxin (Fujisawa) | D 24851 (ASTA Medica) |
| | Mivobulin (Warner-Lambert) | ER-86526 (Eisai) |
| | Cemadotin (BASF) | Combretastatin A4 (BMS) |
| | RPR 109881A (Aventis) | Isohomohalichondrin-B |
| | TXD 258 (Aventis) | (PharmaMar) |
| | Epothilone B (Novartis) | ZD 6126 (AstraZeneca) |
| | T 900607 (Tularik) | PEG-Paclitaxel (Enzon) |
| | T 138067 (Tularik) | AZ10992 (Asahi) |
| | Cryptophycin 52 (Eli Lilly) | !DN-5109 (Indena) |
| | Vinflunine (Fabre) | AVLB (Prescient |
| | Auristatin PE (Teikoku | NeuroPharma) |
| | Hormone) | Azaepothilon B (BMS) |
| | BMS 247550 (BMS) | BNP- 7787 (BioNumerik) |
| | BMS 184476 (BMS) | CA-4-prodrug (OXiGENE) |
| | BMS 188797 (BMS) | Dolastatin-10 (NrH) |
| | Taxoprexin (Protarga) | CA-4 (OXiGENE) |
| Aromatase | Aminoglutethimide | Exemestan |
| inhibitors | Letrozole | Atamestan (BioMedicines) |
| | Anastrazole | YM-511 (Yamanouchi) |
| | Formestan | |
| Thymidylate | Pemetrexed (Eli Lilly) | Nolatrexed (Eximias) |
| Synthase | ZD-9331 (BTG) | CoFactor ™ (BioKeys) |
| inhibitors | | |
| DNA antagonists | Trabectedin (PharmaMar) | Mafosfamide (Baxter |
| | Glufosfamide (Baxter | International) |
| | International) | Apaziquone (Spectrum |
| | Albumin + 32P | Pharmaceuticals) |
| | (isotope solutions) | O6-benzylguanine (Paligent) |
| | Thymectacin (NewBiotics) | |
| | Edotreotid (Novartis) | |
| Farnesyl transferase | Arglabin (NuOncology Labs) | Tipifarnib (Johnson & |
| inhibitors | Lonafarnib (Schering-Plough) | Johnson) |
| | BAY-43-9006 (Bayer) | Perillyl alcohol (DOR |
| | | BioPharma) |
| Pump inhibitors | CBT-1 (CBA Pharma) | Zosuquidar trihydrochloride |
| | Tariquidar (Xenova) | (Eli Lilly) |
| | MS-209 (Schering AG) | Biricodar dicitrate (Vertex) |
| Histone acetyl | Tacedinaline (Pfizer) | Pivaloyloxymethyl butyrate |
| transferase | SAHA (Aton Pharma) | (Titan) |
| inhibitors | MS-275 (Schering AG) | Depsipeptide (Fujisawa) |
| Metalloproteinase | Neovastat (Aeterna | CMT -3 (CollaGenex) |
| inhibitors | Laboratories) | BMS-275291 (Celltech) |
| Ribonucleoside | Marimastat (British Biotech) | Tezacitabine (Aventis) |
| reductase | Gallium maltolate (Titan) | Didox (Molecules for Health) |
| inhibitors | Triapin (Vion) | |
| TNF-alpha | Virulizin (Lorus Therapeutics) | Revimid (Celgene) |
| agonists/ | CDC-394 (Celgene) | |
| antagonists | | |
| Endothelin-A | Atrasentan (Abbot) | YM-598 (Yamanouchi) |
| receptor antagonists | ZD-4054 (AstraZeneca) | |
| Retinoic acid | Fenretinide (Johnson & | Alitretinoin (Ligand) |
| receptor agonists | Johnson) | |
| | LGD-1550 (ligand) | |
| Immunomodulators | Interferon | Dexosome therapy (Anosys) |
| | Oncophage (Antigenics) | Pentrix (Australian Cancer |
| | GMK (Progenics) | Technology) |
| | Adenocarcinoma vaccine | JSF-154 (Tragen) |
| | (Biomira) | Cancer vaccine (Intercell) |
| | CTP-37 (AVI BioPharma) | Norelin (Biostar) |
| | JRX-2 (Immuno-Rx) | BLP-25 (Biomira) |
| | PEP-005 (Peplin Biotech) | MGV (Progenics) |
| | Synchrovax vaccines (CTL | !3-Alethin (Dovetail) |
| | Immuno) | CLL-Thera (Vasogen) |
| | Melanoma vaccines (CTL | |
| | Immuno) | |
| | p21-RAS vaccine (GemVax) | |
| Hormonal and | Oestrogens | Prednisone |
| antihormonal active | Conjugated oestrogens | Methylprednisolone |
| compounds | Ethynyloestradiol | Prednisolone |
| | Chlorotrianisene | Aminoglutethimide |
| | Idenestrol | Leuprolide |
| | Hydroxyprogesterone | Goserelin |
| | caproate | Leuporelin |
| | Medroxyprogesterone | Bicalutamide |
| | Testosterone | Flutamide |
| | Testosterone propionate | Octreotide |
| | Fluoxymesterone | Nilutamide |
| | Methyltestosterone | Mitotan |
| | Diethylstilbestrol | P-04 (Novogen) |
| | Megestrol | 2-Methoxyoestradiol (En _- |
| | Tamoxifen | treMed) |
| | Toremofin | Arzoxifen (Eli Lilly) |
| | Dexamethasone | |
| Photodynamic | Talaporfin (Light Sciences) | Pd bacteriopheophorbide |
| active compounds | Theralux (Theratechnologies) | (Yeda) |
| | Motexafin-Gadolinium | Lutetium texaphyrin |
| | (Pharmacyclics) | (Pharmacyclics) |
| | | Hypericin |
| Tyrosine kinase | Imatinib (Novartis) | Kahalide F (PharmaMar) |
| inhibitors | Leflunomide(Sugen/Pharmacia) | CEP- 701 (Cephalon) |
| | ZDI839 (AstraZeneca) | CEP-751 (Cephalon) |
| | Erlotinib (Oncogene Science) | MLN518 (Millenium) |
| | Canertjnib (Pfizer) | PKC412 (Novartis) |
| | Squalamine (Genaera) | Phenoxodiol O |
| | SU5416 (Pharmacia) | Trastuzumab (Genentech) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | Vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | EKB-569 (Wyeth) | |
| Various other active | SR-27897 (CCK-A inhibitor, | BCX-1777 (PNP inhibitor, |
| compounds | Sanofi-Synthelabo) | BioCryst) |
| | Tocladesine (cyclic AMP | Ranpirnase (ribonuclease |
| | agonist, Ribapharm) | stimulant, Alfacell) |
| | Alvocidib (CDK inhibitor, | Galarubicin (RNA synthesis |
| | Aventis) | inhibitor, Dong-A) |
| | CV-247 (COX-2 inhibitor, Ivy | Tirapazamine (reducing |
| | Medical) | agent, SRI International) |
| | P54 (COX-2 inhibitor, | N-Acetylcysteine |
| | Phytopharm) | (reducing agent, |
| | CapCell ™ (CYP450 | Zambon) |
| | stimulant, Bavarian Nordic) | R-Flurbiprofen (NF-kappaB |
| | GCS-IOO (gal3 antagonist, | inhibitor, Encore) |
| | GlycoGenesys) | 3CPA (NF-kappaB inhibitor, |
| | G17DT immunogen (gastrin | Active Biotech) |
| | inhibitor, Aphton) | Seocalcitol (vitamin D |
| | Efaproxiral (oxygenator, | receptor agonist, Leo) |
| | Allos Therapeutics) | 131-I-TM-601 (DNA |
| | PI-88 (heparanase inhibitor, | antagonist, TransMolecular) |
| | Progen) | Eflornithin (ODC inhibitor, |
| | Tesmilifen (histamine | ILEX Oncology) |
| | antagonist, YM BioSciences) | Minodronic acid (osteoclast |
| | Histamine (histamine H2 | inhibitor, |
| | receptor agonist, Maxim) | Yamanouchi) |
| | Tiazofurin (IMPDH inhibitor, | Indisulam (p53 stimulant, |
| | Ribapharm) | Eisai) |
| | Cilengitide (integrin antagonist, | Aplidin (PPT inhibitor, |
| | Merck KGaA) | PharmaMar) |
| | SR-31747 (IL-1 antagonist, | Rituximab (CD20 antibody, |
| | Sanofi-Synthelabo) | Genentech) |
| | CCI-779 (mTOR kinase | Gemtuzumab (CD33 |
| | inhibitor, Wyeth) | antibody, Wyeth Ayerst) |
| | Exisulind (PDE-V inhibitor, | PG2 (haematopoiesis |
| | Cell Pathways) | promoter, Pharmagenesis) |
| | CP-461 (PDE-V inhibitor, Cell | Immunol ™ (triclosan |
| | Pathways) | mouthwash, Endo) |
| | AG-2037 (GART inhibitor, | Triacetyluridine (uridine |
| | Pfizer) | prodrug, Wellstat) |
| | WX-UK1 (plasminogen | SN-4071 (sarcoma agent, |
| | activator inhibitor, Wilex) | Signature BioScience) |
| | PBI-1402 (PMN stimulant, | TransMID-107 ™ |
| | ProMetic LifeSciences) | (immunotoxin, KS Biomedix) |
| | Bortezomib (proteasome | PCK-3145 (apoptosis |
| | inhibitor, Millennium) | promoter, Procyon) |
| | SRL-172 (T-cell stimulant, | Doranidazole (apoptosis |
| | SR Pharma) | promoter, Pola) |
| | TLK-286 (glutathione-S | CHS-828 (cytotoxic agent, |
| | transferase inhibitor, Telik) | Leo) |
| | PT-100 (growth factor | trans-Retinoic acid |
| | agonist, Point Therapeutics) | (differentiator, NIH) |
| | Midostaurin (PKC inhibitor, | MX6 (apoptosis promoter, |
| | Novartis) | MAXIA) |
| | Bryostatin-1 (PKC stimulant, | Apomine (apoptosis |
| | GPC Biotech) | promoter, ILEX Oncology) |
| | CDA-II (apoptosis promoter, | Urocidin (apoptosis promoter, |
| | Everlife) | Bioniche) |
| | SDX-101 (apoptosis promoter, | Ro-31-7453 (apoptosis |
| | Salmedix) | promoter, La Roche) |
| | Ceflatonin (apoptosis | Brostallicin (apoptosis |
| | promoter, ChemGenex) | promoter, Pharmacia) |

The disclosure further provides diagnostic, predictive, prognostic and/or therapeutic methods using the HER2 Fcab-dyeg conjugate described herein. Such methods are based, at least in part, on determination of the identity of the expression level of a biomarker of interest. In particular, the amount of any one of human HER2 in a cancer patient sample can be used as a biomarker to predict whether the patient is likely to respond favorably to cancer therapy utilizing the therapeutic combination of the invention.

Thus, another embodiment of the present invention is a HER2 Fcab-label conjugate comprising the formula Fcab-(L) m-(La) n wherein:
a) Fcab comprises a HER2 Fcab,
b) L comprises a linker,
c) La comprises a label,
d) m is an integer from 1-5 and n is an integer from 1-10.

In a preferred embodiment of the present invention m is 1 to 3 and n is 1 to 5.

The invention relates also to HER2 Fcab-label conjugates in which the HER2 Fcab according to the present invention are modified by adding a label, yielding labelled HER2 Fcab conjugates. The label can be coupled to the HER2 Fcab via spacers/linkers of various lengths to reduce potential steric hindrance. The linkers can be the same as described above for the HER2 Fcab-drug conjugates according to the present invention.

The term “label” or “labelling group” refers to any detectable label. Exemplary labels include, but are not limited to isotopic labels, which may be radioactive or heavy isotopes, such as radioisotopes or radionuclides (e.g., ³H, ¹⁴C, ¹⁵N, ³⁵S, ⁸⁹Zr, ⁹⁰Y, ⁹⁹Tc, ¹¹¹In, ¹²⁵I, ¹³¹I); magnetic labels (e.g., magnetic particles); redox active moieties; optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluorophores or proteinaceous fluorophores; enzymatic groups (e.g., horseradish peroxidase, ˜-galactosidase, luciferase, alkaline phosphatase; biotinylated groups; or predetermined polypeptide epitopes recognized by a secondary reporter (e.g., leucine zipper pair sequences, binding sites for secondary antibodies, metal binding domains, epitope tags, etc.).

A preferred embodiment of the present invention is a HER2 Fcab-label conjugate of the present invention wherein the label is selected from the group consisting of an isotopic label, a magnetic label, a redox active moiety, an optical dye and an enzymatic group.

A further preferred embodiment of the present invention is a HER2 Fcab-label conjugate of the present invention wherein the label is a pHAb-dye.

A label according to the present invention can also be a tag, such as an affinity tag aiding in purification and isolation of the antibody. Non-limiting examples of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag.

Thus, a further preferred embodiment of the present invention is a HER2 Fcab-label conjugate of the present invention wherein the label is a tag.

Another embodiment of the present invention is a diagnostic composition containing the HER2 Fcab-label conjugates according to the present invention.

Any suitable sample can be used for the method. Non-limiting examples of such include one or more of a serum sample, plasma sample, whole blood, pancreatic juice sample, tissue sample, tumor lysate or a tumor sample, which can be an isolated from a needle biopsy, core biopsy and needle aspirate. For example, tissue, plasma or serum samples are taken from the patient before treatment and optionally on treatment with the therapeutic combination of the invention. The expression levels obtained on treatment are compared with the values obtained before starting treatment of the patient. The information obtained may be prognostic in that it can indicate whether a patient has responded favorably or unfavorably to cancer therapy.

It is to be understood that information obtained using the diagnostic assays described herein may be used alone or in combination with other information, such as, but not limited to, expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient, and the like. In a particular aspect, the expression level can be used in a diagnostic panel each of which contributes to the final diagnosis, prognosis, or treatment selected for a patient.

Any suitable method can be used to measure the biomarker protein or other suitable read-outs for biomarker levels, respectively, examples of which are described herein and/or are well known to the skilled artisan.

In some embodiments, determining the biomarker level comprises determining the biomarker expression. In some embodiments, the biomarker level is determined by the biomarker protein concentration in a patient sample, e.g., with biomarker specific ligands, such as antibodies or specific binding partners. The binding event can, e.g., be detected by competitive or non-competitive methods, including the use of a labeled ligand or biomarker specific moieties, e.g., antibodies, or labeled competitive moieties, including a labeled biomarker standard, which compete with labeled proteins for the binding event. If the biomarker specific ligand is capable of forming a complex with the biomarker, the complex formation can indicate biomarker expression in the sample. In various embodiments, the biomarker protein level is determined by a method comprising quantitative western blot, multiple immunoassay formats, ELISA, immunohistochemistry, histochemistry, or use of FACS analysis of tumor lysates, immunofluorescence staining, a bead-based suspension immunoassay, Luminex technology, or a proximity ligation assay. In one embodiment, the biomarker expression is determined by immunohistochemistry using one or more primary antibodies that specifically bind the biomarker.

However, in a preferred embodiment of the present invention the HER2 Fcab-label conjugate according to the present invention is used to determine the expression of HER2 protein in cells, organoids, serum sample, plasma sample, whole blood, pancreatic juice sample, tissue sample, tumor lysate or a tumor sample.

In one embodiment, the efficacy of the therapeutic combination of the invention is predicted by means of HER2 expression in tumor samples.

This disclosure also provides a kit for determining if the combination of the invention is suitable for therapeutic treatment of a cancer patient, comprising means for determining a protein level of HER2, in a sample isolated from the patient and instructions for use In one aspect of the invention, the determination of a high HER2 level indicates increased PFS or OS when the patient is treated with the HER2 Fcab-drug conjugate of the invention. In one embodiment of the kit, the means for determining the biomarker protein level are antibodies with specific binding to the biomarker.

### BRIEF DESCRIPTION OF THE FIGURES

FIG. 1 shows a conceptual representation of the advantages of Fcab-drug conjugates over other antibody-fragment based drug conjugates (VHH¹³⁻¹⁵, scFv^{9,10} Fab^{7,8}) and conventional IgG-based ADCs⁴.

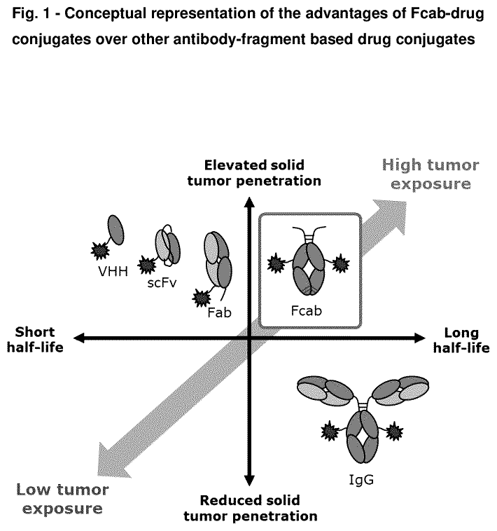

FIG. 2 shows cellular uptake data of Fcab-pHAb dye conjugates (FS-pHAb, S5-pHAb, S19-pHAb), T-IgG-pHAb and T-Fab-pHAb reference constructs and huFc-pHAb negative control on different HER2 positive (SKBR-3, HCC-1954, BT-474) and HER2 negative (MDA-MB-468) cell lines. (A) Linearly increasing (S5-pHAb, T-IgG-pHAb) and decelerating (FS-pHAb) cellular uptake is shown. Intracellular accumulation was monitored for 24 h at 100 nM in triplicates and fluorescence intensity was normalized to cell number and the pHAb-dye DOL value of each construct. Intracellular accumulation rates were derived by linear fittings. (B) Relative intracellular accumulation (±SD) refers to the highest normalized intracellular accumulation rate: S5-pHAb on SKBR-3 cells. Cell lines were selected on the basis of HER2 expression levels, with highest expression in SKBR-3, followed by HCC-1954 and BT-474.³⁸

FIG. 3 shows Fcab conjugation sites and linker-drug structures. (A) Fcab crystal structure (PDB: 5JIH, STAB19²³) is shown in cartoon representation with transparent surface. Conjugation site Q295 for mTG and mutated D265 are depicted as sticks and highlighted in blue and orange. Amino acids of N-terminal hinge region as well as LLQGA tags are not shown in crystal structure. Engineered amino acids in C_{H}3 AB and EF loop forming the HER2 paratope are marked in red. Mutations are described using EU numbering. (B) Val-Cit-MMAE cleavable linker-drug possessing either a Gly₃ handle for mTG conjugation (1) or a mc handle for cysteine conjugation (2).

FIG. 4 shows in vitro cell viability data. (A) Fcab-MMAE conjugates (red) as well as Trastuzumab-based reference MMAE conjugates (black) and huFc-based negative controls (grey) were tested on HER2 expressing SKBR-3 and HCC-1954 cell lines. Each data point in the graph represents the IC₅₀ value from an individual experiment. Bars represent the geometric mean (±SD) calculated from individual IC₅₀. Constructs were incubated on cells at 37° C. for 4 days before cell viability was measured. Unconjugated parent molecules did not show cytotoxicity under assay conditions. As expected, all conjugate constructs showed only little cytotoxic effects at higher concentrations on MDA-MB-468 HER2 negative cells (Figure S17). (B) Correlation between IC₅₀ value on SKBR-3 cells and HER2 dissociation constant (K_{D}) for DAR 2.0-2.2 Fcab-drug conjugates. (C) Exemplary viability plot of SKBR-3 cells treated with MMAE conjugates.

FIG. 5 shows a 3D tumor spheroid penetration model (A) Representative confocal microscopy images comparing high affinity versus low affinity distribution of 50 kDa pHAb-dye labeled antibody fragments in HER2 positive BT-474 and HER2 negative HCC-1937 tumor cell spheroids. (B) Representative confocal microscopy images comparing distribution of 50 kDa pHAb-dye labeled antibody fragments versus corresponding 150 kDa IgG variants in BT-474 and HCC-1937 tumor cell spheroids. (C) Radial profile plot derived from confocal microscopy images depicting semiquantitatively the penetration depth. Solid line represents the mean (n=8 spheroids/group) with SD depicted as dotted lines. (D) Mean penetration distance (±SD) of 50 kDa antibody fragments and corresponding 150 kDa IgG variants in BT-474 spheroids calculated from radial profile plots (n=8 spheroids/group). Statistical analysis performed using unpaired, two-tailed t-test, *** denoted P<0.001. Spheroids were grown from 2,000 cells for 96 h, incubated for 24 h with 50 nM pHAb-dye labeled constructs and intracellular accumulated pHAb-dye was imaged with a laser scanning confocal microscope (20×). Images were taken at spheroid diameter 341±3 μm and spheroid depth 62±3 μm.

FIG. 6 shows the purification process of Fcab FS antibody fragments by protein A for. (A) ÄKTA Xpress (HiTrap™ MabSelect SuRe™ 5 mL and HiPrep™ 26/10 desalting column) chromatogram showing protein peak after elution from Protein A column (50 mM acetic acid (HOAc), pH 3.2) and a second protein peak after a subsequent buffer change step. (B) SDS-PAGE analysis of reduced and not-reduced Expi293F supernatant, protein A flow through and purified FS. 4-12% Bis-Tris Gel (Invitrogen™), MES SDS running buffer (1×), 40 min at 200 V, stained with InstantBlue™ (Coomassie-based) for 2 h, marker: Precision Plus Protein™ Unstained Standards (BioRad).

FIG. 7 shows the purification process of His-tagged T-Fab antibody fragments by immobilized metal affinity chromatography (IMAC). A) ÄKTA Pure (1 mL HisTrap™ HP column, GE Healthcare) chromatogram showing eluted protein fractions by increasing concentrations of imidazole. (B) SDS-PAGE analysis of not-reduced and reduced pooled peaks and mixed fractions. 4-12% Bis-Tris Gel (Invitrogen™), MES SDS running buffer (1×), 40 min at 200 V, stained with InstantBlue™ (Coomassie-based) for 2 h, marker: Precision Plus Protein™ Unstained Standards (BioRad).

FIG. 8 summarizes the yields of purified proteins. Fcabs and control constructs per volume Expi-293F expression culture. Fcabs are marked red and control constructs are marked grey. Variants that contain a D265C mutation are marked with orange lines. D265C mutants expressed worse than comparable constructs lacking this mutation.

FIG. 9 shows the not-reduced and reduced purified huFc and Fcab variants. The bands of not-reduced constructs appear around the expected 50 kDa. When reduced, monomeric heavy chains appear at approx. 30 kDa. Higher apparent molecular weights of STAB5 variants (#5-10) compared to huFc or STAB19 variants (1-4, 11-12) are caused by an additional artificial NVS glycosylation site in the engineered C_{H}3 AB-loop of STAB5 which was also reported by Traxlmayr et al.⁵² 4-12% Bis-Tris Gel (Invitrogen™), MES SDS running buffer (1×), 40 min at 200 V, stained with InstantBlue™ (Coomassie-based) for 2 h, marker: Precision Plus Protein™ Unstained Standards (BioRad).

FIG. 10 shows analytical SE-HPLC chromatograms (Abs. 214 nm) of purified Fcabs and controls after a freeze-thaw cycle. Single peaks show high monomeric content and the absence of significant quantities of aggregates.

FIG. 11 shows the thermal stability of Fcabs and huFc control molecules. The first derivative of thermal unfolding curves (A) as well as the unfolding transition midpoints (Tₘ) (B) are shown. To determine thermal unfolding, Fcabs and huFc (PBS pH 6.3) were loaded into nanoDSF grade standard capillaries which were then transferred into a Prometheus NT.PLEX nanoDSF (NanoTemper Technologies) instrument. Samples were subjected to a linear thermal ramp from 20° C. to 95° C. at a slope of 1° C./min with simultaneous recording of fluorescence at 350 and 330 nm. Unfolding transition midpoints (Tₘ) were determined from the first derivative of the fluorescence ratio 350 nm/330 nm. All samples were measured in duplicates.

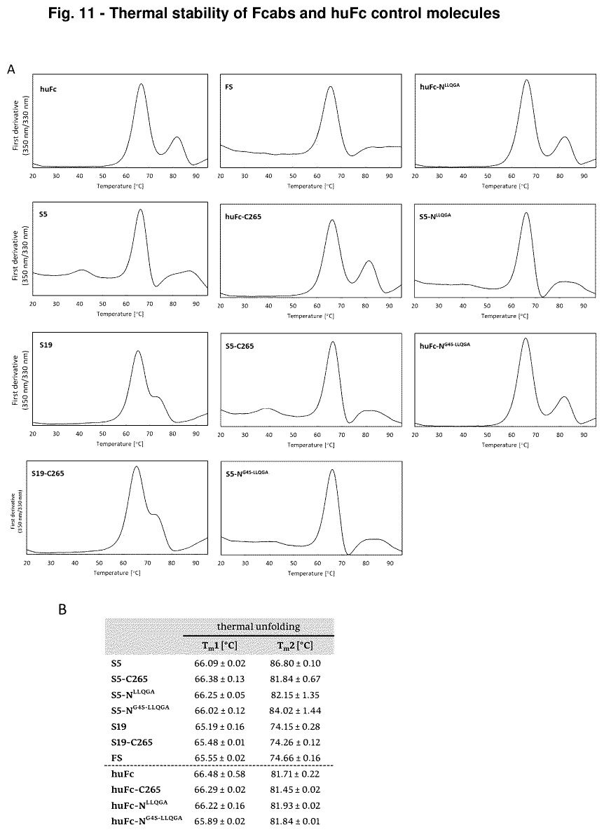

FIG. 12 shows the LC-MS analysis which confirms the identity of Fcabs and huFc controls. Mass variations between calculated and observed masses account for glycosylation patterns and standard measurement deviations. Only the most intense glycosylation patterns are listed. S5-N^{LLQGA} and huFc-N^{LLQGA} are partially O-glycosylated due to a potential O-glycosylation site (LLQGATCPPCP . . . ) generated by genetically introduced N-terminal LLQGA-tag. All STAB5 variants carry an additional Man5 glycosylation which is probably located at the artificial NVS glycosylation site in the engineered C_{H}3 AB-loop. This artificial glycosylation site was also reported by Traxlmayr et al.⁵²

FIG. 13 shows the cellular binding analysis of Fcabs and control molecules on HER2 positive (SKBR-3, HCC-1954) and HER2 negative (MDA-MB-468) cells. Fcabs and Trastuzumab reference constructs bind selectively HER2 expressing cells while huFc binds only slightly to HCC-1954 cells. Relative order of fluorescence intensity of distinct variants on HER2 positive cells correspond to their HER2 binding affinity. Cells were incubated with 100 nM of Fcab/antibody for 60 min at 4° C., washed twice with PBS-1% BSA, incubated for 30 min with 500 nM of AF488-labeled detection antibody (Jackson ImmunoResearch) at 4° C. in darkness, washed twice with PBS-1% BSA, and finally fluorescence intensity was measured applying an Attune NxT flow cytometer (Invitrogen™).

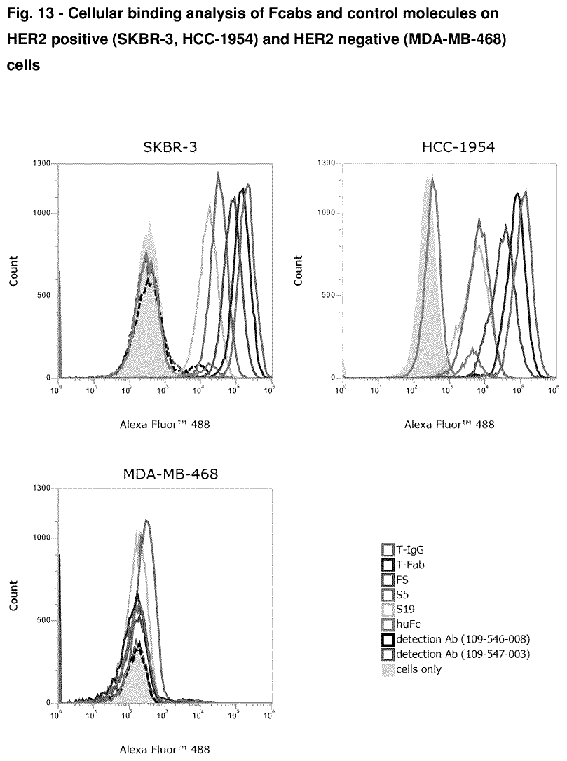

FIG. 14 shows the pHAb-dye constructs used in the experiments. (A) Structure of pHAb thiol reactive dye carrying a maleimide group 3 (Promega) which reacts with free thiol groups of cysteines. (B) Absorption and fluorescence spectra of pHAb dye in SE-HPLC running buffer (50 mM sodium phosphate, 400 mM sodium perchlorate, pH 6.3). Spectra were recorded on a microplate reader (Synergy/neo2, BioTek). (C) Generated pHAb-dye conjugates for this study. Similar degrees of labeling (DOL 1.8-2.5) were achieved by carefully adjusting the equivalents of 3 added to previously reduced proteins.

FIG. 15 shows the cellular uptake kinetics of pHAb-dye labeled constructs. (A) Intracellular accumulation time series exemplarily shown for S5-pHAb on SKBR-3 cells. Cells were incubated at 37° C., 80% humidity and 5% CO₂ with 100 nM S5-pHAb and RFP channel images (ex.: 531 nm, em.: 593 nm) were recorded every 2 h for 24 h using a Cytation 5 cell imaging reader (BioTek) equipped with DAPI and RFP filter cubes and a BioSpa 8 automated incubator (BioTek). (B) The fluorescence intensity of images is normalized to cell-number and pHAb-dye DOL of each construct and plotted over time to derive normalized intracellular accumulation rates from slopes of linearly fitted data. Subsequently, the relative intracellular accumulation can be calculated from these rates.

FIG. 16 shows the conjugation and purification strategy for Fcab-MMAE conjugates. (A) MMAE conjugates were either generated by engineered cysteine or enzymatic transglutaminase conjugation. After conjugation, excess of dehydroascorbic acid (DHA), N-acetylcysteine (NAC), mc-Val-Cit-MMAE (2) or microbial transglutaminase (mTG) and Gly₃-Val-Cit-MMAE (1) were removed by preparative SEC. (B) Purification of transglutaminase conjugated MMAE constructs by preparative SEC, exemplarily shown for S19-Q295-MMAE and huFc-Q295-MMAE. Fractions containing conjugated proteins (and non-conjugated species) were pooled, concentrated, sterile filtered and subjected to analytics. Peak intensities represent absorption at 280 nm.

FIG. 17 shows the chromatographic characterization of generated MMAE conjugates for FS-Q295-MMAE, huFc-Q295-MMAE and T-Fab-C183, C205-MMAE. (A) Analytical size exclusion SE-HPLC shows a distinct single peak demonstrating formation of monomeric drug conjugates without aggregates. Signal intensity represents absorption at 214 nm (B) Reversed phase RP-HPLC reveals conjugation of Gly₃-Val-Cit-MMAE 1 or mc-Val-Cit-MMAE 2. RP-DAR is calculated from peak areas of individual DAR species. For example, 25% relative peak area of DAR 1 species T-Fab-C183, C205-MMAE and 75% relative peak area of DAR 2 species T-Fab-C183, C205-MMAE reveals a final RP-DAR of 1.75. Signal intensity represents absorption at 214 nm. (C) Hydrophobic interaction HI-HPLC separates distinct DAR species according to their hydrophobicity. HIC-DAR can be calculated from peak areas just as RP-DAR. Moreover, relative retention times (RRT) can be calculated from HIC data to characterize the intrinsic hydrophobicity of an ADC. RRT were calculated from the elution times of the DAR 2.0 drug conjugate and the parental antibody (Ab) emphasizing the hydrophobicity added by linker-drug to each construct. Signal intensity represents absorption at 280 nm.

FIG. 18 shows the DAR determination of S19-Q295-MMAE by LC-MS. (A) Reversed phase chromatogram of reduced drug conjugate and DAR calculation. (B) Deconvoluted MS spectra used to assign RP peaks to individual heavy chain species conjugated with Gly₃-Val-Cit-MMAE (1).

FIG. 19 shows the DAR determination of huFc-Q295-MMAE by LC-MS. (A) Reversed phase chromatogram of reduced drug conjugate and DAR calculation. (B) Deconvoluted MS spectra used to assign RP peaks to individual heavy chain species conjugated with Gly₃-Val-Cit-MMAE (1).

FIG. 20 shows the kinetic HER2 binding parameters of MMAE conjugates and unconjugated parent molecules. Dissociation constants (K_{D}), on-(kₒₙ) and off-rates (k_{off}) were measured at pH 7.4 by BLI using recombinantly produced HER2. Errors are standard errors from fitting using FortéBio data analysis software 9.1. Fitting quality is characterized by R². Data is derived from BLI sensorgrams represented in FIG. 22 and FIG. 23.

FIG. 21 shows the kinetic FcRn binding parameters of MMAE conjugates and unconjugated parent molecules. Dissociation constants (K_{D}), on-(kₒₙ) and off-rates (k_{off}) were measured by BLI using recombinantly produced FcRn. Binding affinity to FcRn was determined at pH 6.0. Errors are standard errors from fitting using FortéBio data analysis software 9.1. Fitting quality is characterized by R². Data is derived from BLI sensorgrams represented in FIG. 22 and FIG. 23.

FIG. 22 shows the HER2 binding analysis of unconjugated Fcabs, Trastuzumab variants and respective MMAE conjugates via BLI. Association and dissociation were either fitted by a 1:1 global full-fit binding model or by a 1:1 global partial-dissociation model (only STAB19 variants). Fittings are shown in red. For each sensorgram, the highest concentration of analyte during association and its dilution factor are given.

FIG. 23 shows the FcRn binding analysis of unconjugated Fcabs, Trastuzumab variants and respective MMAE conjugates via BLI. Association and dissociation of analytes (1 μM; 1:2 serial diluted) were recorded at pH 6.0 and fitted by a 1:1 global partial-dissociation model. Fittings are shown in red.

FIG. 24 shows the in vitro stability evaluation for S5-MMAE conjugates in mouse and human serum. (A) Mouse serum incubation reveals MMAE release from N-terminal conjugated STAB5 variants. Contrarily, Q295 or C265 conjugated STAB5 variants show very low release of MMAE and hence excellent conjugate stability. (B) No free MMAE was detected when constructs were incubated in human serum. Free MMAE was measured via LC MS/MS after incubation in mouse and human serum at 37° C. for 96 h (n=3).

FIG. 25 shows the in vitro cytotoxicity data. (A) Exemplary viability plots of HER2 positive (SKBR-3, HCC-1954) and HER2 negative cells (MDA-MB-468) treated with serial dilutions of Fcab-drug conjugates and controls. (B) IC₅₀ values of Fcab-drug conjugates and controls derived from viability curves. Since the number of conjugated drugs and target affinity of the antibody impact cytotoxic activity, DAR values and HER2 dissociation constants (K_{D}) are listed as well.

FIG. 26 shows the formation of tumor cell spheroids. (A) Wide field images showing exemplarily tumor cell spheroid formation of 8000 HCC-1937 cells over 24 h at 37° C., 80% humidity and 5% CO₂. Wide field images were taken with an IncuCyte live-cell analysis system (Sartorius). (B) Confocal microscopy images showing 4 different BT-474 cell spheroids with reproducible size (2,000 cells were grown for 96 h at 37° C., 80% humidity and 5% CO₂). Confocal microscopy images were taken with at 20-fold magnification with a confocal laser scanning microscope TCS SP8 (Leica).

FIG. 27 shows confocal microscopy images of BT-474 tumor cell spheroids (2,000 cells grown for 96 h at 37° C., 80% humidity and 5% CO₂) incubated with 50 nM pHAb-dye labeled constructs for 24 h. Images were taken with a confocal laser scanning microscope TCS SP8 (Leica, 20 fold magnification) at spheroid diameter 341±3 μm and spheroid depth 62±3 μm. For visual comparability the brightness of images was adjusted to compensate differences resulting from distinct pHAb-dye labeling degrees. For better visualization, the contrast of all images was increased by 40%. Radial profile plots and MPD were derived from unprocessed images.

FIG. 28 shows the quantification strategy for tumor cell spheroid penetration. (A) Confocal microscopy image of BT-474 spheroid incubated with 50 nM pHAb-dye labeled T-IgG for 24 h. The picture was taken 50 μm above the bottom of the spheroid (z-position). Fluorescence of intracellular accumulated T-IgG-pHAb is shown in red. The yellow circle marks the border of the spheroid and was set manually using the radial profile plot plug-in in ImageJ.⁵⁶ The radial profile plot plug-in produces a profile plot of normalized integrated intensities around concentric circles as a function of distances from the center of the yellow circle (spheroid) (B) Brightfield image of the same spheroid. (C) Radial profile plot generated from the BT-474 spheroid by ImageJ. The fluorescence intensity profile of T-IgG-pHAb (A) is reflected in the high intensity at larger radii (border of the spheroid). Its limited distribution towards the center of the spheroid produces a sharp decrease of fluorescence intensity towards smaller radii (center of the spheroid). From this radial fluorescence profile, the mean penetration distance (MPD) can be calculated. The MPD allows to compare the spheroid penetration properties of distinct molecules. (D) Equation for the calculation of the mean penetration distance from radial profile plots (C).

Even without further embodiments, it is assumed that a person skilled in the art will be able to use the above description in the broadest scope. The preferred embodiments should therefore merely be regarded as descriptive disclosure which is absolutely not limiting in any way.

All the references cited herein are incorporated by reference in the disclosure of the invention hereby.

Although methods and materials similar or equivalent to those described herein can be used in the practice or testing of the present invention, suitable examples are described below. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again provided that the technical problem of the invention is solved. Similarly, the features of any claim can be combined with the features of one or more other claims. The present invention having been described in summary and in detail, is illustrated and not limited by the following examples.

Unless indicated otherwise, percent data denote percent by weight. All temperatures are indicated in degrees Celsius. “Conventional work-up”: water is added if necessary, the pH is adjusted, if necessary, to values between 2 and 10, depending on the constitution of the end product, the mixture is extracted with ethyl acetate or dichloromethane, the phases are separated, the organic phase is dried over sodium sulfate, filtered and evaporated, and the product is purified by chromatography on silica gel and/or by crystallisation.

Rf values on silica gel; mass spectrometry: El (electron impact ionisation): M⁺, FAB (fast atom bombardment): (M+H)⁺, THF (tetrahydrofuran), NMP (N-methlpyrrolidone), DMSO (dimethyl sulfoxide), EA (ethyl acetate), MeOH (methanol), TLC (thin-layer chromatography)

### LIST OF ABBREVIATIONS

AUC Area under the plasma drug concentration-time curve
Cₘₐₓ Maximum plasma concentration
C_{L} Clearance
CV Coefficient of variation
CYP Cytochrome P450
DMSO Dimethyl sulfoxide
F Bioavailability
fₐ Fraction absorbed
iv Intravenous
LC-MS/MS Liquid chromatography tandem mass spectrometry
LLOQ Lower limit of quantification
NC Not calculated
ND Not determined
PEG Polyethylene glycol
Pgp Permeability glycoprotein
PK Pharmacokinetic(s)
po Per os (oral)
t_{1/2} Half-life
tₘₐₓ Time at which maximum plasma concentration of drug is reached
UPLC Ultra performance liquid chromatography
Vₛₛ Volume of distribution (at steady state)
v/v Volume to volume

### EXAMPLES

### Example 1: Preparation of Fcabs and Controls

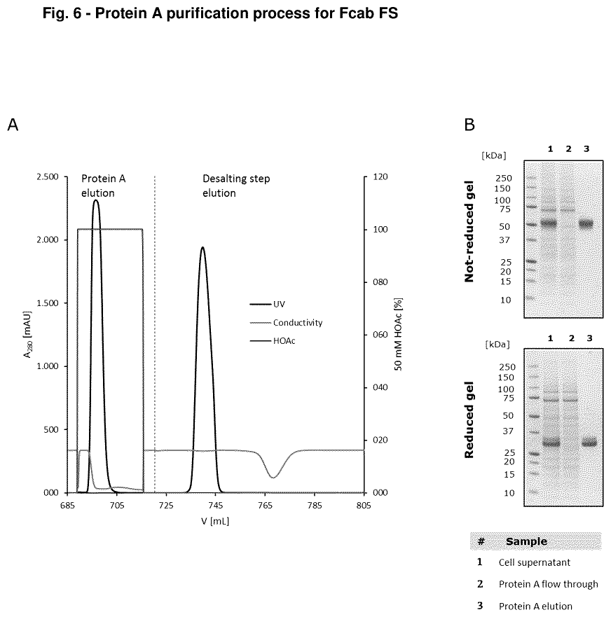

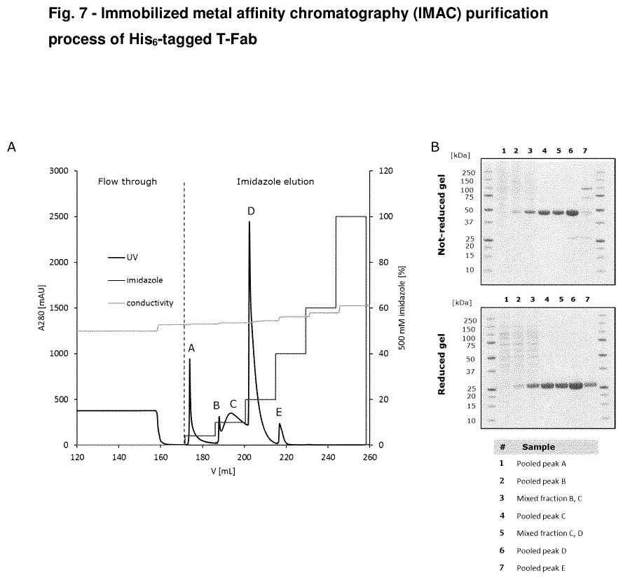

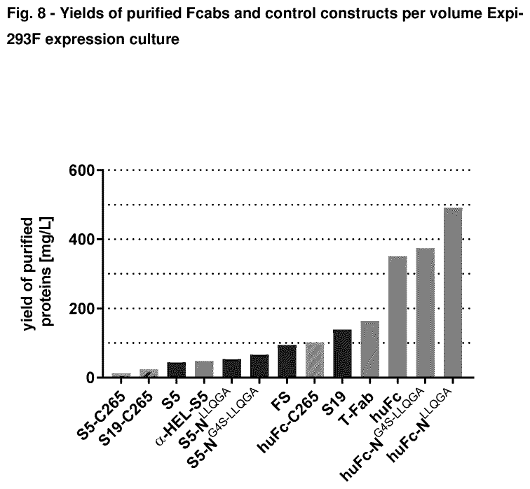

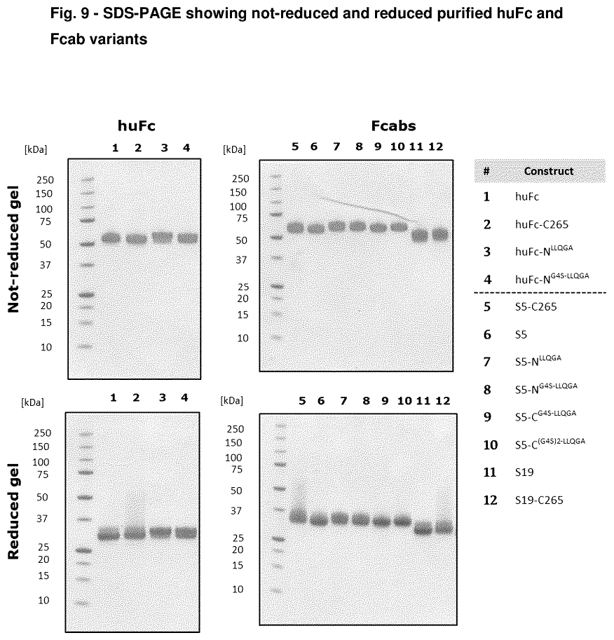

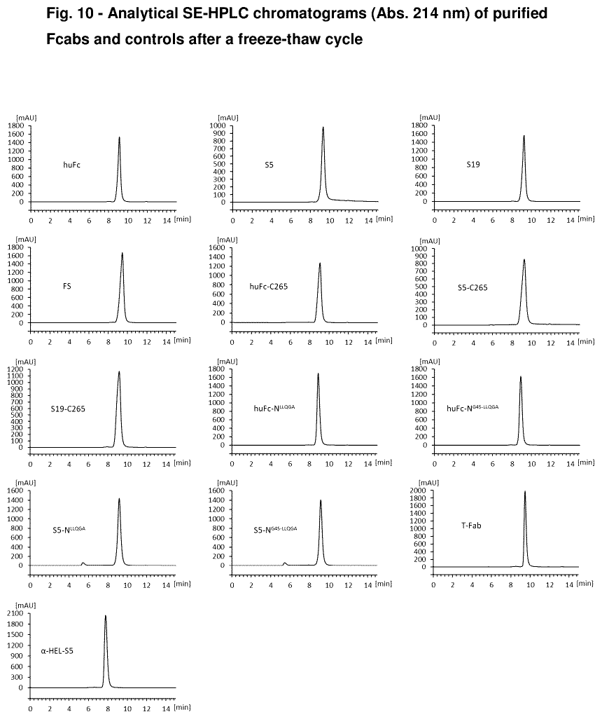

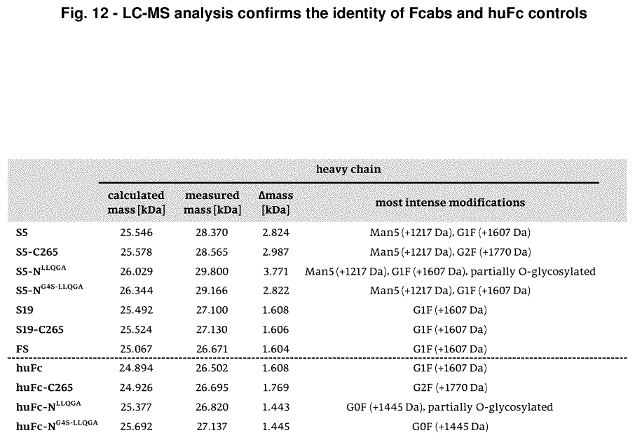

Three different Fcabs from the literature with subnanomolar to double-digit nanomolar binding affinities to HER2 were selected: STAB5, STAB19²⁷, and the clinical candidate FS102²⁴. To prepare Fcabs for the generation of ADCs, different constructs were designed (Table 1). For site-specific bioconjugation, STAB5 and STAB19 scaffolds were engineered by incorporation of a cysteine residue at position D265C²⁸ (S5-C265, S19-C265). The STAB5 scaffold was chosen for genetic fusion of N- and C-terminal transglutaminase recognition tags (LLQGA²⁹) that allow for transglutaminase-mediated bioconjugation (S5-N^{LLQGA}, S5-N^{G4S-LLQGA}, S5-C^{G4S-LLQGA}, S5-C^{(G4S)2-LLQGA)}. Moreover, an effector silencing mutation (D265A^{30,31}) was incorporated in all Fcab variants (except S5-C265, S19-C265) to avoid effects mediated by FcγRI, II, III receptor binding.³² As a control for subsequent spheroid penetration assays, a full-length 150 kDa STAB5 variant (a-HEL-S5) was designed with unrelated anti-hen egg lysozyme (HEL) Fab fragments genetically fused onto the Fcab scaffold. Moreover, native human Fc (huFc)-based negative controls (huFc, huFc-C265, huFc-N^{LLQGA}, huFc-N^{G4S-LLQGA}) and Trastuzumab-IgG (T-IgG) and Fab (T-Fab) reference constructs were designed. All proteins were expressed in Expi293F cells and purified by affinity chromatography (FIG. 6 and FIG. 7). Expression yields of Fcabs were reduced compared to huFc controls (mean yield: 54 mg/L versus 330 mg/L) (FIG. 8). C-terminal tagged variants S5-C^{G4S-LLQGA} and S5-C^{(G4S)2-LLQGA} aggregated during a protein concentration step and were excluded from further experiments. All other variants showed high purity confirmed via gel electrophoresis (SDS-PAGE) (FIG. 9) and analytical size-exclusion chromatography (SE-HPLC) (FIG. 10). The identity of all variants was confirmed by mass spectrometry (LC-MS) (FIG. 12). Variants were further functionalized via pHAb-dye or MMAE as described in the following sections.

TABLE 1
Fcabs and controls used in this study

| construct name | [symbol] | [symbol] | [symbol] | [symbol] | [symbol] |
|---|---|---|---|---|---|
| [symbol] | STAB5 Fcab | [symbol] A | — | 56.7 | — |
| [symbol] | STAB5 Fcab | [symbol] C | — | 56.8 | — |
| [symbol] | STAB5 Fcab | [symbol] A | [symbol] | [symbol] | — |
| [symbol] | STAB5 Fcab | [symbol] A | [symbol] | [symbol] | — |
| [symbol] | STAB5 Fcab | [symbol] A | [symbol] | [symbol] | — |
| [symbol] | STAB5 Fcab | [symbol] A | [symbol] | [symbol] | — |
| [symbol] | STAB [symbol] | [symbol] A | — | [symbol] | — |
| [symbol] | STAB [symbol] | [symbol] C | — | [symbol] | — |
| FS | [symbol] | [symbol] A | — | [symbol] | — |
| [symbol] | human [symbol] | [symbol] A | — | [symbol] | negative control |
| [symbol] -C265 | human [symbol] | [symbol] C | — | [symbol] | negative control |
| [symbol] | human [symbol] | [symbol] A | [symbol] | [symbol] | negative control |
| [symbol] | human [symbol] | [symbol] A | [symbol] | [symbol] | negative control |
| T-Fab | [symbol] | [symbol] | — | [symbol] | reference construct |
| T-IgG | [symbol] | — | — | [symbol] | reference construct |
| [symbol] | [symbol] | [symbol] A | — | [symbol] | 150 [symbol] |

Protein scaffold variants were modified for site-specific conjugation strategy (terminal LLQGA tags [symbol] or D265C [symbol] ) and effector function attenuation (D265A [symbol] ). Trastuzumab-Fab sequence was modified by K183C and V20SC THIOMAB positions. [symbol] EU numbering is used to specify amino acid positions. The exact size of each variant was determined [symbol] LC-MS and includes most intense glycosylation pattern. Amino acid sequences of the constructs are given in the supporting information (S [symbol] ). [symbol]
indicates data missing or illegible when filed

### Example 2: Conjugation of Fcabs and Control Antibodies with pHAb-Dye

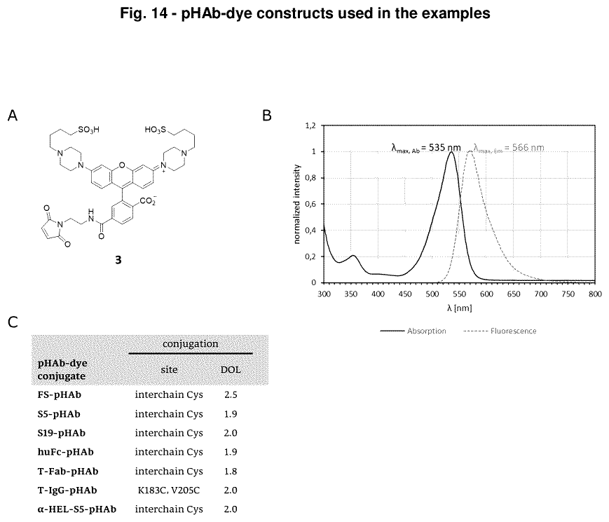

Selected Fcab variants and controls were labeled with a pH sensor fluorescent dye (pHAb-dye³⁵) via site-specific coupling to interchain cysteines (S5-pHAb, S19-pHAb, FS-pHAb, huFc-pHAb, T-IgG-pHAb, α-HEL-S5-pHAb) or engineered cysteines (T-Fab-pHAb) to study their cellular uptake and spheroid penetration profile. pHAb-dye is not fluorescent at neutral pH but becomes highly fluorescent at acidic pH present in endosomal and lysosomal vesicles after internalization.³⁵ Generated pHAb-dye conjugates had a defined degree of labeling (DOL) ranging between 1.8-2.5 as judged by UV-VIS spectroscopy. A detailed overview of pHAb-dye labeled constructs is given in FIG. 14.

### Example 3: Cellular Uptake of pHAb-Dye-Conjugates into Tumor Cells

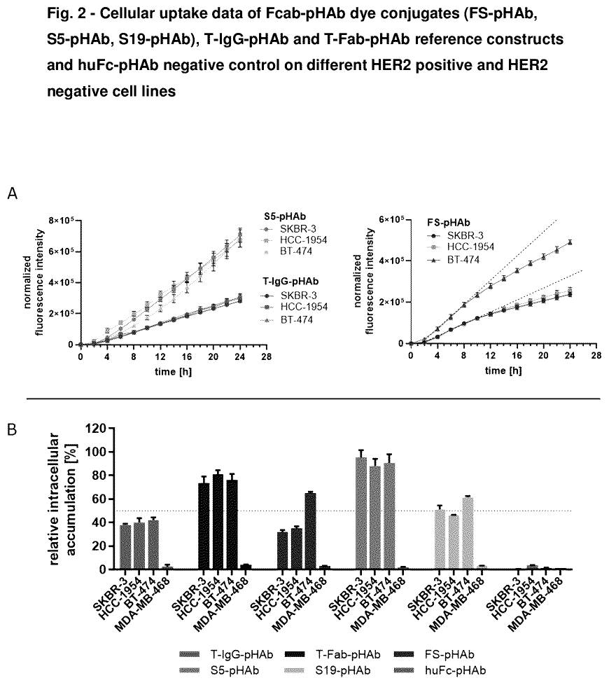

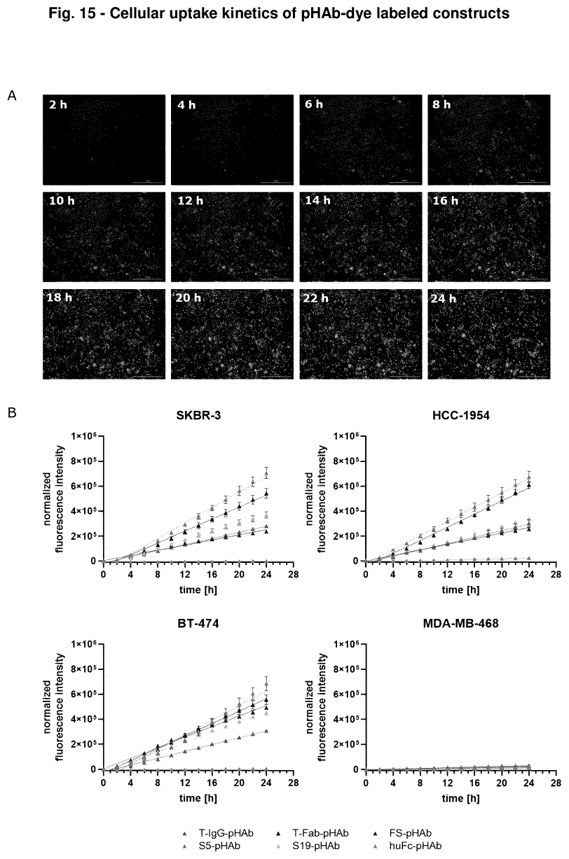

It was previously described that STAB19, STAB5 and FS102 bind to different HER2 epitopes than Trastuzumab.^{23,24} As this may impact internalization, lysosomal trafficking and ADC cytotoxicity of selected Fcabs, we investigated the cellular uptake profiles of pH-sensitive pHAb-dye conjugates on HER2 positive BT-474, SKBR-3, HCC-1954 and on HER2 negative MDA-MB-468 tumor cells. T-IgG-pHAb and T-Fab-pHAb were included in these experiments along with huFc-pHAb as a negative control. pHAb-dye labeled constructs were incubated on adherent cells for 24 h and cellular uptake kinetics were derived from increasing pHAb-dye fluorescence of cell images recorded every 2 h (FIG. 15A). Subsequently, the fluorescence intensity was normalized to cell numbers and to pHAb-dye DOL values of each construct (FIG. 2A) and linearly fitted (FIG. 15B).

The resulting normalized intracellular accumulation rates were then expressed relative to the highest rate (S5-pHAb on SKBR-3) (FIG. 2B). All Fcab-pHAb dye conjugates showed selective intracellular accumulation indicating internalization and endosomal trafficking thereby meeting the prerequisite for an ADC approach. Appreciable intracellular accumulation was most pronounced for S5-pHAb (K_{D}=2.25 nM), followed by T-Fab-pHAb (K_{D}=0.12 nM), S19-pHAb (K_{D}=46.6 nM), T-IgG-pHAb (K_{D}=0.18 nM) and FS-pHAb (K_{D}=0.34 nM). Reduced intracellular accumulation of S19-pHAb compared to S5-pHAb reflects reduced target engagement at subsaturating antibody concentrations used in this assay (100 nM), indicating a correlation between high HER2 binding affinity and elevated cellular uptake. Counterintuitively, variant FS-pHAb showed reduced intracellular accumulation although high affinity in receptor binding has been described. This can be attributed to profound HER2 degradation caused by FS102 that was reported to lower the density of surface displayed HER2²⁴ which would then be absent for consecutive internalization cycles. The HER2 depletion is also supported by the time dependent reduction of the intracellular accumulation rate (FIG. 2A). Higher intracellular accumulation of S5-pHAb compared to T-Fab-pHAb may be epitope-driven or result from enhanced endosomal HER2 dissociation (k_{off, pH} 7.4 2.61·10⁻³ s⁻¹ versus 0.13. 10⁻³ s⁻¹) enabling S5-pHAb entry into lysosomes while receptor bound T-Fab-pHAb is recycled.^{36,37} High recycling rates of Trastuzumab in HER2 high expressing cells are also described in literature.³⁸ Differences between T-IgG-pHAb and T-Fab-pHAb or S5-pHAb could be due to reduced receptor occupancy with fluorophore label considering that two receptors can be bound either by two labeled T-Fabs, Fcabs or one bivalent T-IgG-pHAb. In line with this, relative intracellular accumulation of T-IgG-pHAb was reduced by approximately 50% compared to T-Fab-pHAb or S5-pHAb. Lysosomal trafficking may also depend on the relative number of expressed surface receptors for which the following order has been reported SKBR-3>HCC-1954>BT-474.³⁸ In summary, these results demonstrate that HER2-Fcabs used in this study allow efficient intracellular accumulation required for ADC applications.

### Example 4: Generation of Fcab-Drug Conjugates

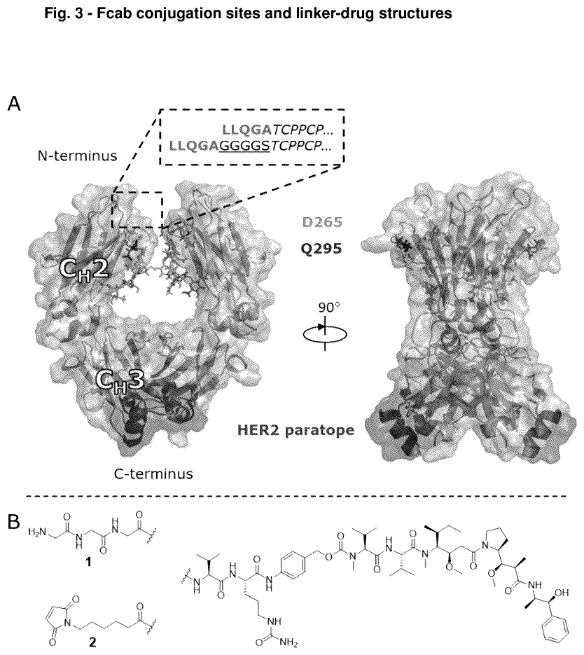

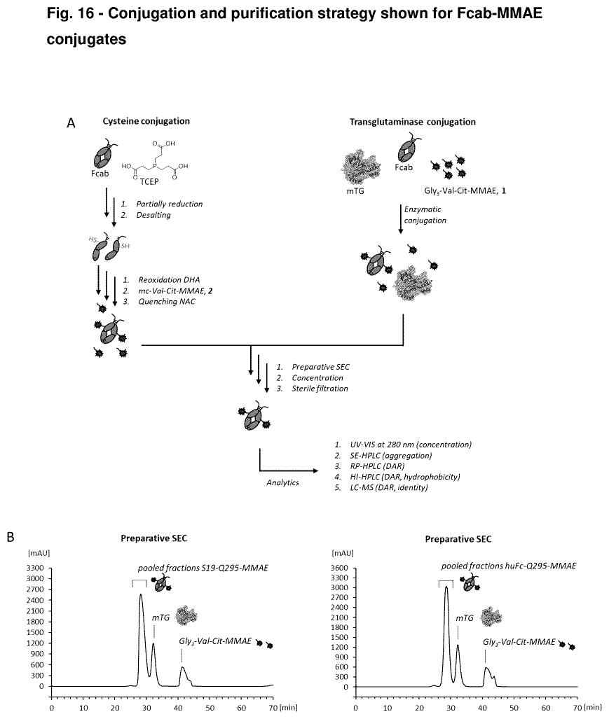

Several reports demonstrated the impact of conjugation sites on stability and therapeutic activity of ADCs.³⁹⁻⁴¹ Therefore, different sites and conjugation techniques were evaluated for the conjugation of Fcabs to linker-drugs (Table 2, FIG. 3A and FIG. 16). For this, the well-established cleavable valine-citrulline linker (Val-Cit) microtubule inhibitor MMAE construct with a glycine (Gly₃) handle (1, FIG. 3B) was conjugated via microbial transglutaminase (mTG) either to a genetically fused LLQGA tag at the N-terminus or to native Q295⁴² in the C_{H}2 domain (Table 2). In addition, cysteine conjugation to position D265C²⁸ was performed with Val-Cit-MMAE carrying a maleimidocaproyl (mc) handle (2, FIG. 3B) (Table 2).

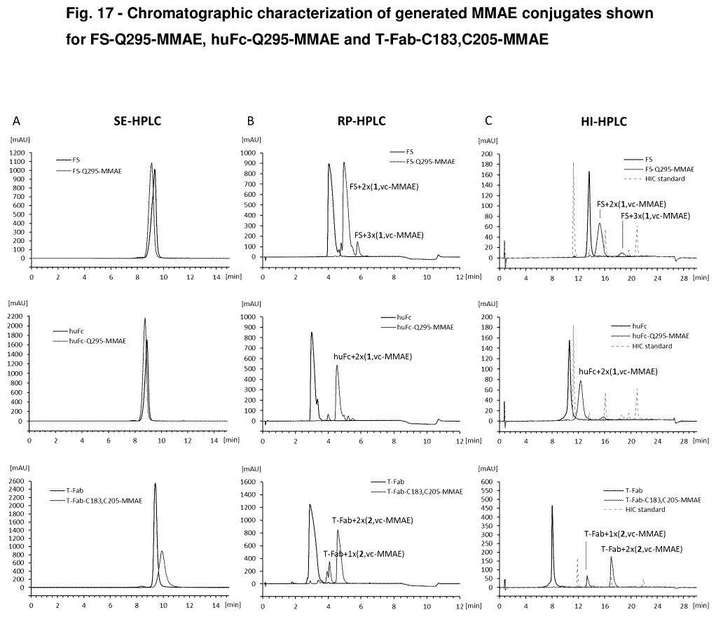

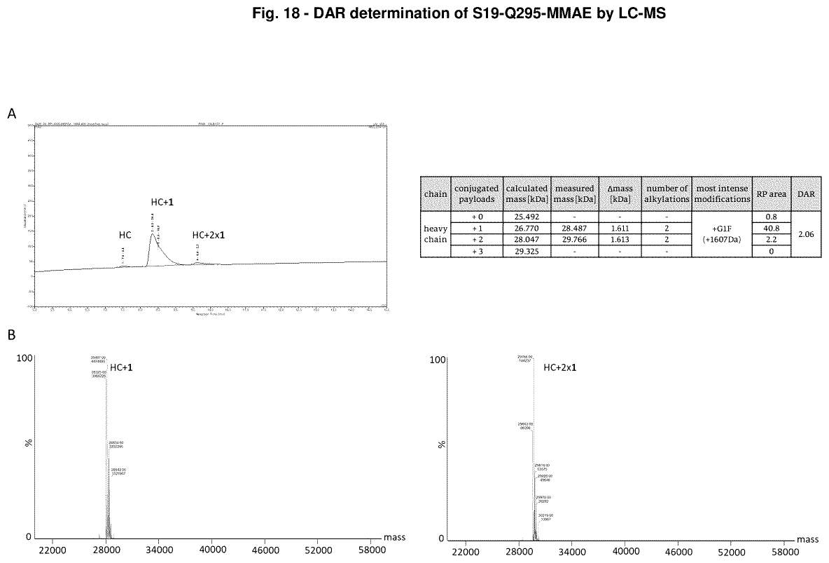

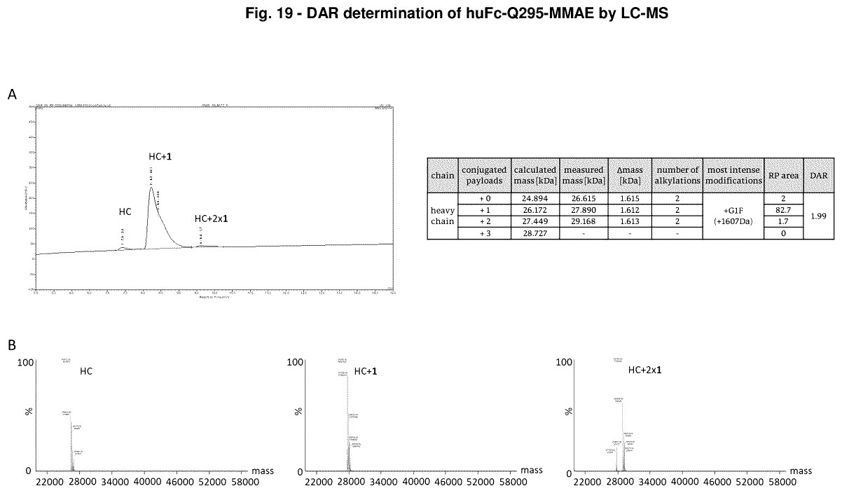

The absence of aggregates was confirmed by analytical SE-HPLC (Table 2 and FIG. 17A) and the drug-to-antibody ratio (DAR) was determined from reversed phase (RP-HPLC, FIG. 17B) and hydrophobic interaction chromatography (HI-HPLC, FIG. 17C) as well as LC-MS data (FIG. 18 and FIG. 19) (Table 2). Conjugation of 1 on N-terminal linked LLQGA tags was achieved by applying wild type mTG from S. mobaraensis which is reported to not recognize native Q295 in the IgG scaffold when N297 is glycosylated.⁴³ Surprisingly, the Fcab scaffold showed elevated DARs beyond DAR 2.0 (S5-N^{LLQGA}-MMAE DAR 2.4, S5-N^{G4S-LLQGA}-MMAE DAR 3.0) (Table 2) indicating that an additional glutamine residue was coupled via S. mobaraensis mTG.

No efforts were made to identify this position. For conjugation of 1 to native Q295 in the presence of glycosylated N297, we used a genetically engineered mTG as recently described⁴² and obtained homogeneous products with DAR 2.0-2.2 (S5-Q295-MMAE, S19-Q295-MMAE, FS-Q295-MMAE and huFc-Q295-MMAE) (Table 2). Cysteine conjugation at position D265C was less efficient for Fcabs (S5-C265-MMAE DAR 1.5, S19-C265-MMAE DAR 1.1) compared to an unmodified huFc control (DAR 1.8). Conjugation of hydrophobic payloads such as MMAE typically increases the overall hydrophobicity of the molecule. This can impact construct stability by protein aggregation and accelerate undesired non-specific uptake by normal cells.³² HI-HPLC was performed to estimate overall hydrophobicity from retention times (t_{R}) of DAR 2.0 drug conjugate peaks and unconjugated parent molecules (Table 2). Parent Fcab molecules showed higher hydrophobicity (t_{R} 13.22-16.49 min) compared to parent huFc (t_{R} 10.35-10.63 min). Accordingly, the overall hydrophobicity of Fcab-drug conjugates was elevated as well (t_{R} 14.39-19.55 min versus huFc conjugates t_{R} 12.33-18.05 min).

Moreover, the HI-HPLC relative retention time (RRT) can be calculated to characterize the shielding of hydrophobic payloads.^{42,44} Similar RRTs were measured for Q295 and C265 coupled Fcab-drug conjugates (RTT 1.04-1.12) indicating that MMAE is sufficiently shielded in these constructs (Table 2). huFc and S5 conjugate t_{R} and RRT increase for positions Q295<D265C<N-terminal LLQGA<N-terminal G₄S-LLQGA suggesting that position Q295 provides most efficient shielding and overall most reduced hydrophobicity. Along with superior conjugation yield and product homogeneity (DAR 2.0-2.2), position Q295 seems favorable for the generation of Fcab-drug conjugates.

TABLE 2
Generation of drug conjugates

| | | | | [symbol] -HPLC | | | |
|---|---|---|---|---|---|---|---|
| | | | | parental | DAR 2.0 | | SE-HPLC |
| | conjugation | | | construct | conjugate | | purity |
| drug conjugate | site | technique | DAR | [symbol] (min) | [symbol] (min) | RRT | [%] |
| SS- [symbol] -MMAE | native Q295 | [symbol] | 2.0 | 15.02 | [symbol] | 1.04 | 100.0 |
| SS- [symbol] -MMAE | D [symbol] | cysteine | [symbol] | [symbol] | [symbol] | 1.04 | 100.0 |
| SS- [symbol] -MMAE | N-term, LLQGA | [symbol] | 2.4 | 16. [symbol] | [symbol] | 1.19 | 100.0 |
| SS- [symbol] -MMAE | N-term, [symbol] -LLQGA | [symbol] | 3.0 | 16.49 | [symbol] | 1.18 | 100.0 |
| [symbol] -MMAE | [symbol] | [symbol] | 2.1 | [symbol] | [symbol] | 1.09 | 100.0 |
| [symbol] -MMAE | [symbol] | cysteine | [symbol] | [symbol] | [symbol] | 1.11 | 100.0 |
| [symbol] -MMAE | native [symbol] | [symbol] | 2.0 | 10. [symbol] | [symbol] | [symbol] | 99.8 |
| [symbol] -MMAE | [symbol] | [symbol] | 1.8 | 10. [symbol] | [symbol] | 1.16 | 100.0 |
| [symbol] -MMAE | [symbol] | cysteine | 1.4 | 10. [symbol] | [symbol] | 1.27 | 100.0 |
| [symbol] -MMAE | N-term LLQGA | [symbol] | [symbol] | 10. [symbol] | [symbol] | [symbol] | 100.0 |
| [symbol] -MMAE | N-term [symbol] | [symbol] | [symbol] | 10.43 | 18.05 | [symbol] | 100.0 |
| [symbol] -MMAE | [symbol] | cysteine | [symbol] | 8.01 | 16.99 | 2.12 | 99.6 |
| [symbol] -MMAE | native [symbol] | [symbol] | 2.0 | 10.92 | [symbol] | 1.20 | 100.0 |

DAR is given as a means from H [symbol] -HPLC, RP-HPLC and LC-MS analysis. RRT was calculated from the [symbol] of the DAR 2.0 drug conjugate and the parental construct as hydrophobicity measure added by MMAE. SE-HPLC purity refers to the final drug conjugate and was analyzed after a freeze-thaw cycle. [symbol]
indicates data missing or illegible when filed

### Example 5: Receptor Binding Properties of Drug Conjugates

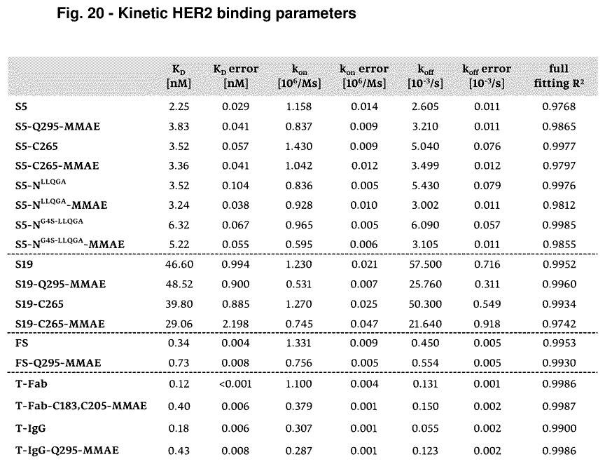

To evaluate whether the conjugation of hydrophobic payloads such as MMAE alters the binding behavior of Fcab-drug conjugates to their target HER2 or FcRn receptor, dissociation constants (K_{D}) of Fcab- and control conjugates to recombinant HER2 or FcRn were determined via biolayer interferometry (BLI) and compared to their unconjugated parent variants (Table 3, FIG. 20-23).⁴⁵ For both, 10 HER2 as well as FcRn, dissociation constants were not affected by conjugation.

TABLE 3
HER2 and FcRn binding affinity of unconjugated and
conjugated Fcabs and Trastuzumab-based controls

| | unconjugated parent | | MMAE-conjugates | |
|---|---|---|---|---|
| | K_{D} (HER2) | K_{D} (FcRn) | K_{D} (HER2) | K_{D} (FcRn) |
| drug conjugate | [nM] | [nM] | [nM] | [nM] |
| S5-Q295-MMAE | 2.25 ± 0.03 | 399 ± 15 | 3.83 ± 0.04 | 274 ± 11 |
| S5-C265-MMAE | 3.52 ± 0.06 | 376 ± 13 | 3.36 ± 0.04 | 350 ± 35 |
| S5-N [symbol] -MMAE | 3.52 ± 0.10 | 389 ± 13 | 3.24 ± 0.04 | 284 ± 9 |
| S5-N [symbol] -MMAE | 6.32 ± 0.07 | 357 ± 12 | 5.22 ± 0.06 | 226 ± 8 |
| S19-Q295-MMAE | 46.6 ± 0.99 | 383 ± 13 | 48.5 ± 0.99 | 609 ± 25 |
| S19-C265-MMAE | 39.8 ± 0.89 | 448 ± 15 | 29.1 ± 2.20 | 305 ± 36 |
| FS-Q295-MMAE | 0.34 ± 0.004 | 346 ± 11 | 0.73 ± 0.008 | 339 ± 11 |
| huFc-Q295 | — | 524 ± 13 | — | 501 ± 12 |
| T-Fab-C183, C205-MMAE | 0.12 ± 0.001 | n.d. | 0.40 ± 0.006 | n.d. |
| T-IgG-Q295-MMAE | 0.18 ± 0.006 | 303 ± 9 | 0.43 ± 0.008 | 387 ± 14 |

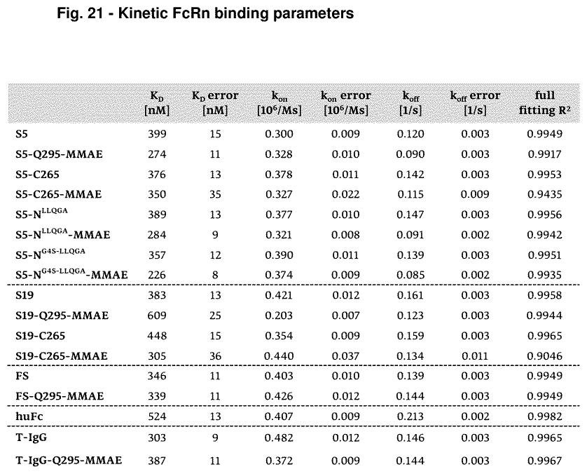

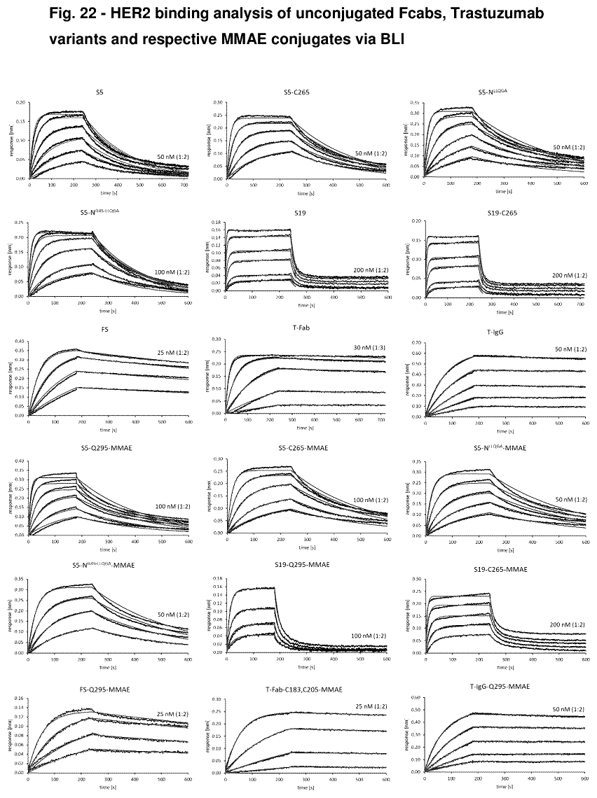

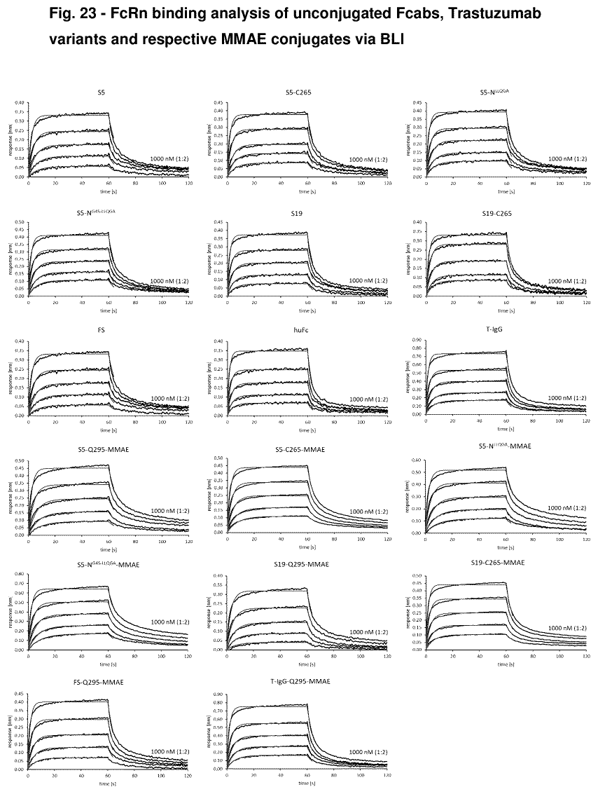

Dissociation constants (K_{D}) were measured by BLI using recombinantly produced HER2 or FcRn. Binding affinity to FcRn was determined at pH 6.0. Errors are standard errors from fitting using FortéBio data analysis software 9.1. On- and off-rates as well as curve fittings are included in the supporting information (FIG. 20, FIG. 21, FIG. 22 and FIG. 23). (n.d., not determined) [symbol]
indicates data missing or illegible when filed

### Example 6: Serum Stability of Drug Conjugates

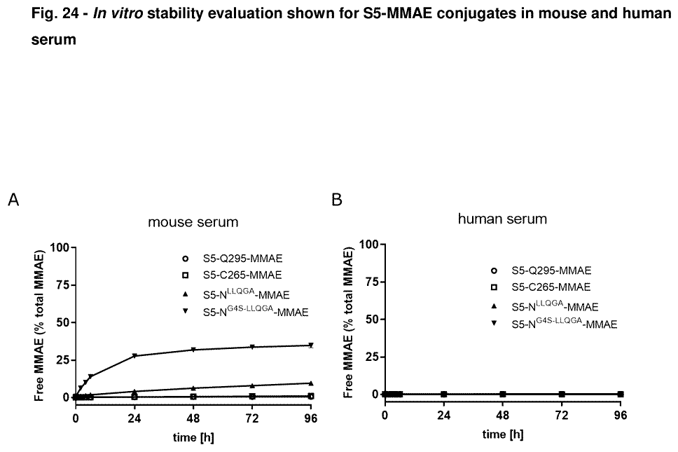

Pharmacokinetics of drug conjugates not only depend on FcRn binding but are also impacted by conjugate stability for that a pronounced conjugation site dependency has been documented.³⁹⁻⁴¹ To evaluate drug-conjugate stability in serum, we incubated the Fcab-drug conjugates along with Trastuzumab-based drug conjugates in mouse and human serum and monitored payload release by detection of free MMAE via LC-MS/MS (Table 4, FIG. 24). No free MMAE was measured for all conjugates in human serum. Similar high stability was measured in mouse serum for variants carrying Val-Cit-MMAE on position Q295 or D265C, which is in-line with previously reports that Q295 conjugation site confers great stability to full-length ADCs.^{41,42,46} Interestingly, S5-N^{LLQGA}-MMAE (9.6%) and S5-N^{G4S-LLQGA}-MMAE (34.8%) showed elevated MMAE release, likely due to the solvent exposed position at the N-terminus favoring serum protease accessibility. Herein, it is well described that Val-Cit linkers can undergo cleavage in mouse serum mediated by a murine extracellular carboxylesterase 1c (mCes1c)⁴⁷ and that either conjugation site⁴¹ or linker design^{47,48} could prevent cleavage. Elevated MMAE release and higher solvent exposure may also be reflected by higher HI-HPLC RRT of N-terminal linked MMAE constructs (Table 2).⁴⁴

TABLE 4
Serum stability of Fcab- and Trastuzumab-based drug conjugates

free MMAE
(% total MMAE)

| drug conjugate | mouse | human |
|---|---|---|
| S5-Q295-MMAE | 0.5 | 0.2 |
| S5-C265-MMAE | 1.0 | 0.0 |
| S5-N [symbol] -MMAE | 9.6 | 0.2 |
| S5-N [symbol] -MMAE | 34.8 | 0.0 |
| S19-Q295-MMAE | 0.6 | 0.3 |
| S19-C265-MMAE | 1.4 | 0.0 |
| FS-Q295-MMAE | 0.4 | 0.0 |
| T-Fab-C183, C205-MMAE | 1.6 | 0.0 |
| T-IgG-Q295-MMAE | 0.5 | 0.0 |

Free MMAE was measured via LC MS/MS after incubation in mouse and human sera at 37° C. for 96 h (n = 3). Numbers show the released fraction relative to initially conjugated MMAE. [symbol]
indicates data missing or illegible when filed

### Example 7: In Vitro Cytotoxicity

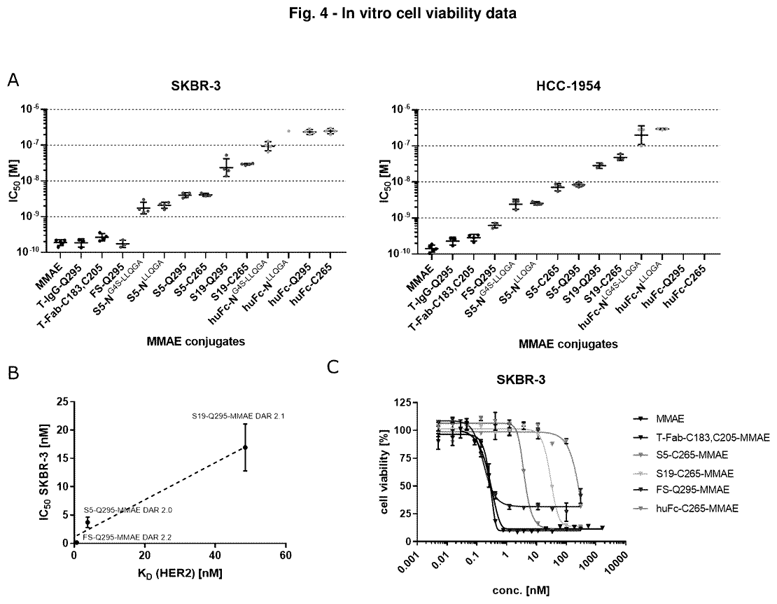

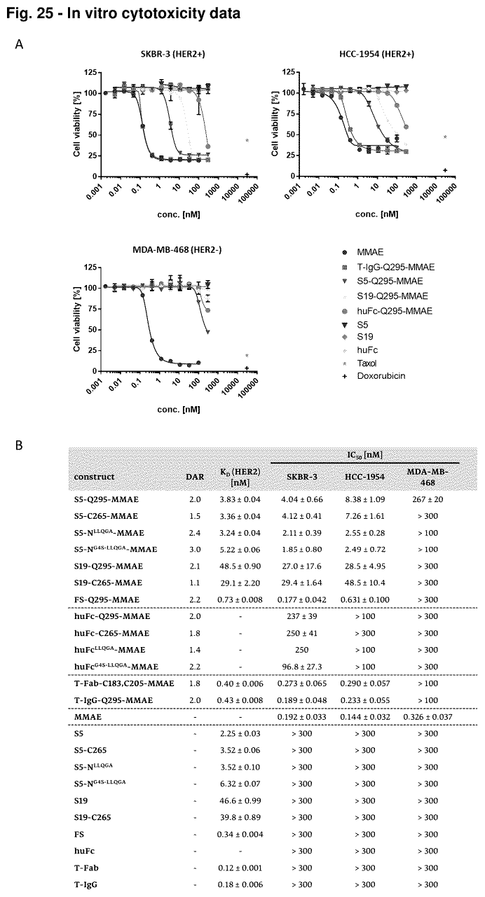

To examine whether the generated Fcab-drug conjugates selectively deliver and efficiently release MMAE in cells, MMAE conjugates were incubated on HER2 overexpressing (SKBR-3, HCC-1954) and HER2 negative (MDA-MB-468) cell lines (FIG. 4 and FIG. 25). The Fcab-drug conjugates (DAR 1.1-3.0) were evaluated along with T-IgG-Q295-MMAE (DAR 2.0) and T-Fab-C183, C205-MMAE (DAR 1.8) reference conjugates and huFc-MMAE (DAR 1.4-2.2) as well as unconjugated Fcab negative controls. All Fcab-drug conjugates and Trastuzumab-based control conjugates demonstrated selective cytotoxicity on HER2 positive cells with IC₅₀ values ranging from subnanomolar to double digit nanomolar concentrations (FIG. 4A), whereas greatly reduced cytotoxicity (IC₅₀>100 nM) was measured on HER2 negative cells (FIG. 25). By contrast, huFc-MMAE negative controls showed only little cytotoxic effects at higher concentrations (IC₅₀>100 nM), and unconjugated Fcabs did not mediate any cytotoxic effects on SKBR-3, HCC-1954 or MDA-MB-468 cells (FIG. 25). S5 and S19-based MMAE conjugates showed 10 to 100-fold reduced potency, compared to T-IgG and T-Fab MMAE conjugates, that correlates with lower HER2 affinities (Table 3 and FIG. 4B). FS-Q295-MMAE shows high potency (IC₅₀ 0.18 nM) but lower reduction of cell viability (78% versus 87-95% for other constructs) on SKBR-3 cells which may be caused by its reported HER2 receptor degradation preventing cells from being exposed to a cytotoxic dose of payload (FIG. 4C). Overall, these results demonstrate that Fcab-drug conjugates promise to be safe and efficacious due to selective cell killing and that tuning the affinity heavily impacts in vitro cytotoxicity.

### Example 8: 3D Tumor Spheroid Penetration Studies Using pHAb-Dye-Conjugates

To estimate efficacy in animal models, in vitro cytotoxicity data can be misleading as additional effects need to be considered. For example, Nessler et al. evaluated for various single-domain antibody-drug conjugates the impact of target receptor affinity on in vitro potency, biodistribution and in vivo efficacy for a solid tumor xenograft model.¹⁶ Constructs with subnanomolar receptor affinity and lower in vitro potency counterintuitively showed higher in vivo efficacy. Biodistribution profiles indicated that lower affinity of variants increased the tumor penetration and in vivo activity.¹⁶ Therefore, it is tempting to speculate that Fcab-drug conjugates may show elevated solid tumor penetration, compared to higher affinity full-size ADC variants.

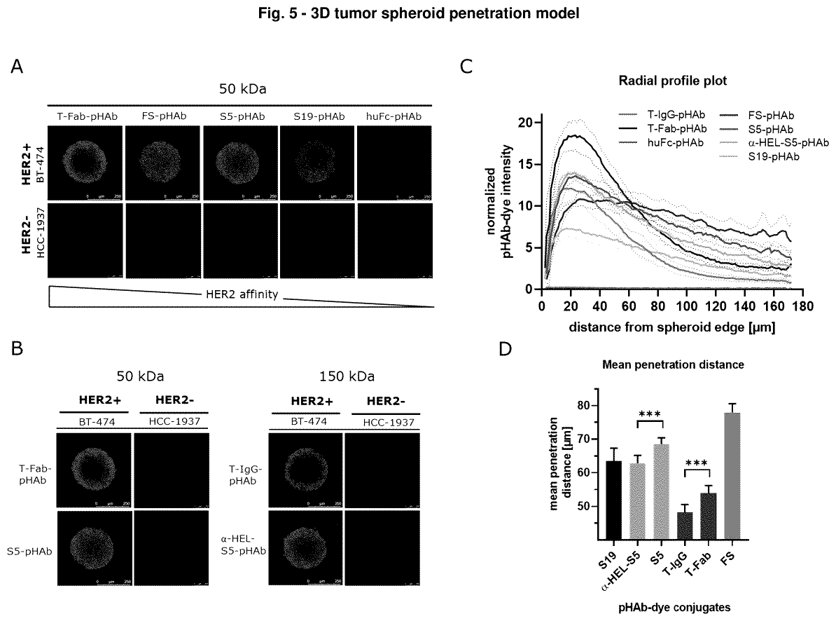

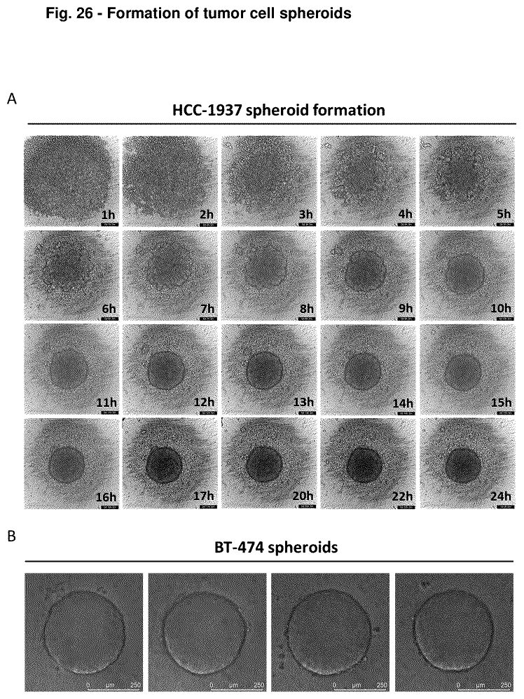

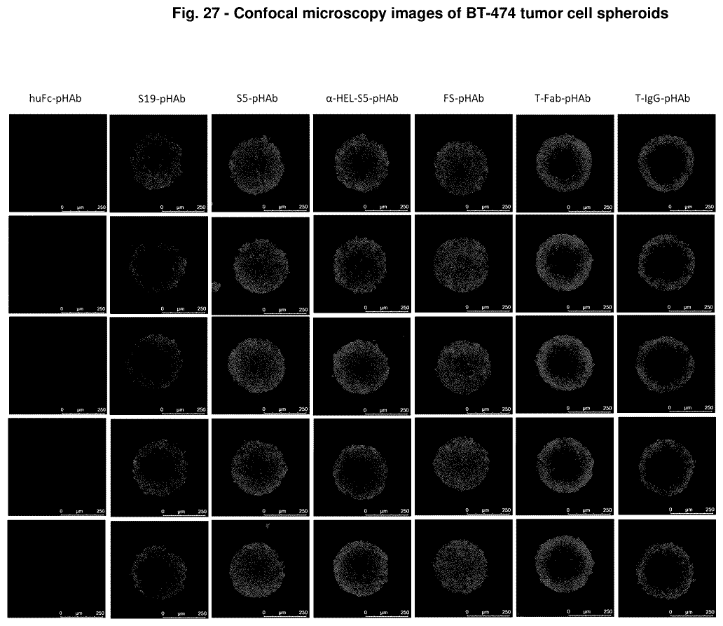

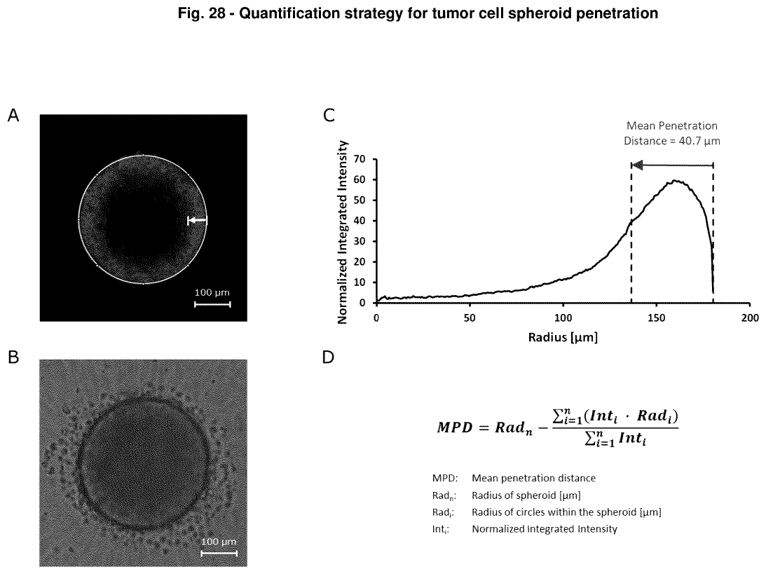

To anticipate tumor penetration in vitro, we established a cellular tumor spheroid penetration model. For this, cell screenings were performed to identify HER2-positive BT-474 and HER2 negative HCC-1937 cell lines that form round spheroids at reproducible size (FIG. 26). For penetration experiments confocal microscopy was applied together with pHAb-dye conjugates (FS-pHAb, S5-pHAb, S19-pHAb, huFc-pHAb, T-IgG-pHAb, T-Fab-pHAb, α-HEL-S5-pHAb) due to their favorable signal over background ratio.³⁵ To quantify spheroid penetration, a novel analysis strategy was applied that allowed for the calculation of the mean penetration distance (MPD) from radial profile plots of confocal microscopy images (details can be found in the material and methods section, FIG. 27 and FIG. 28). To study the impact of target affinity on distribution and cellular uptake, T-Fab-pHAb, FS-pHAb, S5-pHAb, S19-pHAb and huFc-pHAb were incubated on HER2 overexpressing BT-474 spheroids and the distribution of intracellular accumulated constructs was analyzed by fluorescence measurements via confocal microscopy (FIG. 5A). High affinity T-Fab-pHAb (K_{D} 0.12 nM) accumulated in the periphery of the tumor spheroid (MPD 54±2 μm). This restricted accumulation is probably caused by extensive binding and internalization which oppose transport towards the center of the spheroid and prevent further penetration—an observation described as “binding site-barrier” in the literature.^{18,49} In line with this, lower affinity variants S5-pHAb (K_{D} 2.25 nM) and S19-pHAb (K_{D} 46.60 nM) showed a more homogenous distribution and elevated MPD (69±2 μm and 63±4 μm) compared to T-Fab-pHAb. In contrast, FS-pHAb showed the most homogeneous distribution and highest MPD (78±3 μm) despite its high affinity (K_{D}=0.34 nM). Receptor degradation mediated by FS-pHAb may lead to reduction of endocytotic clearance (lower intracellular accumulation signal, FIG. 5C) thereby improving spheroid penetration. Beside FS-pHAb, S5-pHAb showed the highest MPD (69±2 μm), indicating that in these assays a single-digit nanomolar binding affinity seems beneficial for pronounced intracellular accumulation and spheroid penetration. Importantly, no pHAb-dye conjugate showed any signal on HER2 negative HCC-1937 spheroids. huFc-pHAb showed also no signal on BT-474 spheroids. Beside target binding affinity, the hydrodynamic radius impacts tumor spheroid penetration. Therefore, the penetration profile of 50 kDa Fcab molecule S5-pHAb was compared to its 150 kDa derivative a-HEL-S5-pHAb along with T-Fab-pHAb and T-IgG-pHAb controls (FIGS. 5B and 5C). As expected, smaller-sized S5-pHAb penetrated deeper into BT-474 spheroids (MPD 69±2 μm) compared to α-HEL-S5-pHAb (MPD 63±2 μm) (FIG. 5D). The bivalent 150 kDa T-IgG-pHAb reference conjugate (MPD 48±2 μm) showed a binding site-barrier effect that was more pronounced compared to monovalent 50 kDa T-Fab-pHAb (MPD 54±2 μm) suggesting elevated affinity in binding to cellular HER2 due to avidity effects. Taken together, improved penetration capability of S5-pHAb, S19-pHAb and FS-pHAb compared to T-Fab-pHAb and T-IgG-pHAb was demonstrated resulting from fine-tuned lower affinity, smaller size and an intrinsic receptor degradation mechanism. Whether this effect translates in better efficacy in vivo needs to be investigated in carefully designed animal models considering additional effects such as plasma clearance and tumor tissue extravasation.

### Example 9: Material and Methods

### Plasmid Generation

Amino acid sequences of antibody fragments were taken from literature (STAB527, STAB19²⁷, FS102²⁴, huFc²³, Trastuzumab-Fab50) and modified as stated in table 1. For clarity, amino acid sequences are also given in the SI. pTT5 plasmids containing the modified sequences were ordered from GeneArt (Thermo Fisher Scientific) as codon-optimized versions for mammalian expression.

### Preparation of Antibody Fragments

Fcabs and huFc controls were expressed by transient transfection of heavy chains (+light chain in the case of T-Fab) in Expi293F™ cells following the manufacturer's instructions using the corresponding transfection kit and media from Life Technologies. Supernatant was harvested after 5 days post transfection. T-Fab contained a His₆-Tag for purification and was dialyzed against phosphate-buffered saline (PBS) pH 7.4 overnight before immobilized metal affinity chromatography (1 mL HisTrap™ HP, GE Healthcare) using an ÄKTA Pure device (GE Healthcare). Fcabs and huFc controls were purified by protein A affinity chromatography using HiTrap™ Mab Select SuRe 5 mL columns (GE Healthcare) and subsequently formulated in PBS pH 6.8 using HiPrep™ 26/10 desalting columns. Antibody purity was analyzed by analytical SE-HPLC using a TSKgel® SuperSW3000 column (Tosoh Bioscience) and by SDS gel electrophoresis. Identity of proteins was confirmed via intact mass analysis by LC-MS using a TripleTOF® 6600+ mass spectrometer (AB Sciex). Antibody-fragments were concentrated using Ultra centrifugal filter units (3K MWCO, Amicon®), sterile filtered and protein concentration was determined by UV-VIS spectroscopy at 280 nm. Antibody-fragments were snap-frozen in liquid nitrogen and stored at −80° C.

Preparation of pHAb-Dye Conjugates

For thiol coupling, antibodies and antibody fragments were reduced with 2.5 mM DTT in DPBS, 1 mM EDTA, pH 7.0 for 1.5 h at 25° C., 450 rpm. DTT was removed by Zeba™ Spin desalting columns equilibrated with DPBS, 1 mM EDTA, pH 7.0. 2.0 molar equivalents (pHAb: antibody) of pHAb thiol reactive dye (10 mg/mL 1:1 (v/v) DMSO/H₂O, Promega) were added to the reduced antibodies and antibody fragments and incubated for 3 h at 25° C., 450 rpm in the absence of light. No unreacted pHAb-dye was left and DOL values could be determined by UV-VIS spectroscopy according to the manufacturer's instructions.

### Preparation of MMAE Conjugates

Transglutaminase conjugation: mTG-mediated antibody conjugation was assessed in reactions with 5 mg/mL antibody or antibody-fragments, 20 equivalents of drug-linker and 60 U/mL genetically engineered mTG (made in-house⁴²) for conjugation on Q295 or 6 U/mL mTG from S. mobaraensis (Zedira) for conjugation on LLQGA tags in PBS pH 6.8 with up to 10% DMSO. Activity of mTG (U/mL) was determined using the ZediXclusive microbial transglutaminase (Zedira) photometric assay. Antibody fragments were used as prepared, Trastuzumab was purchased from pharmacy (Herceptin) and drug-linker Gly₃-Val-Cit-PAB-MMAE (1) was purchased from Levena. Reaction mixes were incubated at 37° C. for 18 h with gentle shaking, chilled to 10° C. and purified by preparative size exclusion chromatography (SEC) (Figure S10).

Cysteine conjugation: Antibody fragments were diluted to a final concentration of 5 mg/mL in PBS pH 7.4, 1 mM EDTA and partially reduced with an excess of 40 equivalents tris(2-carboxyethyl) phosphine (TCEP) for 2 h at 37° C. TCEP was removed via two consecutive 5 mL HiTrap™ Desalting Columns (GE Healthcare) and the reduced antibody fragments were reoxidized with 20 equivalents dehydroascorbic acid for 2 h at 25° C. To this mixture, 8 equivalents of mc-Val-Cit-PAB-MMAE (2) (Levena) were added and incubated for 1 h at 25° C. before the reaction was stopped by the addition of 25 equivalents of N-acetylcysteine (15 min at 25° C.) and purified by preparative SEC.

Preparative SEC was performed using either a Superdex™ 200 Increase 10/300 GL, Superdex™ 75 10/30 GL or a Superdex™ 200 prep grade 16/60 column in a 1260 liquid chromatography system (Agilent Technologies) or an ÄKTA Avant device (GE Healthcare) with PBS pH 6.8 as running buffer. Purified conjugates were concentrated using Ultra centrifugal filter units (10K MWCO, Amicon®), sterile filtered and protein concentration was determined by UV-VIS spectroscopy at 280 nm. The purified conjugates were subjected to analysis by SE-HPLC and DAR determination (HIC, RP, LC-MS) as described elsewhere⁴², snap-frozen in liquid nitrogen and stored at −80° C.

### Cell Culture

Human cancer cell lines were obtained from the American Type Culture Collection (HER2 positive: BT-474, HCC-1954, SKBR-3; HER2 negative: HCC-1937, MDA-MB-468) and maintained according to standard culture conditions (37° C., 5% CO₂, 95% humidity). SKBR-3 cells were cultured in DMEM high glucose medium supplemented with 10% fetal bovine serum (FBS), 2 mM L-glutamine and 1 mM sodium pyruvate. HCC-1954, HCC-1937 and MDA-MBA-468 were cultured in Roswell Park Memorial Institute (RPMI) 1640 medium supplemented with 10% FBS, 2 mM L-glutamine and 1 mM sodium pyruvate. BT-474 cells were cultured in Ham F12 medium supplemented with 10% fetal bovine serum (FBS), 2 mM L-glutamine, 1 mM sodium pyruvate and 10 μg/mL insulin. For subculturing, adherent grown cells were detached by adding 0.05% trypsin-EDTA, diluted with fresh medium and transferred into a new culturing flask.

### Cellular Uptake Assay

An appropriate number of cells was centrifuged at 500×g for 5 min. The supernatant was discarded, and cells were resuspended in the respective medium without phenol red at 200,000 vc/mL. The cell suspension (40 μL/well) was seeded into a black 384 clear bottom plate followed by incubation (37° C., 5% CO₂) in a humid chamber overnight. pHAb-dye constructs were supplemented with 0.3% Tween-20 (final), diluted to 3 UM and added in triplicates to the cells (final 100 nM) using a D300e digital dispenser (Tecan). The cells were immediately transferred to a Cytation 5 cell imaging reader (BioTek) equipped with DAPI and RFP filter cubes and a BioSpa 8 automated incubator (BioTek). Brightfield (objective: 10×, LED intensity: 10, integration time: 13 msec, camera gain: 24) and RFP channel images (ex.: 531 nm, em.: 593 nm, LED intensity: 10, integration time: 60 msec, camera gain: 24) were taken every 2 h over a period of 24 h. About 30 min before the 24 h measurement, the plate was removed from the BioSpa 8 device and 1 μg/mL Hoechst 33342 dye was added via a Tecan D300e digital dispenser for an additional 24 h endpoint DAPI nuclear staining image. Images were processed by the BioTek gen5 data analysis software. The total pHAb dye fluorescence intensity (RFP channel) of each image was normalized to the number of cells determined in the DAPI channel and subtracted by the RFP channel signal at 0 h (background signal). The cell number and background normalized intensities were divided by the pHAb-dye DOL value of each construct and plotted against the time. Data was fitted by linear regression in GraphPad Prism (GraphPad Software, Inc.) and intracellular accumulation rates (slopes) were derived. Finally, the relative intracellular accumulation (%) was calculated for each construct based on the highest intracellular accumulation rate.

FcRn and HER2 Binding Kinetic binding parameters were determined by BLI using the Octet® RED96 system (FortéBio, Pall) at 30° C. and 1,000 rpm agitation speed.

For HER2 binding analysis of Fcab variants, T-Fab and their conjugates (analytes), anti-mouse IgG Fc capture biosensors (AMC) were loaded with murine Fc-HER2 dimer (20 μg/mL diluted in DPBS, made in-house) for 360 s. Biosensors were then transferred into kinetics buffer (PBS pH 7.4, 0.02% Tween-20 and 0.1% bovine serum albumin) and incubated for 45 s followed by an association step to the analytes. Analytes were diluted in kinetics buffer in a concentration range varying from 200 nM to 3.13 nM. Association was monitored for 180 s or 240 s followed by a dissociation step in kinetics buffer for 480 s to determine kₒₙ and k_{off} values. Analytes were replaced by kinetics buffer, serving as a negative control and reference measurement. Respective non-binding human Fc fragments were used as negative controls in each experiment. The buffer reference measurement (control curve) was subtracted from antibody measurements for data fitting and kinetics parameter were determined by using FortéBio data analysis software 12.0 applying a 1:1 global full-fit binding model after Savitzky-Golay filtering. For HER2 binding analysis of T-IgG and its MMAE conjugate, a reversed assay set-up using monomeric HER2-His₆ (Novoprotein) as analyte was chosen to avoid avidity effects. After a 60 s baseline step in DPBS, antibodies (10 μg/mL in DPBS) were loaded for 60 s on anti-human IgG Fc capture biosensors (AHC) followed by a 45 s kinetics buffer step. Association of HER2-His₆ (50-0.78 nM) (diluted in kinetics buffer) was monitored for 180 s before a final dissociation step in kinetics buffer for 420 s. Buffer reference measurements were included and data was processed as mentioned before.

The FcRn binding assay was adapted from a published FortéBio application note. 45 Baseline, association and dissociation steps were performed in sodium phosphate buffer (100 mM sodium phosphate, 150 mM NaCl, 0.05% Tween-20, pH 6.0). The same buffer was used for dilution of analytes and ligand. Streptavidin biosensors were used and sensorgrams were recorded at 10 Hz starting with a 60 s baseline step before biotinylated FcRn-His₆ (made in-house) (2 μg/mL) was captured for 120 s. Subsequently, association of Fcabs, T-IgG and their respective MMAE conjugates was measured at varying concentrations (1 μM to 15.63 nM) for 60 s followed by dissociation for 60 s. A reference measurement with loaded biosensor omitting analyte association was included in each run to account for ligand dissociation. To subtract unspecific binding to the sensor tips, the assay was run again with unloaded reference biosensors. After subtracting the reference measurement and the reference sensor run (double referencing), a Savitzky-Golay filtering was performed and data was fitted using a 1:1 global partial-dissociation model. Due to the typical biphasic dissociation, the dissociation step was only fitted for 4 s to cover the initial fast dissociation rate.⁴⁵

### Serum Stability

The serum stability assay was conducted as previously described⁴² applying some minor modifications: MMAE conjugates were incubated at a final concentrations of 5 μM conjugated MMAE (considering the DAR of each construct) in human and mouse serum. Moreover, serum samples were supplemented with 5 μM deuterated D8-MMAE as internal standard.

### Cytotoxicity Assay

For the evaluation of Fcab-MMAE conjugates and related compounds, 40 μL of viable cell suspension were seeded into opaque 384 well plates (SKBR-3:6000 vc/well, HCC-1954:3500 vc/well, MDA-MB-468:2500 vc/well) followed by incubation (37° C., 5% CO₂) in a humid chamber overnight. Test compounds were added using a D300e digital dispenser (Tecan). Free MMAE and protein/protein-conjugate solutions were supplemented with 0.3% Tween-20 (final) and diluted to 6 μM (MMAE) or 10 μM (proteins). All wells were normalized to the maximum amount of Tween-20 added. Cell viability was determined after 4 d using Cell Titer Glo reagent (Promega) according to the manufacturer's instructions. Luminescence values were normalized to luminescence of non-treated cells and dose-response was fitted using the asymmetric (five parameter) fitting function of GraphPad Prism (GraphPad Software, Inc.).

### Spheroid Penetration Assay

For spheroid formation, BT-474 or HCC-1937 cells were diluted in their appropriate medium and seeded (2,000 vc/well; 40 μL) into a black clear/round bottom 384 well plate (Corning). The plate was centrifuged for 4 min at 660×g, rotated by 180° and centrifuged for further 4 min at 660×g to center the cells in the middle of the wells. Cells were incubated for 96 h at 37° C., 5% CO₂ in a humid chamber to allow formation of spheroids. pHAb-dye constructs were supplemented with 0.3% Tween-20 (final), diluted to 3 μM and added in replicates (n=8) to the cells (final 50 nM) using a D300e digital dispenser (Tecan). BT-474 and HCC-1937 spheroids were incubated for 24 h at 37° C., 5% CO₂ in a humid chamber, under exclusion of light. Images were taken with a Leica TCS SP8 Confocal Laser Scanning Microscope (20× objective, excitation: 535 nm, emission: 560-610 nm, laser power: 20, gain: 500). Radial profile plots were created from unprocessed images using the radial profile plot plug-in in ImageJ⁵¹ (Figure S20) and normalized to the pHAb-dye DOL value of each construct. Mean penetration distances were calculated from ImageJ data by the following equation, where rad is the radius of the spheroid in μm, rad; the radius of concentric circles within the spheroid in μm, and intᵢ the normalized integrated intensity on circle with radius radᵢ.

$$mean penetration distance = {rad}_{n} - \frac{{\sum }_{i = 1}^{n} ( {int}_{i} \cdot {rad}_{i} )}{{\sum }_{i = 1}^{n} {int}_{i}}$$

### Example 10: Injection Vials

A solution of 100 g of a conjugate of the present invention and 5 g of disodium hydrogenphosphate in 3 I of bidistilled water is adjusted to pH 6.5 using 2 N hydrochloric acid, filtered under sterile conditions, transferred into injection vials, lyophilised under sterile conditions and sealed under sterile conditions. Each injection vial contains 5 mg of a conjugate of the present invention.

### Example 11: Solution

A solution is prepared from 1 g of a conjugate of the present invention, 9.38 g of NaH₂PO₄ 2 H₂O, 28.48 g of Na₂HPO₄·12 H₂O and 0.1 g of benzalkonium chloride in 940 ml of bidistilled water. The pH is adjusted to 6.8, and the solution is made up to 1 I and sterilised by irradiation.

### Example 12: Ampoules

A solution of 1 kg of a conjugate of the present invention in 60 I of bidistilled water is filtered under sterile conditions, transferred into ampoules, lyophilised under sterile conditions and sealed under sterile conditions. Each ampoule contains 10 mg of a conjugate of the present invention.

### Example 13: Amino Acid Sequences of Expressed Proteins

### 1. Fcabs

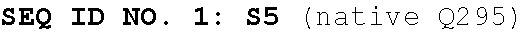

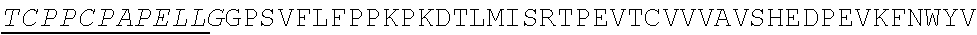

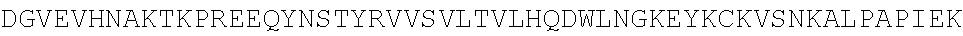

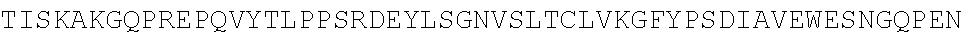

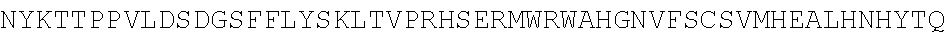

KSLSLSPG

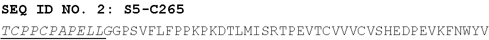

DGVEVHNAKTKPREEQYNSTYRVVSVLTVLHQDWLNGKEYKCKVSNKALPAPIEK

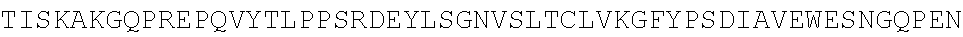

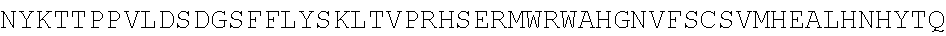

KSLSLSPG

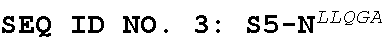

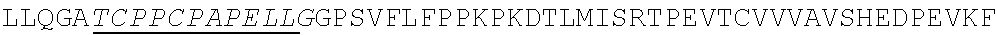

NWYVDGVEVHNAKTKPREEQYNSTYRVVSVLTVLHQDWLNGKEYKCKVSNKALP

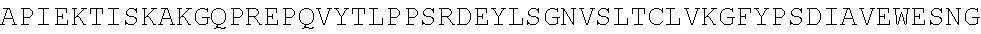

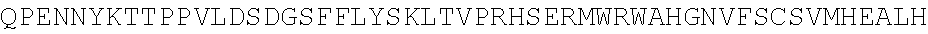

NHYTQKSLSLSPG

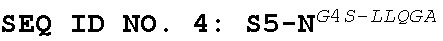

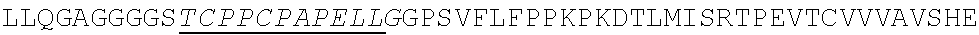

DPEVKFNWYVDGVEVHNAKTKPREEQYNSTYRVVSVLTVLHQDWLNGKEYKCKV

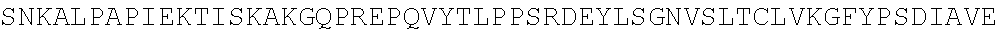

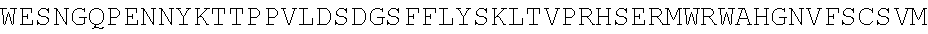

HEALHNHYTQKSLSLSPG

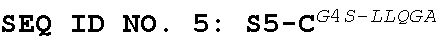

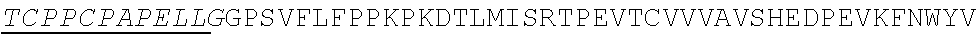

DGVEVHNAKTKPREEQYNSTYRVVSVLTVLHQDWLNGKEYKCKVSNKALPAPIEK

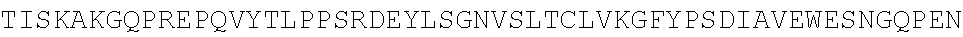

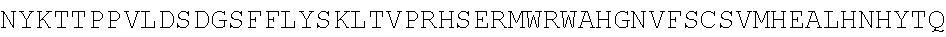

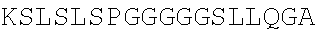

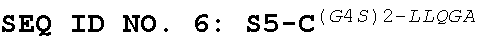

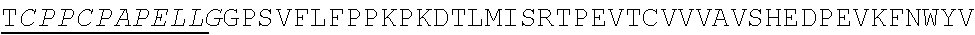

DGVEVHNAKTKPREEQYNSTYRVVSVLTVLHQDWLNGKEYKCKVSNKALPAPIEK

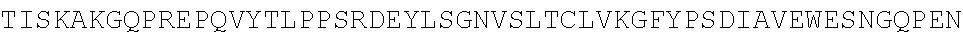

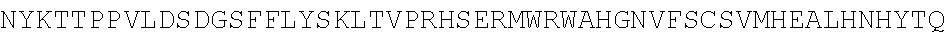

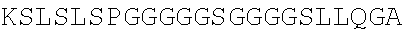

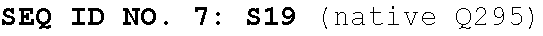

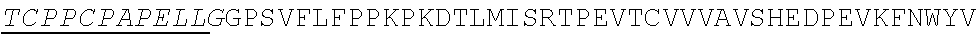

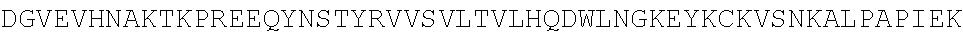

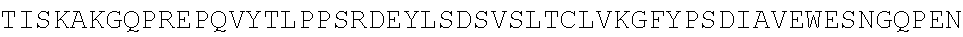

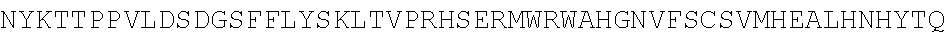

KSLSLSPG

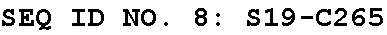

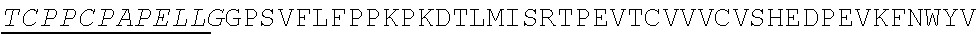

DGVEVHNAKTKPREEQYNSTYRVVSVLTVLHQDWLNGKEYKCKVSNKALPAPIEK

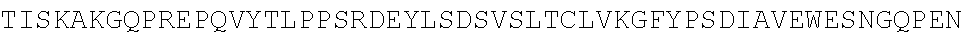

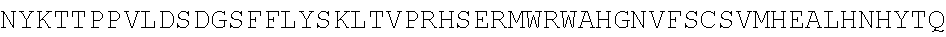

KSLSLSPG

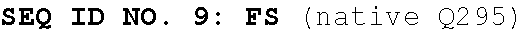

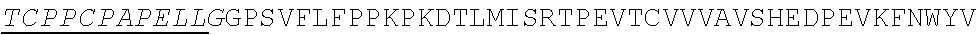

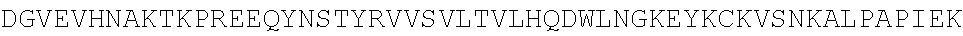

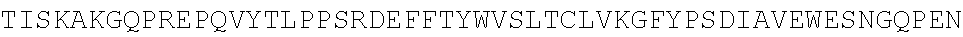

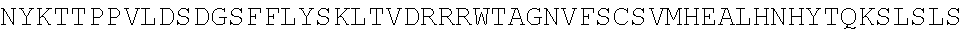

PG
Additional tested HER2 Fcab sequences
SEQ ID NO. 10: αH-H10 (Q295)
TCPPCPAPELLGGPSVFLFPPKPKDTLMISRTPEVTCVVVAVSHEDPEVKFNWYV
DGVEVHNAKTKPREEQYNSTYRVVSVLTVLHQDWLNGKEYKCKVSNKALPAPIEK

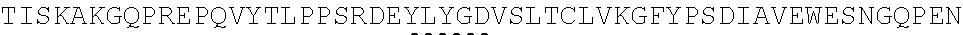

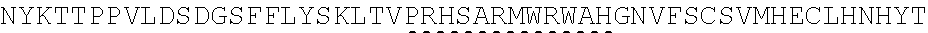

QKSLSLSGEC
SEQ ID NO. 11: αH-H10^{C265} (D265C)
TCPPCPAPELLGGPSVFLFPPKPKDTLMISRTPEVTCVVVCVSHEDPEVKFNWYV
DGVEVHNAKTKPREEQYNSTYRVVSVLTVLHQDWLNGKEYKCKVSNKALPAPIEK

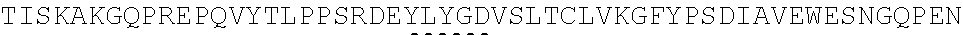

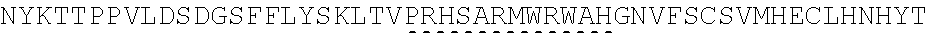

QKSLSLSGEC

### Additional Publicly Available HER2 Fcab Sequences

huFc Fragment with C_{H}3 AB Loop Light Grey and CH3 EF Loop Dark Grey

SEQ ID NO. 12: H242-9 (taken from 10.1093/protein/gzq005)
TCPPCPAPELLGGPSVFLFPPKPKDTLMISRTPEVTCVVVAVSHEDPEVKFNWYV
DGVEVHNAKTKPREEQYNSTYRVVSVLTVLHQDWLNGKEYKCKVSNKALPAPIEK

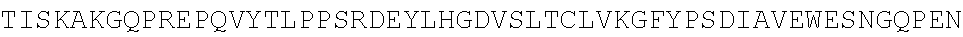

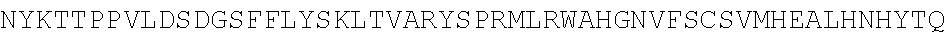

KSLSLSPG
SEQ ID NO. 13: STAB1 (taken from 10.1093/protein/gzs102)
TCPPCPAPELLGGPSVFLFPPKPKDTLMISRTPEVTCVVVAVSHEDPEVKFNWYV
DGVEVHNAKTKPREEQYNSTYRVVSVLTVLHQDWLNGKEYKCKVSNKALPAPIEK

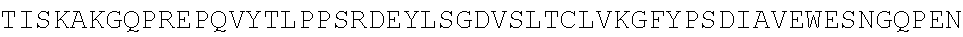

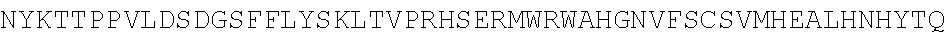

KSLSLSPG
SEQ ID NO. 14: STAB11 (taken from 10.1093/protein/gzs102)
TCPPCPAPELLGGPSVFLFPPKPKDTLMISRTPEVTCVVVAVSHEDPEVKFNWYV
DGVEVHNAKTKPREEQYNSTYRVVSVLTVLHQDWLNGKEYKCKVSNKALPAPIEK

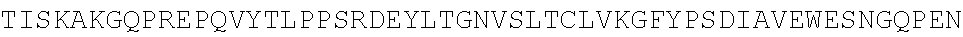

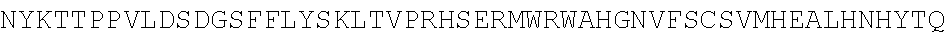

KSLSLSPG
SEQ ID NO. 15: STAB14 (taken from 10.1093/protein/gzs102)
TCPPCPAPELLGGPSVFLFPPKPKDTLMISRTPEVTCVVVAVSHEDPEVKFNWYV
DGVEVHNAKTKPREEQYNSTYRVVSVLTVLHQDWLNGKEYKCKVSNKALPAPIEK

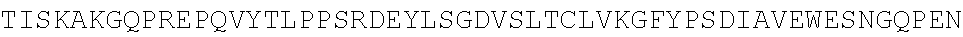

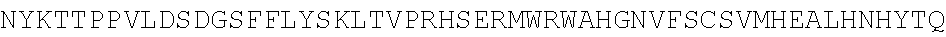

KSLSLSPG
SEQ ID NO. 16: STAB15 (taken from 10.1093/protein/gzs102)
TCPPCPAPELLGGPSVFLFPPKPKDTLMISRTPEVTCVVVAVSHEDPEVKFNWYV
DGVEVHNAKTKPREEQYNSTYRVVSVLTVLHQDWLNGKEYKCKVSNKALPAPIEK

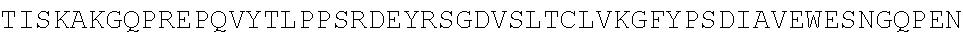

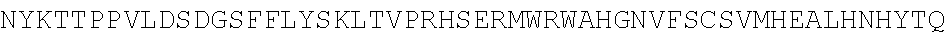

KSLSLSPG

### 2. Reference and Control Molecules

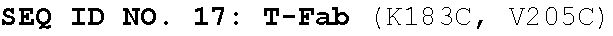

Light Chain:
DIQMTQSPSSLSASVGDRVTITCRASQDVNTAVAWYQQKPGKAPKLLIYSASFLYS
GVPSRFSGSRSGTDFTLTISSLQPEDFATYYCQQHYTTPPTFGQGTKVEIKRTVAA
PSVFIFPPSDEQLKSGTASVVCLLNNFYPREAKVQWKVDNALQSGNSQESVTEQD

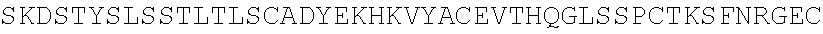

Heavy Chain:
EVOLVESGGGLVQPGGSLRLSCAASGFNIKDTYIHWVRQAPGKGLEWVARIYPTN
GYTRYADSVKGRFTISADTSKNTAYLQMNSLRAEDTAVYYCSRWGGDGFYAMDY
WGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKKVE
PKSCDKTHTCPPCPAPELLGHHHHHH

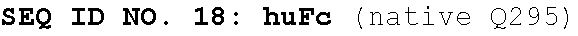

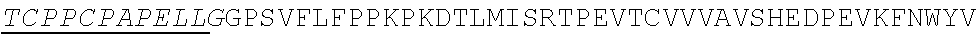

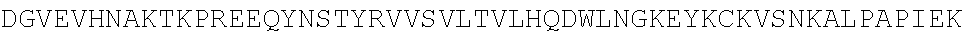

TISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSL
SPG

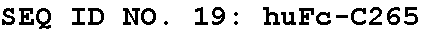

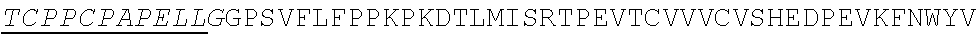

DGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSL
SPG

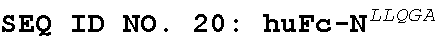

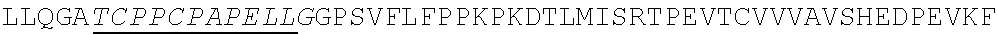

NWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQK
SLSLSPG

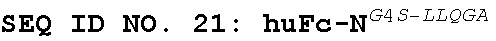

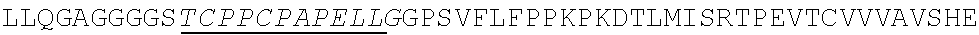

DPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALH
NHYTQKSLSLSPG
α-HEL-S5
SEQ ID NO. 22: Light Chain:
DIQMTQSPSSLSASVGDRVTITCRASGNIHNYLAWYQQKPGKAPKLLIYYTTTLAD
GVPSRFSGSGSGTDYTFTISSLQPEDIATYYCQHFWSTPRTFGQGTKVEIKRTVAA
PSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
SEQ ID NO. 23: Heavy Chain:
QVQLQESGPGLVRPSQTLSLTCTVSGFSLTGYGVNWVRQPPGRGLEWIGMIWGD
GNTDYNSALKSRVTMLKDTSKNQFSLRLSSVTAADTAVYYCARERDYRLDYWGQ
GSLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKS

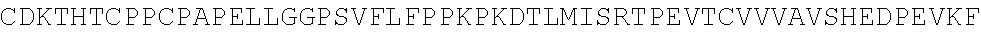

NWYVDGVEVHNAKTKPREEQYNSTYRVVSVLTVLHQDWLNGKEYKCKVSNKALP

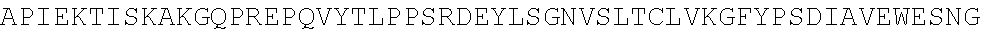

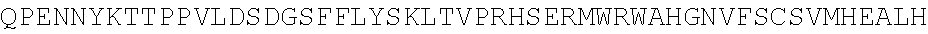

NHYTQKSLSLSPG

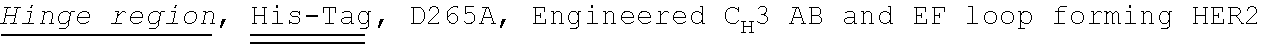

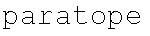

### REFERENCES

(1) Boni, V., Sharma, M. R., Patnaik, A. (2020) The Resurgence of Antibody Drug Conjugates in Cancer Therapeutics: Novel Targets and Payloads. Am. Soc. Clin. Oncol. Educ. B. No. 40, e58-e74.
(2) Syed, Y. Y. (2020) Sacituzumab Govitecan: First Approval. Drugs 80, 1019-1025.
(3) Mullard, A. (2020) FDA Approves First BCMA-Targeted Therapeutic. Nat. Rev. Drug Discov. 19, 659-659.
(4) Khongorzul, P., Ling, C. J., Khan, F. U., Ihsan, A. U., Zhang, J. (2020) Antibody-Drug Conjugates: A Comprehensive Review. Mol. Cancer Res. 18, 3-19.
(5) Deonarain, M. P. (2018) Miniaturised ‘Antibody’-Drug Conjugates for Solid Tumours? Drug Discov. Today Technol. 30, 47-53.
(6) Deonarain, M., Yahioglu, G., Stamati, I., Pomowski, A., Clarke, J., Edwards, B., Diez-Posada, S., Stewart, A. (2018) Small-Format Drug Conjugates: A Viable Alternative to ADCs for Solid Tumours? Antibodies 7, 16.
(7) Ruddle, B. T., Fleming, R., Wu, H., Gao, C., Dimasi, N. (2019) Characterization of Disulfide Bond Rebridged Fab-Drug Conjugates Prepared Using a Dual Maleimide Pyrrolobenzodiazepine Cytotoxic Payload. ChemMedChem 14, 1185-1195.
(8) Liu, W., Zhao, W., Bai, X., Jin, S., Li, Y., Qiu, C., Pan, L., Ding, D., Xu, Y., Zhou, Z., et al. (2019) High Antitumor Activity of Sortase A-Generated Anti-CD20 Antibody Fragment Drug Conjugates. Eur. J. Pharm. Sci. 134, 81-92.
(9) Higgins, J. P., Sarkar, A., Williams, E. T., Iberg, A., Waltzman, R., Willert, E. K. Abstract P1-18-35: MT-5111, a Novel HER2 Targeting Engineered Toxin Body, under Clinical Development to Overcome Mechanisms of Resistance to Existing HER2 Targeted Therapies. In Poster Session Abstracts; American Association for Cancer Research; pp P1-18-35.
(10) Aubrey, N., Allard-Vannier, E., Martin, C., Bryden, F., Letast, S., Colas, C., Lakhrif, Z., Collinet, N., Dimier-Poisson, I., Chourpa, I., et al. (2018) Site-Specific Conjugation of Auristatins onto Engineered ScFv Using Second Generation Maleimide to Target HER2-Positive Breast Cancer in Vitro. Bioconjug. Chem. 29, 3516-3521.
(11) Li, Q., Barrett, A., Vijayakrishnan, B., Tiberghien, A., Beard, R., Rickert, K. W., Allen, K. L., Christie, R. J., Marelli, M., Harper, J., et al. (2019) Improved Inhibition of Tumor Growth by Diabody-Drug Conjugates via Half-Life Extension. Bioconjug. Chem. 30, 1232-1243.
(12) Peretti, S. Abdurin-Drug Conjugates: A New Generation of Targeted Therapies. In Abdurin-Drug Conjugates—Small Size and Long Half-Life to Improve Drug Concentration in the Target Tissue; PEGS Europe: Lisbon.
(13) Glassman, P. M., Walsh, L. R., Villa, C. H., Marcos-Contreras, O. A., Hood, E. D., Muzykantov, V. R., Greineder, C. F. (2020) Molecularly Engineered Nanobodies for Tunable Pharmacokinetics and Drug Delivery. Bioconjug. Chem. 31, 1144-1155.
(14) Huang, H., Wu, T., Shi, H., Wu, Y., Yang, H., Zhong, K., Wang, Y., Liu, Y. (2019) Modular Design of Nanobody-Drug Conjugates for Targeted-Delivery of Platinum Anticancer Drugs with an MRI Contrast Agent. Chem. Commun. 55, 5175-5178.
(15) Cao, L., Li, Q., Tong, Z., Xing, Y., Xu, K., Yijia Wang, J., Li, W., Zhao, J., Zhao, L., Hong, Z. (2020) HER2-Specific Immunotoxins Constructed Based on Single-Domain Antibodies and the Improved Toxin PE24X7. Int. J. Pharm. 574, 118939.
(16) Nessler, I., Khera, E., Vance, S., Kopp, A., Qiu, Q., Keating, T. A., Abu-Yousif, A. O., Sandal, T., Legg, J., Thompson, L., et al. (2020) Increased Tumor Penetration of Single-Domain Antibody-Drug Conjugates Improves In Vivo Efficacy in Prostate Cancer Models. Cancer Res. 80, 1268-1278.
(17) Li, Z., Li, Y., Chang, H. P., Chang, H. Y., Guo, L., Shah, D. K. (2019) Effect of Size on Solid Tumor Disposition of Protein Therapeutics. Drug Metab. Dispos. 47, 1136-1145.
(18) Thurber, G. M., Schmidt, M. M., Wittrup, K. D. (2008) Antibody Tumor Penetration: Transport Opposed by Systemic and Antigen-Mediated Clearance. Adv. Drug Deliv. Rev. 60, 1421-1434.
(19) Brandl, F., Busslinger, S., Zangemeister-Wittke, U., Plückthun, A. (2020) Optimizing the Anti-Tumor Efficacy of Protein-Drug Conjugates by Engineering the Molecular Size and Half-Life. J. Control. Release 327, 186-197.
(20) Hirasawa, S., Kitahara, Y., Okamatsu, Y., Fujii, T., Nakayama, A., Ueno, S., Ijichi, C., Futaki, F., Nakata, K., Taki, M. (2019) Facile and Efficient Chemoenzymatic Semisynthesis of Fc-Fusion Compounds for Half-Life Extension of Pharmaceutical Components. Bioconjug. Chem. 30, 2323-2331.
(21) Lobner, E., Traxlmayr, M. W., Obinger, C., Hasenhindl, C. (2016) Engineered IgG1-Fc-One Fragment to Bind Them All. Immunol. Rev. 270, 113-131.
(22) Wozniak-Knopp, G., Bartl, S., Bauer, A., Mostageer, M., Woisetschläger, M., Antes, B., Ettl, K., Kainer, M., Weberhofer, G., Wiederkum, S., et al. (2010) Introducing Antigen-Binding Sites in Structural Loops of Immunoglobulin Constant Domains: Fc Fragments with Engineered HER2/Neu-Binding Sites and Antibody Properties. Protein Eng. Des. Sel. 23, 289-297.
(23) Lobner, E., Humm, A.-S., Göritzer, K., Mlynek, G., Puchinger, M. G., Hasenhindl, C., Rüker, F., Traxlmayr, M. W., Djinović-Carugo, K., Obinger, C. (2017) Fcab-HER2 Interaction: A Ménage à Trois. Lessons from X-Ray and Solution Studies. Structure 25, 878-889.
(24) Leung, K. M., Batey, S., Rowlands, R., Isaac, S. J., Jones, P., Drewett, V., Carvalho, J., Gaspar, M., Weller, S., Medcalf, M., et al. (2015) A HER2-Specific Modified Fc Fragment (Fcab) Induces Antitumor Effects through Degradation of HER2 and Apoptosis. Mol. Ther. 23, 1722-1733.
(25) Woisetschläger, M., Antes, B., Borrowdale, R., Wiederkum, S., Kainer, M., Steinkellner, H., Wozniak-Knopp, G., Moulder, K., Rüker, F., Mudde, G. C. (2014) In Vivo and in Vitro Activity of an Immunoglobulin Fc Fragment (Fcab) with Engineered Her-2/Neu Binding Sites. Biotechnol. J. 9, 844-851.
(26) Traxlmayr, M. W., Lobner, E., Hasenhindl, C., Stadlmayr, G., Oostenbrink, C., Rüker, F., Obinger, C. (2014) Construction of PH-Sensitive Her2-Binding IgG1-Fc by Directed Evolution. Biotechnol. J. 9, 1013-1022.
(27) Traxlmayr, M. W., Lobner, E., Antes, B., Kainer, M., Wiederkum, S., Hasenhindl, C., Stadlmayr, G., Rüker, F., Woisetschläger, M., Moulder, K., et al. (2013) Directed Evolution of Her2/Neu-Binding IgG1-Fc for Improved Stability and Resistance to Aggregation by Using Yeast Surface Display. Protein Eng. Des. Sel. 26, 255-265.
(28) Anderl, J., Torsten, H., Müller, C., Pahl, A. Amatoxin-Antibody Conjugates. US20180043033A1, 2018.
(29) Strop, P., Dorywalska, M., Rajpal, A., Shelton, D., Liu, S.-H., Pons, J., Dushin, R. Engineered Polypeptide Conjugates and Methods for Making Thereof Using Transglutaminase. WO2012059882A2, 2012.
(30) Baudino, L., Shinohara, Y., Nimmerjahn, F., Furukawa, J., Nakata, M., Martínez-Soria, E., Petry, F., Ravetch, J. V, Nishimura, S., Izui, S. (2008) Crucial Role of Aspartic Acid at Position 265 in the CH2 Domain for Murine IgG2a and IgG2b Fc-Associated Effector Functions. J. Immunol. 181, 6664-6669.
(31) Shields, R. L., Namenuk, A. K., Hong, K., Meng, Y. G., Rae, J., Briggs, J., Xie, D., Lai, J., Stadlen, A., Li, B., et al. (2001) High Resolution Mapping of the Binding Site on Human IgG1 for FcγRI, FcγRII, FcγRIII, and FcRn and Design of IgG1 Variants with Improved Binding to the FcγR. J. Biol. Chem. 276, 6591-6604.
(32) Mahalingaiah, P. K., Ciurlionis, R., Durbin, K. R., Yeager, R. L., Philip, B. K., Bawa, B., Mantena, S. R., Enright, B. P., Liguori, M. J., Van Vleet, T. R. (2019) Potential Mechanisms of Target-Independent Uptake and Toxicity of Antibody-Drug Conjugates. Pharmacol. Ther. 200, 110-125.
(33) Marquette, K., Bennett, E., Tchistiakova, L., Tumey, L. N., Bikker, J., Calabro, V., Graziani, E. Engineered Antibody Constant Regions for Site-Specific Conjugation and Methods and Uses Therefor. EP2794653B1, 2019.
(34) Vollmar, B. S., Wei, B., Ohri, R., Zhou, J., He, J., Yu, S.-F., Leipold, D., Cosino, E., Yee, S., Fourie-O'Donohue, A., et al. (2017) Attachment Site Cysteine Thiol PKa Is a Key Driver for Site-Dependent Stability of THIOMAB Antibody-Drug Conjugates. Bioconjug. Chem. 28, 2538-2548.
(35) Nath, N., Godat, B., Zimprich, C., Dwight, S. J., Corona, C., McDougall, M., Urh, M. (2016) Homogeneous Plate Based Antibody Internalization Assay Using PH Sensor Fluorescent Dye. J. Immunol. Methods 431, 11-21.
(36) Owen, S. C., Patel, N., Logie, J., Pan, G., Persson, H., Moffat, J., Sidhu, S. S., Shoichet, M. S. (2013) Targeting HER2+Breast Cancer Cells: Lysosomal Accumulation of Anti-HER2 Antibodies Is Influenced by Antibody Binding Site and Conjugation to Polymeric Nanoparticles. J. Control. Release 172, 395-404.
(37) Kang, J. C., Sun, W., Khare, P., Karimi, M., Wang, X., Shen, Y., Ober, R. J., Ward, E. S. (2019) Engineering a HER2-Specific Antibody-Drug Conjugate to Increase Lysosomal Delivery and Therapeutic Efficacy. Nat. Biotechnol. 37, 523-526.
(38) Ram, S., Kim, D., Ober, R. J., Ward, E. S. (2014) The Level of HER2 Expression Is a Predictor of Antibody-HER2 Trafficking Behavior in Cancer Cells. MAbs 6, 1211-1219.
(39) Shen, B., Xu, K., Liu, L., Raab, H., Bhakta, S., Kenrick, M., Parsons-reponte, K. L., Tien, J., Yu, S., Mai, E., et al. (2012) Conjugation Site Modulates the in Vivo Stability and Therapeutic Activity of Antibody-Drug Conjugates. Nat. Biotechnol. 30, 184-189.
(40) Strop, P., Liu, S., Dorywalska, M., Delaria, K., Dushin, R. G., Tran, T., Ho, W., Farias, S., Casas, M. G., Abdiche, Y., et al. (2013) Location Matters: Site of Conjugation Modulates Stability and Pharmacokinetics of Antibody Drug Conjugates. Chem. Biol. 20, 161-167.
(41) Dorywalska, M., Strop, P., Melton-Witt, J. A., Hasa-Moreno, A., Farias, S. E., Galindo Casas, M., Delaria, K., Lui, V., Poulsen, K., Loo, C., et al. (2015) Effect of Attachment Site on Stability of Cleavable Antibody Drug Conjugates. Bioconjug. Chem. 26, 650-659.
(42) Dickgiesser, S., Rieker, M., Mueller-Pompalla, D., Schröter, C., Tonillo, J., Warszawski, S., Raab-Westphal, S., Kühn, S., Knehans, T., Könning, D., et al. (2020) Site-Specific Conjugation of Native Antibodies Using Engineered Microbial Transglutaminases. Bioconjug. Chem. 31, 1070-1076.
(43) Jeger, S., Zimmermann, K., Blanc, A., Grünberg, J., Honer, M., Hunziker, P., Struthers, H., Schibli, R. (2010) Site-Specific and Stoichiometric Modification of Antibodies by Bacterial Transglutaminase. Angew. Chemie Int. Ed. 49, 9995-9997.
(44) Tumey, L. N., Li, F., Rago, B., Han, X., Loganzo, F., Musto, S., Graziani, E. I., Puthenveetil, S., Casavant, J., Marquette, K., et al. (2017) Site Selection: A Case Study in the Identification of Optimal Cysteine Engineered Antibody Drug Conjugates. AAPS J. 19, 1123-1135.
(45) Tobias, R., Ma, W. Application Note 19: Analysis of FcRn-Antibody Interactions on the Octet Platform. PALL, FortéBio.
(46) Benjamin, S. R., Jackson, C. P., Fang, S., Carlson, D. P., Guo, Z., Tumey, L. N. (2019) Thiolation of Q295: Site-Specific Conjugation of Hydrophobic Payloads without the Need for Genetic Engineering. Mol. Pharm. 16, 2795-2807.
(47) Dorywalska, M., Dushin, R., Moine, L., Farias, S. E., Zhou, D., Navaratnam, T., Lui, V., Hasa-Moreno, A., Casas, M. G., Tran, T.-T., et al. (2016) Molecular Basis of Valine-Citrulline-PABC Linker Instability in Site-Specific ADCs and Its Mitigation by Linker Design. Mol. Cancer Ther. 15, 958-970.
(48) Anami, Y., Yamazaki, C. M., Xiong, W., Gui, X., Zhang, N., An, Z., Tsuchikama, K. (2018) Glutamic Acid-Valine-Citrulline Linkers Ensure Stability and Efficacy of Antibody-Drug Conjugates in Mice. Nat. Commun. 9, 2512.
(49) Thurber, G. M., Schmidt, M. M., Wittrup, K. D. (2008) Factors Determining Antibody Distribution in Tumors. Trends Pharmacol. Sci. 29, 57-61.
(50) Trastuzumab amino acid sequence on DrugBank database (accession number: DB00072) https://www.drugbank.ca/drugs/DB00072 (accessed Jun. 14, 2020).
(51) Baggethun, P. Radial Profile Plot for ImageJ https://imagej.nih.gov/ij/plugins/radial-profile.html (accessed Apr. 28, 2020).
(52) Traxlmayr, M. W., Lobner, E., Antes, B., Kainer, M., Wiederkum, S., Hasenhindl, C., Stadlmayr, G., Rüker, F., Woisetschläger, M., Moulder, K., et al. (2013) Directed Evolution of Her2/Neu-Binding IgG1-Fc for Improved Stability and Resistance to Aggregation by Using Yeast Surface Display. Protein Eng. Des. Sel. 26, 255-265.
(53) Leung, K. M., Batey, S., Rowlands, R., Isaac, S. J., Jones, P., Drewett, V., Carvalho, J., Gaspar, M., Weller, S., Medcalf, M., et al. (2015) A HER2-Specific Modified Fc Fragment (Fcab) Induces Antitumor Effects through Degradation of HER2 and Apoptosis. Mol. Ther. 23, 1722-1733.
(54) Lobner, E., Humm, A.-S., Göritzer, K., Mlynek, G., Puchinger, M. G., Hasenhindl, C., Rüker, F., Traxlmayr, M. W., Djinović-Carugo, K., Obinger, C. (2017) Fcab-HER2 Interaction: A Ménage à Trois. Lessons from X-Ray and Solution Studies. Structure 25, 878-889.
(55) Trastuzumab amino acid sequence on DrugBank database (accession number: DB00072) https://www.drugbank.ca/drugs/DB00072 (accessed Jun. 14, 2020).
(56) Baggethun, P. Radial Profile Plot for ImageJ https://imagej.nih.gov/ij/plugins/radial-profile.html (accessed Apr. 28, 2020).

1. HER2 Fcab-drug conjugate or a pharmaceutically acceptable salt thereof, comprising the formula Fcab-(L)ₘ-(D)ₙ wherein:
a) Fcab comprises a HER2 Fcab,
b) L comprises a linker,
c) D comprises a drug,
d) m is an integer from 1-5 and n is an integer from 1-10.

2. HER2 Fcab-drug conjugate according to claim 1 wherein the HER2 Fcab is selected from the group consisting of: S5 (native Q295), S5-C265, S5-N^{LLQGA}, S5-N^{G4S-LLQGA}, S5-C^{G4S-LLQGA}, S5-C^{(G4S)2-LLQGA}, S19 (native Q295), S19 (native Q295), FS (native Q295), αH-H10 (Q295), αH-H10^{C265} (D265C), H242-9, STAB1, STAB11, STAB14 and STAB15, having the amino acid sequences as set forth in SEQ ID Nos. 1-16.

3. HER2 Fcab-label conjugate comprising the formula Fcab-(L)ₘ-(La)ₙ wherein:
a) Fcab comprises a HER2 Fcab,
b) L comprises a linker,
c) La comprises a label,
d) m is an integer from 1-5 and n is an integer from 1-10.

4. Pharmaceutical composition comprising at least one HER2 Fcab-drug conjugate according to claim 1.

5. Pharmaceutical composition according to claim 4 comprising further excipients and/or adjuvants.

6. Pharmaceutical composition comprising at least one HER2 Fcab-drug conjugate according to claim 1 and at least one further pharmaceutically active compound.

7. Process for the preparation of a pharmaceutical preparation, characterised in that a HER2 Fcab-drug conjugate according to claim 1 is brought into a suitable dosage form together with a solid, liquid or semi-liquid excipient or adjuvant.

8. Diagnostic composition comprising at least one HER2 Fcab-label conjugate according to claim 3.

9. A method for the treatment and/or prophylaxis of physiological and/or pathophysiological states, comprising administering to a subject in need thereof an effective amount of at least one HER2 Fcab-drug conjugate according to claim 1.

10. A method for the treatment and/or prophylaxis of hyperproliferative diseases and/or disorders, comprising administering to a subject in need thereof an effective amount of at least one HER2 Fcab-drug conjugate according to claim 1.

11. A method according to claim 10, wherein the hyperproliferative disease or disorder is cancer.

12. A method according to claim 11, wherein the cancer is a HER2-positive cancer.

13. A method according to claim 11, wherein the cancer is selected from the group consisting of acute and chronic lymphocytic leukemia, acute granulocytic leukemia, adrenal cortex cancer, bladder cancer, brain cancer, breast cancer, cervical cancer, cervical hyperplasia, chorion cancer, chronic granulocytic leukemia, chronic lymphocytic leukemia, colon cancer, endometrial cancer, kidney cancer, biliary tract cancer, hepatoma, liver cancer, esophageal cancer, essential thrombocytosis, genitourinary carcinoma, glioma, glioblastoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, chondrosarcoma, multiple myeloma, mycosis fungoides, myeloid and lymphocytic leukemia, neuroblastoma, non-Hodgkin's lymphoma, non-small cell lung cancer, osteogenic sarcoma, ovarian carcinoma, pancreatic carcinoma, polycythemia vera, primary brain carcinoma, primary macroglobulinemia, prostatic cancer, renal cell cancer, rhabdomyosarcoma, skin cancer, small-cell lung cancer, soft-tissue sarcoma, squamous cell cancer, stomach cancer, testicular cancer, thyroid cancer and Wilms' tumor.

14. A method according to claim 11, wherein the cancer is selected from the group consisting of breast cancer, gastric cancer, stomach cancer, colorectal cancer, ovarian cancer, pancreatic cancer, endometrial cancer or non-small cell lung cancer.

15. Set (kit) consisting of separate packs of
a) at least one HER2 Fcab-drug conjugate according to claim 1, and
b) aa further pharmaceutically active compound.


SEQUENCE LISTING (23 sequences)

SEQ ID NO: 1 - protein, 227 aa, Artificial Sequence
  S5 (native Q295)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSGNVSLTCLVKGFYPSDIAVEWESNGQPENNYKTTPPVLDSDGSFFLYSKLTVPRHSERMWRWAHGNVFSCSVMHEALHNHYTQKSLSLSPG

SEQ ID NO: 2 - protein, 227 aa, Artificial Sequence
  S5-C265
TCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSGNVSLTCLVKGFYPSDIAVEWESNGQPENNYKTTPPVLDSDGSFFLYSKLTVPRHSERMWRWAHGNVFSCSVMHEALHNHYTQKSLSLSPG

SEQ ID NO: 3 - protein, 232 aa, Artificial Sequence
  S5-NLLQGA
LLQGATCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSGNVSLTCLVKGFYPSDIAVEWESNGQPENNYKTTPPVLDSDGSFFLYSKLTVPRHSERMWRWAHGNVFSCSVMHEALHNHYTQKSLSLSPG

SEQ ID NO: 4 - protein, 237 aa, Artificial Sequence
  S5-NG4S-LLQGA
LLQGAGGGGSTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSGNVSLTCLVKGFYPSDIAVEWESNGQPENNYKTTPPVLDSDGSFFLYSKLTVPRHSERMWRWAHGNVFSCSVMHEALHNHYTQKSLSLSPG

SEQ ID NO: 5 - protein, 237 aa, Artificial Sequence
  S5-CG4S-LLQGA
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSGNVSLTCLVKGFYPSDIAVEWESNGQPENNYKTTPPVLDSDGSFFLYSKLTVPRHSERMWRWAHGNVFSCSVMHEALHNHYTQKSLSLSPGGGGGSLLQGA

SEQ ID NO: 6 - protein, 242 aa, Artificial Sequence
  S5-C(G4S)2-LLQGA
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSGNVSLTCLVKGFYPSDIAVEWESNGQPENNYKTTPPVLDSDGSFFLYSKLTVPRHSERMWRWAHGNVFSCSVMHEALHNHYTQKSLSLSPGGGGGSGGGGSLLQGA

SEQ ID NO: 7 - protein, 227 aa, Artificial Sequence
  S19 (native Q295)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSDSVSLTCLVKGFYPSDIAVEWESNGQPENNYKTTPPVLDSDGSFFLYSKLTVPRHSETMRRWAHGNVFSCSVMHEALHNHYTQKSLSLSPG

SEQ ID NO: 8 - protein, 227 aa, Artificial Sequence
  S19-C265
TCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSDSVSLTCLVKGFYPSDIAVEWESNGQPENNYKTTPPVLDSDGSFFLYSKLTVPRHSETMRRWAHGNVFSCSVMHEALHNHYTQKSLSLSPG

SEQ ID NO: 9 - protein, 222 aa, Artificial Sequence
  FS (native Q295)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEFFTYWVSLTCLVKGFYPSDIAVEWESNGQPENNYKTTPPVLDSDGSFFLYSKLTVDRRRWTAGNVFSCSVMHEALHNHYTQKSLSLSPG

SEQ ID NO: 10 - protein, 228 aa, Artificial Sequence
  alphaH H10 (Q295)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQCREPQVYTLPPSRDEYLYGDVSLTCLVKGFYPSDIAVEWESNGQPENNYKTTPPVLDSDGSFFLYSKLTVPRHSARMWRWAHGNVFSCSVMHECLHNHYTQKSLSLSGEC

SEQ ID NO: 11 - protein, 228 aa, Artificial Sequence
  alphaH H10C265 (D265C)
TCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQCREPQVYTLPPSRDEYLYGDVSLTCLVKGFYPSDIAVEWESNGQPENNYKTTPPVLDSDGSFFLYSKLTVPRHSARMWRWAHGNVFSCSVMHECLHNHYTQKSLSLSGEC

SEQ ID NO: 12 - protein, 227 aa, Artificial Sequence
  H242-9 (taken from 10.1093/protein/gzq005)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLHGDVSLTCLVKGFYPSDIAVEWESNGQPENNYKTTPPVLDSDGSFFLYSKLTVARYSPRMLRWAHGNVFSCSVMHEALHNHYTQKSLSLSPG

SEQ ID NO: 13 - protein, 227 aa, Artificial Sequence
  STAB1 (taken from 10.1093/protein/gzs102)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSGDVSLTCLVKGFYPSDIAVEWESNGQPENNYKTTPPVLDSDGSFFLYSKLTVPRHSERMWRWAHGNVFSCSVMHEALHNHYTQKSLSLSPG

SEQ ID NO: 14 - protein, 227 aa, Artificial Sequence
  STAB11 (taken from 10.1093/protein/gzs102)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLTGNVSLTCLVKGFYPSDIAVEWESNGQPENNYKTTPPVLDSDGSFFLYSKLTVPRHSERMWRWAHGNVFSCSVMHEALHNHYTQKSLSLSPG

SEQ ID NO: 15 - protein, 227 aa, Artificial Sequence
  STAB14 (taken from 10.1093/protein/gzs102)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSGDVSLTCLVKGFYPSDIAVEWESNGQPENNYKTTPPVLDSDGSFFLYSKLTVPRHSDAMRRWAHGNVFSCSVMHEALHNHYTQKSLSLSPG

SEQ ID NO: 16 - protein, 227 aa, Artificial Sequence
  STAB15 (taken from 10.1093/protein/gzs102)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYRSGDVSLTCLVKGFYPSDIAVEWESNGQPENNYKTTPPVLDSDGSFFLYSKLTVPRHSERMWRWAHGNVFSCSVMHEALHNHYTQKSLSLSPG

SEQ ID NO: 17 - protein, 214 aa, Artificial Sequence
  T-Fab (K183C, V205C)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSCADYEKHKVYACEVTHQGLSSPCTKSFNRGEC

SEQ ID NO: 18 - protein, 222 aa, Artificial Sequence
  huFc (native Q295)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 222 aa, Artificial Sequence
  huFc-C265
TCPPCPAPELLGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 227 aa, Artificial Sequence
  huFc-NLLQGA
LLQGATCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 232 aa, Artificial Sequence
  huFc-NG4S-LLQGA
LLQGAGGGGSTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 214 aa, Artificial Sequence
  Light Chain
DIQMTQSPSSLSASVGDRVTITCRASGNIHNYLAWYQQKPGKAPKLLIYYTTTLADGVPSRFSGSGSGTDYTFTISSLQPEDIATYYCQHFWSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 450 aa, Artificial Sequence
  Heavy Chain
QVQLQESGPGLVRPSQTLSLTCTVSGFSLTGYGVNWVRQPPGRGLEWIGMIWGDGNTDYNSALKSRVTMLKDTSKNQFSLRLSSVTAADTAVYYCARERDYRLDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDEYLSGNVSLTCLVKGFYPSDIAVEWESNGQPENNYKTTPPVLDSDGSFFLYSKLTVPRHSERMWRWAHGNVFSCSVMHEALHNHYTQKSLSLSPG